(19) United States
(12) Patent Application Publication
(10) Pub. No.: US 2011/0071124 A1
(43) Pub. Date: Mar. 24, 2011

(54) Compounds that Inhibit Production of sAPPB and AB and Uses Thereof

(75) Inventors: Tae-wan Kim, East Brunswick, NJ (US); Donald W. Landry, New York, NY (US); Jeremy C. Hwang, Great Neck, NY (US); Shi Xian Deng, White Plains, NY (US); Gangli Gong, Little Neck, NY (US); Yuli Xie, New York, NY (US); Yidong Liu, New York, NY (US); Alison Rinderspacher, New York, NY (US)

(73) Assignee: The Trustees of Columbia University in the City of New York, New York (US)

(21) Appl. No.: 12/858,837

(22) Filed: Aug. 18, 2010

Related U.S. Application Data
(63) Continuation of application No. PCT/US2009/043009, filed on May 6, 2009.
(60) Provisional application No. 61/050,982, filed on May 6, 2008.
(60) Provisional application No. 61/143,400, filed on Jan. 8, 2009.

(51) Int. Cl. A61K 31/40; A61K 31/445; A61K 31/397; A61K 31/165; A61K 31/138; A61K 31/495; A61K 31/41; A61K 31/4184; A61K 31/35; A61P 25/28; C12N 9/99

(52) U.S. Cl. 514/210.01; 514/428; 514/423; 514/331; 514/330; 514/210.17; 514/617; 514/648; 514/255.04; 514/381; 514/395; 514/460; 435/184

(57) ABSTRACT

The present invention relates to compounds with activity as inhibitors of sAPPβ and Aβ production, and methods for treating, preventing, or ameliorating neurodegenerative diseases, such as Alzheimer's disease and pharmaceutical compositions containing such candidate compounds.

### CROSS-REFERENCE TO RELATED APPLICATIONS

The present application claims the benefit of U.S. Provisional Application No. 61/050,982, filed May 6, 2008; and U.S. Provisional Application No. 61/143,400, filed Jan. 8, 2009, each of which is hereby incorporated by reference in their entireties.

### GRANT INFORMATION

This invention was made with government support under grants 5 U24 NS049339-03, P50 AG08702, and 5RO1 AT001643 awarded by the National Institutes of Health and the Molecular Libraries Initiative of the National Institutes of Health Roadmap for Medical Research. The government has certain rights in the invention.

### 1. INTRODUCTION

The present invention relates to compounds with activity as sAPPβ and Aβ production inhibitors. The present invention also relates to methods for treating, preventing, and/or ameliorating neurodegenerative diseases, such as Alzheimer's disease, using such compounds.

### 2. BACKGROUND OF THE INVENTION

### 2.1 Neurodegeneration and Alzheimer's Disease

Alzheimer's Disease is a progressive neurodegenerative disease characterized by progressive memory deficits, impaired cognitive function, altered and inappropriate behavior, and a progressive decline in language function. It is the most prevalent age-related dementia, affecting an estimated 18 million people worldwide, according to the World Health Organization. As medical advances continue to prolong the human lifespan, it is certain that AD will affect an increasing proportion of the population. Current FDA-approved therapies provide only temporary and symptomatic relief, while doing little to counteract disease progression.

Neuropathology findings in AD patients include cortical atrophy, loss of neurons and synapses, and hallmark extracellular senile plaques and intracellular neurofibrillary tangles. Senile (or neuritic) plaques are composed of aggregated amyloid β-peptide (Aβ), and are found in large numbers in the limbic and association cortices (Selkoe, 2001, Physiol Rev. 81:741-766). It is widely hypothesized that the extracellular accumulation of Aβ contributes to axonal and dendritic injury and subsequent neuronal death. Neurofibrillary tangles consist of pairs of filaments, which are about 10 nm in length, wound into helices (paired helical filaments or PHF). Immunohistochemical and biochemical analysis of neurofibrillary tangles revealed that they are composed of a hyperphosphorylated form of the microtubule-associated protein tau. These two classical pathological lesions of AD can occur independently of each other (Selkoe, 2001, Physiol Rev. 81:741-766). However, there is growing evidence that the gradual accumulation of Aβ and Aβ-associated molecules leads to the formation of neurofibrillary tangles. As such, much research is directed at inhibiting the generation of the amyloid β-peptide.

Aβ is derived from the sequential cleavage of amyloid precursor protein (APP) by membrane-bound proteases known as β-secretase and γ-secretase. A competing proteolytic pathway to the β-secretase pathway exists, the α-secretase pathway, which results in cleavage of APP within the Aβ domain, thereby precluding the generation of Aβ. β-site APP cleavage enzyme 1 (BACE1) was identified as the major β-secretase activity that mediates the first cleavage of APP in the β-amyloidgenic pathway (Hussain et al., 1999, MoI Cell Neurosci. 14:419-427; Sinha et al., 1999, Nature. 402:537-540; Vassar et al., 1999, Science 286:735-741; Yan, et al., 1999, Nature. 402:533-537).

BACE1 is a 501 amino acid protein that bears homology to eukaryotic aspartic proteases, especially from the pepsin family (Vassar, 2002, Advanced drug delivery reviews. 54:1589-1602). In common with other aspartic proteases, BACE1 is synthesized as a zymogen with a pro-domain that is cleaved by furin to release the mature protein. BACE1 is a type I transmembrane protein with a lumenal active site that cleaves APP to release an ectodomain (sAPPβ) into the extracellular space. The remaining C-terminal fragment (CTF) undergoes subsequent cleavage by γ-secretase to release Aβ and the APP intracellular C-terminal domain (AICD). The presenilins have been proposed to be the major enzymatic component of γ-secretase, whose imprecise cleavage of APP produces a spectrum of Aβ peptides varying in length by a few amino acids at the C-terminus. The majority of Aβ normally ends at amino acid 40 (Aβ40), but the 42-amino acid variant (Aβ42) has been shown to be more susceptible to aggregation, and has been hypothesized to nucleate senile plaque formation.

In light of the foregoing, BACE1 has become a popular research topic, and has, perhaps, surpassed γ-secretase as the most promising target for pharmaceutical research. Small molecule BACE1 inhibitors are being developed by numerous investigators. In particular, Hussain et. al. have demonstrated the in vivo efficacy of their BACE1 small molecule inhibitor, GSK188909, in a mouse model of AD (Hussain et al., 2007, J Neurochem. 100(3):802-9). While these results are promising, many challenges still remain. Because BACE1 has a large active site, it is difficult to design a compound large enough to achieve the high specificity required for a typical therapeutic, yet still small enough to effectively traverse the blood-brain barrier. In fact, because of low brain penetration, a p-glycoprotein inhibitor was required to facilitate transport of GSK188909 across the blood-brain barrier (Hussain et al., 2007, J Neurochem. 100(3):802-9). Accordingly, it remains desirable to identify a diverse set of small molecules which can reduce the cleavage of APP by BACE1 and thus the inhibit the production of Aβ.

### 3. SUMMARY OF THE INVENTION

The present invention relates to compounds which inhibit sAPPβ and Aβ activity. The compounds of the invention may be used to inhibit sAPPβ and Aβ activity in a subject, or in a cell in culture.

The present invention also provides a method for the treatment of a neurodegenerative condition, such as, but not limited to, Alzheimer's Disease in an individual, wherein the neurodegenerative condition is associated with β-amyloidogenic (Aβ) processing of Amyloid Precursor Protein (APP), by administering to an individual in need of such treatment a pharmaceutical composition comprising at least one compound of Formulas I-VII (meaning Formula I, II, III, IV, V, VI or VII), and/or at least one compound depicted in FIG. 19, in an amount effective to treat the neurodegenerative condition.

In a specific non-limiting embodiment, the individual has been diagnosed or is at risk of developing Alzheimer's disease (AD), including Familial or Sporadic faints of AD.

In still further non-limiting embodiments, the present invention relates to a compound of Formula I:

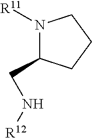

and to salts, esters and prodrugs of the compounds of Formula I. Additionally, the present invention describes methods of synthesizing and using compounds of Formula I.

In other non-limiting embodiments, the present invention relates to a compound of Formula II:

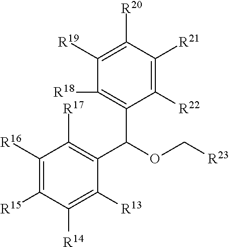

and salts, esters and prodrugs of the compounds of Formula II. Additionally, the present invention describes methods of synthesizing and using compounds of Formula II.

In other non-limiting embodiments, the present invention relates to a compound of Formula III:

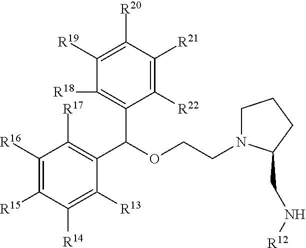

and salts, esters and prodrugs of the compounds of Formula III. Additionally, the present invention describes methods of synthesizing and using compounds of Formula III.

In other non-limiting embodiments, the present invention relates to a compound of Formula IV:

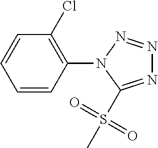

and salts, esters and prodrugs of the compounds of Formula IV. Additionally, the present invention describes methods of synthesizing and using compounds of Formula IV.

In other non-limiting embodiments, the present invention relates to a compound of the Formula V:

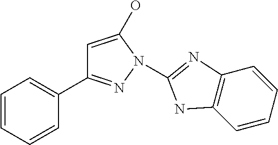

and salts, esters and prodrugs of the compounds of Formula V. Additionally, the present invention describes methods of synthesizing and using compounds of Formula V.

In other non-limiting embodiments, the present invention relates to a compound of Formula VI:

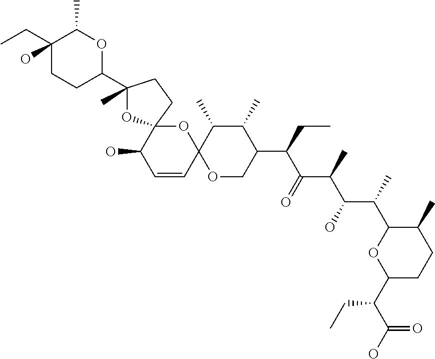

and salts, esters and prodrugs of the compounds of Formula VI. Additionally, the present invention describes methods of synthesizing and using compounds of Formula VI.

In other non-limiting embodiments, the present invention relates to a compound of Formula VII:

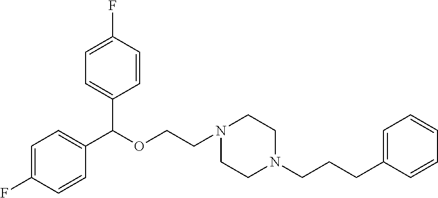

and salts, esters and prodrugs of the compounds of Formula VII. Additionally, the present invention describes methods of synthesizing and using compounds of Formula VII.

In other non-limiting embodiments, the present invention relates to one or more compounds depicted in FIG. 19, salts esters and prodrugs thereof, and methods of using these compounds.

The present invention further provides a method of inhibiting the activity of BACE1, by contacting the BACE1, or by contacting a cell expressing BACE1, with at least one compound of Formulas I-VII, and/or at least one compound depicted in FIG. 19, in an amount effective to inhibit the activity of BACE1.

In one non-limiting embodiment, the BACE1 is expressed by a cell, for example, a mammalian cell, e.g., a cell of a mammalian nervous system, and the cell is contacted with at least one compound of Formulas I-VII, and/or at least one compound depicted in FIG. 19.

The present invention also provides a method of decreasing β-site APP cleavage, and increasing the cleavage of APP by α-secretase, by contacting BACE1, or a cell expressing BACE1, with at least one compound of Formulas I-VII, and/or at least one compound depicted in FIG. 19, in an amount effective to increase the level of APP metabolism by α-secretase.

In another non-limiting embodiment, the compounds of the invention may be comprised in a pharmaceutical composition, and may optionally be used in conjunction with one or more additional compound for the treatment of a neurodegenerative condition, such as, but not limited to, Alzheimer's Disease.

### 4. BRIEF DESCRIPTION OF THE DRAWINGS

FIG. 1. An sAPPβ assay utilized to identify inhibitors of BACE1. The assay utilizes SY5Y cells stably overexpressing BACE-GFP and SEAP-APPwt. Cells are incubated with a candidate compound for 6 hours prior to harvesting media. BACE1-mediated cleavage of APP results in the release of sAPPβ into the media. α-secretase, a competing enzyme that is non-amyloidogenic, also cleaves APP to release sAPPβ. Using an sAPPβ-specific antibody (sβwt), SEAP-sAPPβ is specifically captured in a modified ELISA assay. Detection is achieved by addition of the fluorescent alkaline phosphatase substrate, 4-methylumbelliferyl phosphate (4-MUP).

FIG. 2. General structure of tagged-triazine compounds. The tri-substituted triazine is substituted at positions R3, R4 and R5, which may be the same or different. R3, R4, and R5 can be substituted or unsubstituted aryl, alkyl, alkenyl, alkynyl, or cyclic or heterocyclic group. With close structural similarity to purine and pyrimidine as well as three-fold symmetry, the triazine scaffold is an attractive starting point for combinatorial synthesis. The tagged-triazine compound library of Young-Tae Chang contains a built-in linker moiety for convenient attachment of an affinity tag after a hit is identified (WO 2004/099106).

FIG. 3A-C. Tagged-triazine library primary screen. The BACE1 assay was performed in 96-well format as described and used to conduct a medium-throughput screen of the 3000-compound tagged-triazine library. The assay was conducted without the assistance of robotic automation. A) Compounds were screened at 10 μM concentration (1% DMSO). Percent inhibition of sAPPβ was calculated by first subtracting the background signal from all data points, then according to the formula 100×[DMSO control−compound]/[DMSO control]. B) Z′ factor was calculated as described, based on the negative (DMSO) and positive (BACE inhibitor IV) control wells included in triplicate on every screening plate. Z′ factors from all plates exceeded the threshold of 0.5 for an excellent assay. Mean±s.d. values of Z′ were 0.83±0.08 (range=0.61-0.96), with a median Z′ factor of 0.86. C) Statistics from the primary screen. Due to the relatively small size of the library, the threshold value for hit selection was set at 2 s.d. above and below the mean, or 14.13% and -37.86%, to generate more hits. The resulting hit rate was 4.84%.

FIG. 4. Small molecule modulators of BACE1 activity. 144 hits were identified in the primary screen and retested at 10 μM in triplicate to confirm the activity. Because of the low threshold used for hit selection, only 3 compounds reconfirmed. TF-A6 and TG-CD-D7 caused a modest reduction of sAPPβ, while AM-D-E4 caused a modest increase in sAPPβ.

FIG. 5A-C. Primary screen of the LDDN small molecule library. The miniaturized cell-based sAPPβ ELISA assay was used to screen the LDDN small library, which consisted of roughly 400 384-well plates each containing 352 compounds, or a total of roughly 140,000 compounds. A) A representative 10,000 data points, showing that the majority of compounds were clustered between −30% and 30% sAPPβ inhibition. B) Z′ factors for all plates. The majority of Z′ factors exceeded the threshold of 0.5 for an excellent assay. Mean±s.d. of Z′ values were 0.67±0.11 (range=0.43-0.90), with a median value of 0.67. C) Statistics from the primary screen. The threshold value for hit selection was set at 4 standard deviations, or 52.94%, resulting in a hit rate of 0.11%.

FIG. 6. Confirmatory screen of LDDN hit compounds. Of the 147 hits selected during the primary screen, 139 were retested in a 3-point dose-response (10, 2, 0.2 μM) confirmatory screen. BACE1 assay was performed as described, and cell viability was determined by Cell Titer AQeous One cell proliferation assay (Promega). Representative data from four compounds are shown. Data points represent mean±s.d. of four determinations.

FIG. 7A-O. 15 LDDN hits.

FIG. 8. IC50 determination for LDDN hits. Hit compounds from the LDDN primary screen were characterized in 12-point dose-response experiments in the cell-based BACE1 assay for IC50 determination. Dose-response curves were fitted with Origin software using a logistic model.

FIG. 9. Representative dose-response curves for LDDN hits. sAPPβ was determined using the BACE1 assay in 96-well format as described. Cell viability was determined at 6 (not shown) and 24 hours using Promega's Cell Titer-Glo kit, which measures ATP. sAPPβ curves were fitted with Origin software using the logistic model to determine the IC50 values.

FIG. 10. 4 compounds reduce BACE1 activity in an enzymatic assay. To further classify the small molecule hits, a FRET-based BACE1 enzymatic assay (Invitrogen) was used to identify potential direct BACE1 inhibitors. Four compounds from the LDDN series show a dose-dependent decrease in fluorescence signal, indicating that they may act on BACE1 directly.

FIG. 11. 5 LDDN compounds reduce Aβ40 in SYSY-BACEGFP-SEAPAPPwt cells. sAPPβ and 24-hour cell viability was performed as described in Chapter 4.1. Aβ40 ELISA was performed using the Aβ40 ELISA kit (BioSource) according to the manufacturer's protocol. Cell culture media was collected after 6 hours of treatment with 4 concentrations (30, 10, 3, 0.3 μM) of compound and diluted 3:10 prior to loading onto the Aβ ELISA plate. The data points for sAPPβ and Aβ were roughly superimposed. Data points represent mean±s.d. of 3 determinations.

FIG. 12A-B. Chemical structures of (A) LDN-0057228 and (B) GBR 12909.

FIG. 13. SAR studies of (A) LDN-0057228, GBR 12909, and CNS-7, a derivative of LDN-0057228; (B) 19 derivatives of LDN-0057228; and (C) 7 derivatives of LDN-0057228. The 27 structural analogs of LDN-0057228 were synthesized by medicinal chemists from the Landry Lab, and GBR 12909 was purchased from Sigma. Compounds were tested in SY5Y-BACEGFP-SEAPAPPwt cells using the cell-based BACE1 assay. Select compounds were also investigated for Aβ-lowering effect using a commercial Aβ ELISA kit (BioSource). All compounds were tested at 8 concentrations (30, 10, 3, 1, 0.3, 0.1, 0.03, and 0.01 μM), and data points were analyzed with Origin software and fitted using a logistic model for IC50 determination.

FIG. 14. Lentiviral-mediated transduction of APPsw in primary neurons. Primary cortical neurons were harvested from wild-type P0 mice and cultured according to established protocols. Lentivirus harboring human Swedish mutant APP (Lenti-APPsw) was packaged using ViraPower Lentiviral Packaging mix (Invitrogen) according to the manufacturer's protocol. DIV-14 neurons were incubated for 24 hours with primary culture media containing the indicated volume (in μl) of virus (LV-1 and LV-2 denote 2 separate batches of virus, 0 denotes no virus was used). After infection, neurons were incubated for 72 hours with primary culture media. Media was collected for Aβ40 ELISA (BioSource), and cell lysates were probed with APPCT antibody to visualize transduced full-length APP.

FIG. 15A-D. LDN-0057228 reduces Aβ40 and sAPPβ in primary neurons. DIV-14 primary cortical neurons from wild-type mice were incubated for 24 hours with primary culture media containing lentivirus harboring APPsw. After infection, neurons were incubated for 48 hours with 1:1 fresh to conditioned media to allow for APP expression. Neurons were then treated with LDN-0057228 at 20 μM for 24 hours in triplicate. A) Media was collected for Aβ40 ELISA. Raw Aβ40 values were normalized to APP to control for variations in lentiviral infection efficiency. B) Cell lysates were probed with 6E10 antibody to visualize the transduced full-length APP. C) sAPPβ from neuron culture media was immunoprecipitated with sβsw antibody and probed with LN27. D) Quantification of sAPPβ, normalized to APP. Data in A) and 110) represent mean+s.d. of 3 wells. Bands from B) and C) were quantified using ImageJ software.

FIG. 16A-B. CNS-2 and LDN-0057228 reduce Aβ40 in primary neurons. DIV-14 primary cortical neurons from wild-type mice were incubated for 24 hours with primary culture media containing lentivirus harboring APPsw. After infection, neurons were incubated for 48 hours with 1:1 fresh to conditioned media to allow for APP expression. Neurons were then treated for 24 hours with the indicated compound and concentration. A) Media was collected for Aβ40 ELISA. Raw Aβ40 values were normalized to APP to control for variations in lentiviral infection efficiency. B) Cell lysates were probed with 6E10 antibody to visualize the transduced full-length APP.

FIG. 17A-B. LDN-0057228 and CNS-2 reduce Aβ40 and sAPPβ in Tg2576 primary neurons. Primary cortical neurons were cultured from P0 APPsw transgenic pups (Tg2576). DIV-14 neurons were treated for 24 hours with LDN-0057228 or CNS-2 at the indicated concentrations. Data from two independent experiments were pooled. A) Media was collected for Aβ40 ELISA. Raw Aβ40 values were normalized to total protein to control for variations in plating density or any cytotoxicity resulting from the compounds. B) sAPPβ in the media was immunoprecipitated with sβsw antibody and visualized on Western blot with LN27.

FIG. 18. CNS-2 reduces brain total Aβ40 in Tg2576 mice. CNS-2 was dissolved in 0.9% normal saline solution with 1.9% DMSO. 12 month old Tg2576 APPsw transgenic mice were treated with 3 mg/kg CNS-2 via intraperitoneal injection at an injection volume of 20 μl per gram. 8 mice per group were treated for 9 days, 1 injection per day. Mice were sacrificed on day 9, 5 hours after the final injection. One hemisphere from each mouse was homogenized, and the homogenate was processed for formic acid extraction of plaque Aβ. After formic acid extraction, total Aβ40 was measured via ELISA kit (BioSource) and normalized to total protein.

FIG. 19A-H. Small molecule compounds of the invention.

FIG. 20A-H. Dose-related BACE1 inhibition and cytotoxicity of compounds depicted in FIG. 19.

FIG. 21A-L. Dose-response curves of levels of sAPPβ; cytotoxicity, and BACE1 binding (by FRET assay) of certain compounds of the invention depicted in FIG. 19.

### 5. DETAILED DESCRIPTION

The present invention is based on the discovery of certain compounds that inhibit BACE1 enzymatic activity and decrease the level of APP metabolism through the β-secretase metabolic pathway. In light of the role APP metabolism plays in connection with neurodegenerative conditions, such as, but not limited to, Alzheimer's Disease, the compounds of the instant invention can be used to inhibit BACE1 activity and thereby ameliorate neurodegenerative conditions.

For clarity and not by way of limitation, this detailed description is divided into the following sub-portions:

(i) definitions;

(ii) BACE1 inhibitors and synthesis schemes;

(iii) methods of treatment; and

(iv) pharmaceutical compositions.

### 5.1 Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “BACE1” refers to a polypeptide which mediates the cleavage of APP in the β-amyloidgenic pathway, producing an sAPPβ ectodomain APP metabolite, which is released into the extracellular space, and an intracellular C-terminal fragment (CTF). In one non-limiting embodiment, the BACE1 is a human BACE1. The BACE1 is preferably encoded by the Homo sapiens beta-site APP-cleaving enzyme 1 (BACE1) gene (GenBank accession numbers NM_{—}012104, NM_{—}138972, NM_{—}138971, or NM_{—}138973), or any nucleic acid which encodes a human BACE1 polypeptide. Alternatively, BACE1 can be encoded by any nucleic acid molecule exhibiting at least 50%, at least 60%, at least 70%, at least 80%, at least 90%, at least 95%, or up to 100% homology to a BACE1 gene (as determined by standard software, e.g. BLAST or FASTA), and any sequences which hybridize under stringent conditions to these sequences which retain BACE1 activity, where stringent conditions are as described in U.S. Published Patent Application US20030082140, which is hereby incorporated by reference in its entirety and for all purposes.

In other non-limiting embodiments, a BACE1 of the invention may be characterized as having an amino acid sequence described by GenBank accession numbers: NP_{—}036236, NP_{—}620428, NP_{—}620427 and NP_{—}620429, or any other amino acid sequence at least 90%, or at least 95% homologous thereto, which retains BACE1 activity.

The terms “APP” or “amyloid precursor protein” refers to a substrate of BACE1 which may be metabolized into an ectodomain sAPPβ fragment and a C-terminal fragment (CTF). In one embodiment, APP is an integral membrane protein expressed in many tissues and concentrated in, for example, the synapses of neurons. In one non-limiting embodiment, APP is a human APP, for example, Homo sapiens amyloid beta (A4) precursor protein (APP) encoded by an APP gene (e.g., GenBank Accession numbers: NM_{—}201414, NM_{—}201413, or NM_{—}000484), or any nucleic acid that encodes a human APP polypeptide. Alternatively, APP can be encoded by any nucleic acid molecule exhibiting at least 50%, at least 60%, at least 70%, at least 80%, at least 90%, at least 95%, or up to 100% homology to any one of the APP genes (as determined by standard software, e.g. BLAST or FASTA), and any sequences which hybridize under stringent conditions to these sequences.

In other non-limiting embodiments, APP may be characterized as comprising an amino acid sequence described by GenBank accession numbers: NP_{—}958817, NP_{—}958816, or NP_{—}000475, or any other amino acid sequence at least 90% or at least 95% homologous thereto and is cleavable by a human BACE1 protein. In non-limiting embodiments APP may be comprised in a fusion protein.

The BACE1 or APP may be a recombinant BACE1 or APP polypeptide encoded by a recombinant nucleic acid, for example, a recombinant DNA molecule, or may be of natural origin.

According to the invention, a “subject” or “patient” is a human or non-human animal. Although the animal subject is preferably a human, the compounds and compositions of the invention have application in veterinary medicine as well, e.g., for the treatment of domesticated species such as canine, feline, and various other pets; farm animal species such as bovine, equine, ovine, caprine, porcine, etc.; wild animals, e.g., in the wild or in a zoological garden; and avian species, such as chickens, turkeys, quail, songbirds, etc.

The term ‘alkyl’ refers to a straight or branched C₁-C₂₀, preferably C₁-C₅, hydrocarbon group consisting solely of carbon and hydrogen atoms, containing no unsaturation, and which is attached to the rest of the molecule by a single bond, e.g., methyl, ethyl, n-propyl, 1-methylethyl (isopropyl), n-butyl, n-pentyl, 1,1-dimethylethyl (t-butyl).

The term “alkenyl” refers to a C₂-C₂₀, preferably C₁-C₅, aliphatic hydrocarbon group containing at least one carbon-carbon double bond and which may be a straight or branched chain, e.g., ethenyl, 1-propenyl, 2-propenyl iso-propenyl, 2-methyl-l-propenyl, 1-butenyl, 2-butenyl.

The term “cycloalkyl” denotes an unsaturated, non-aromatic mono- or multicyclic hydrocarbon ring system such as cyclopropyl, cyclobutyl, cyclopentyl, cyclohexyl. Examples of multicyclic cycloalkyl groups include perhydronapththyl, adamantyl and norbornyl groups bridged cyclic group or sprirobicyclic groups, e.g., spiro (4,4) non-2-yl.

The term “aryl” refers to aromatic radicals having in the range of about 6 to about 14 carbon atoms such as phenyl, naphthyl, tetrahydronapthyl, indanyl, biphenyl.

The term “heterocyclic” refers to a stable 3- to 15-membered ring radical which consists of carbon atoms and one or more, for example, from one to five, heteroatoms selected from the group consisting of nitrogen, oxygen and sulfur. For purposes of this invention, the heterocyclic ring radical may be a monocyclic or bicyclic ring system, which may include fused or bridged ring systems, and the nitrogen, carbon, oxygen or sulfur atoms in the heterocyclic ring radical may be optionally oxidized to various oxidation states. In addition, a nitrogen atom, where present, may be optionally quaternized; and the ring radical may be partially or fully saturated (L e., heteroaromatic or heteroaryl aromatic).

The heterocyclic ring radical may be attached to the main structure at any heteroatom or carbon atom that results in the creation of a stable structure.

The term “heteroaryl” refers to a heterocyclic ring wherein the ring is aromatic.

The substituents in the ‘substituted alkyl’, ‘substituted alkenyl’, ‘substituted cycloalkyl’, ‘substituted aryl,’ substituted heteroaryl’ ‘substituted alkoxy,’ ‘substituted aryloxy,’ ‘substituted alkylthiol,’ and ‘substituted arylthiol’ may be the same or different, with one or more selected from the groups hydrogen, halogen, acetyl, nitro, oxo (═O), CF₃, NH₂, OCH₃, or optionally substituted groups selected from alkyl, alkoxy and aryl.

The term “halogen” refers to fluorine, chlorine, bromine and iodine.

### 5.2 BACE1 Inhibitors and Synthesis Schemes

The present invention provides for compounds that inhibit the production of sAPPβ and Aβ.

In certain non-limiting embodiments, the invention provides for compounds of the following Formula I:

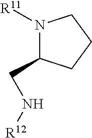

wherein R¹¹ and R¹² are independently selected for each occurrence from the group consisting of substituted or unsubstituted alkyl, cycloalkyl, aryl, heteroaryl and alkenyl.

In other non-limiting embodiments, R¹¹ is independently selected for each occurrence from the group consisting of ethyl and:

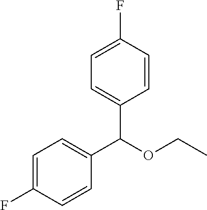

In other non-limiting embodiments, R¹² is independently selected for each occurrence from the group consisting of hydrogen, methyl, COCH₃ and:

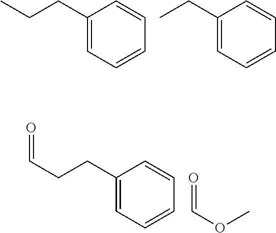

In other non-limiting embodiments, the invention provides for compounds of the following Formula II:

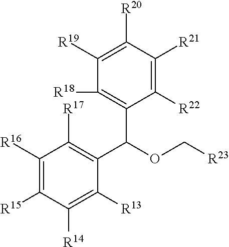

wherein R²³ is selected from the group consisting of substituted or unsubstituted alkyl, cycloalkyl, aryl, heteroaryl and alkenyl, and wherein R¹³-R²² are independently selected for each occurrence from the group consisting of hydrogen, halogen, alkyl, aryl, CN, alkoxy, aryloxy, NO₂, alkylthio, and arylthio. In certain embodiments, R¹³-R²² are independently selected for each occurrence from the group consisting of hydrogen and halogen. In certain embodiments, R¹³-R¹⁴, R¹⁶-R¹⁹, and R²¹-R²² are hydrogen and R¹⁵ and R²⁰ independently selected for each occurrence from the group consisting of hydrogen and halogen. In certain embodiments, R¹³-R¹⁴, R¹⁶-R¹⁹, and R²¹-R²² are hydrogen and R¹⁵ and R²⁰ independently selected for each occurrence from the group consisting of hydrogen, F, Cl, and Br. In certain embodiments, R¹³-R¹⁴, R¹⁶-R¹⁹, and R²¹-R²² are hydrogen and R¹⁵ and R²⁰ independently selected for each occurrence from the group consisting of F, Cl, and Br. In certain embodiments, R¹³-R¹⁴, R¹⁶-R¹⁹, and R²¹-R²² are hydrogen and R¹⁵ and R²⁰ are F.

In one embodiment, the compound defined by Formula II is:

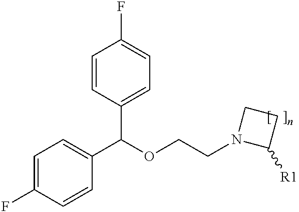

| | | | IC₅₀(sAPPβ) | IC₅₀(Aβ40) |
|---|---|---|---|---|
| Compound | n | R1 | (μM) | (μM) |
| CNS-1 | 2 | (S)-CH₂NH(CH₂)₃Ph | 10.5 | |
| CNS-2 | 2 | (S)-CH₂NHCO(CH₂)₂Ph | 8.9 | 5.0 |
| CNS-3 | 2 | (S)-CH₂NHCH₃ | 11.5 | 11.5 |
| CNS-4 | 2 | (S)-CH₂NHCOOCH₃ | 23.3 | |
| CNS-5 | 2 | (S)-CH₂NHCOCH₃ | 20.8 | |
| CNS-6 | 2 | (S)-CH₂NH₂ | 27.9 | |
| CNS-8 | 2 | (R)-CH₂NH(CH₂)₃Ph | 5.7 | |
| CNS-9 | 2 | (R)-CH₂NHCO(CH₂)₂Ph | 20.4 | |
| CNS-10 | 2 | (R)-CH₂NH₂ | 38.3 | |
| CNS-11 | 2 | (R)-CONH₂ | inactive | |
| CNS-12 | 3 | (S)-CH₂NH₂ | >50 | |
| CNS-13 | 3 | (S)-CH₂NH(CH₂)₃Ph | 9.0 | |
| CNS-14 | 3 | (S)-CH₂NHCO(CH₂)₂Ph | 14.0 | |
| CNS-15 | 3 | (S)-CONH₂ | inactive | |
| CNS-16 | 2 | (S)-CONH₂ | inactive | |
| CNS-17 | 1 | (S)-CONH₂ | inactive | |
| CNS-18 | 1 | (S)-CH₂NH₂ | >50 | |
| CNS-19 | 1 | (S)-CH₂NHCO(CH₂)₂Ph | inactive | |
| CNS-20 | 1 | (S)-CH₂NH(CH₂)₃Ph | inactive | |

In other embodiments, the compound defined by Formula II is:

C

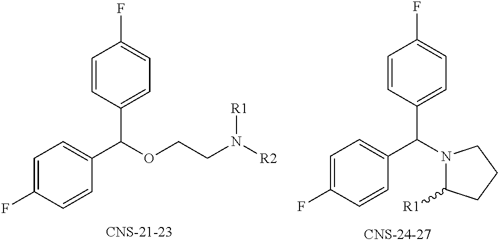

| | | | IC50(sAPPβ) |
|---|---|---|---|
| Compound | R1 | R2 | (μM) |
| CNS-21 | H | CH₂CH₂NHCO(CH₂)₂Ph | inactive |
| CNS-22 | H | CH₂CH₂NH(CH₂)₃Ph | inactive |
| CNS-23 | CH₂CH₂NHCO(CH₂)₂Ph | CH₂CH₂OCH(4-F—Ph)₂ | inactive |
| CNS-24 | (S)-CONH₂ | | inactive |
| CNS-25 | (S)-CH₂NH₂ | | 31.4 |
| CNS-26 | (S)-CH₂NHCO(CH₂)₂Ph | | 32.1 |
| CNS-27 | (S)-CH₂NH(CH₂)₃Ph | | 10.2 |

In one preferred embodiment, the compound defined by Formula II is:

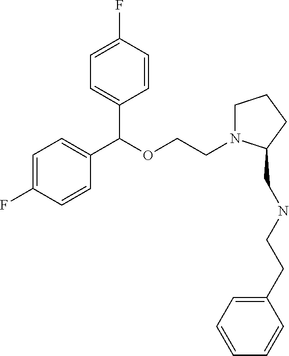

In another preferred embodiment, the compound defined by Formula II is:

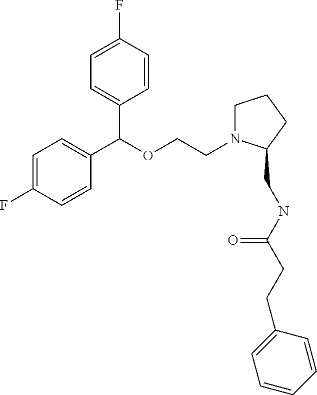

In other non-limiting embodiments, the invention provides for compounds of the following Formula III:

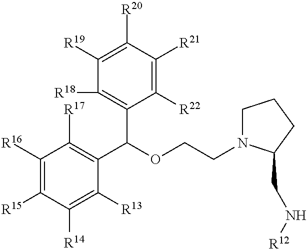

wherein R¹² is selected from the group consisting of substituted or unsubstituted alkyl, cycloalkyl, aryl, heteroaryl and alkenyl, and wherein R¹³-R²² are independently selected for each occurrence from the group consisting of hydrogen, halogen, alkyl, aryl, CN, alkoxy, aryloxy, NO₂, alkylthio, and arylthio. In certain embodiments, R¹³-R²² are independently selected for each occurrence from the group consisting of hydrogen and halogen. In certain embodiments, R¹³-R¹⁴, R¹⁶-R¹⁹, and R²¹-R²² are hydrogen and R¹⁵ and R²⁰ independently selected for each occurrence from the group consisting of hydrogen and halogen. In certain embodiments, R¹³-R¹⁴, R¹⁶-R¹⁹, and R²¹-R²² are hydrogen and R¹⁵ and R²⁰ independently selected for each occurrence from the group consisting of hydrogen, F, Cl, and Br. In certain embodiments, R¹³-R¹⁴, R¹⁶-R¹⁹, and R²¹-R²² are hydrogen and R¹⁵ and R²⁰ independently selected for each occurrence from the group consisting of F, Cl, and Br. In certain embodiments, R¹³-R¹⁴, R¹⁶-R¹⁹, and R²¹-R²² are hydrogen and R¹³ and R²⁰ are F.

In another non-limiting embodiment, the compounds of Formulas I, II and III may be synthesized by any means known in the art. For example, compounds of Formulas I, II and III may be synthesized according to the following scheme:

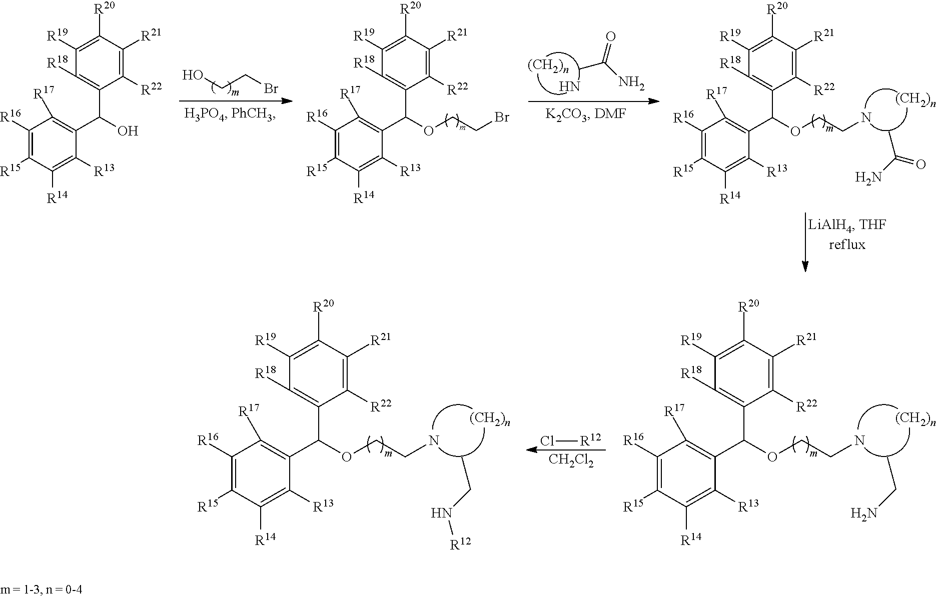

wherein R¹³-R²² are independently selected for each occurrence from the group consisting of hydrogen, halogen, alkyl, aryl, CN, alkoxy, aryloxy, NO₂, alkylthio, and arylthio. In certain embodiments, R¹³-R²² are independently selected for each occurrence from the group consisting 15R R′⁶-R¹⁹, of hydrogen and halogen. In certain embodiments, R¹³-R¹⁴, R¹⁶-R¹⁹, and R²¹-R²² are hydrogen and R¹⁵ and R²⁰ independently selected for each occurrence from the group consisting of hydrogen and halogen. In certain embodiments, R¹³-R¹⁴, R¹⁶-R¹⁹, and R²¹-R²² are hydrogen and R¹⁵ and R²⁰ independently selected for each occurrence from the group consisting of hydrogen, F, Cl, and Br. In certain embodiments, R¹³-R¹⁴, R¹⁶-R¹⁹, and R²¹-R²² are hydrogen and R¹⁵ and R²⁰ independently selected for each occurrence from the group consisting of F, Cl, and Br. In certain embodiments, R¹³-R¹⁴, R¹⁶-R¹⁹, and R²¹-R²² are hydrogen and R¹⁵ and R²⁰ are F.

In another non-limiting embodiment, the compound of Formula III may be synthesized according to the following scheme:

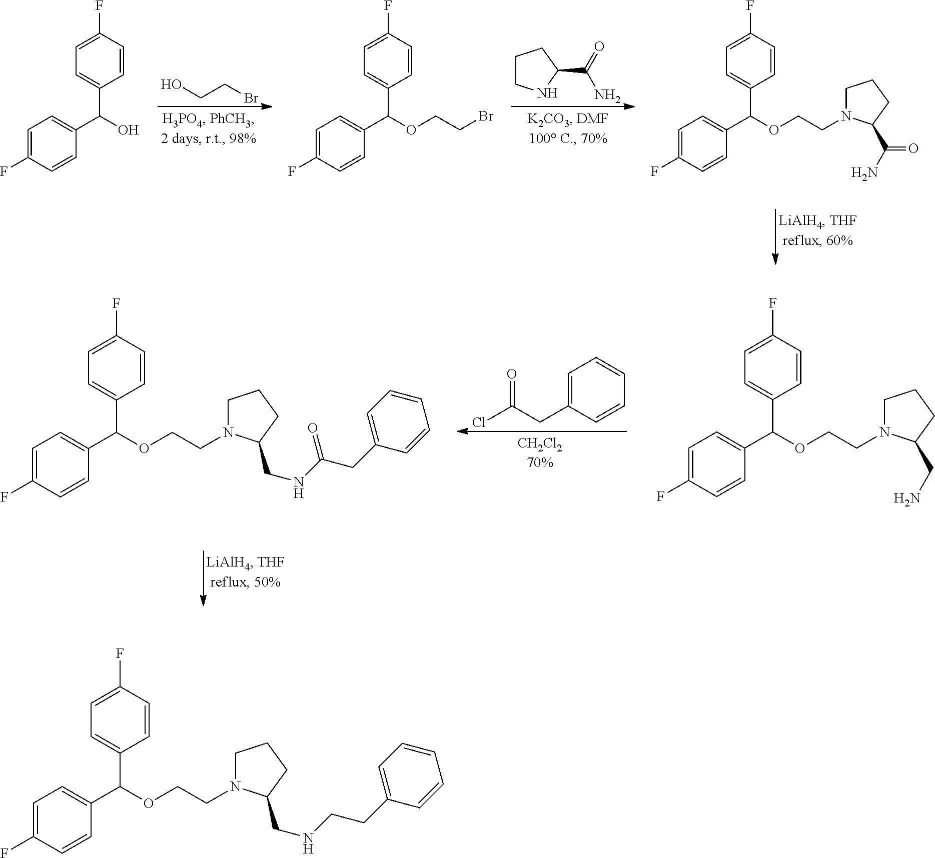

In other embodiments, the invention provides for compounds of the following Formula IV:

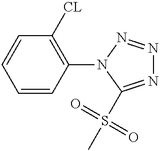

In other embodiments, the invention provides for compounds of the following Formula V

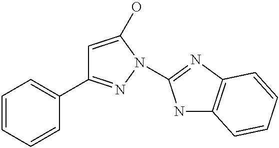

In other embodiments, the invention provides for compounds of the following Formula VI:

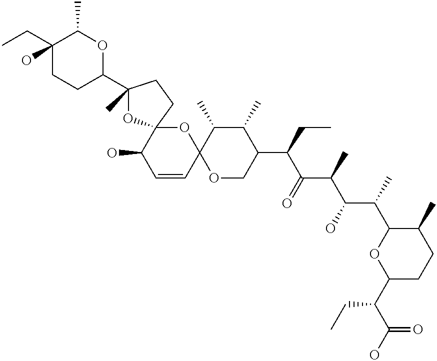

In other embodiments, the invention provides for compounds of the following

Formula VII:

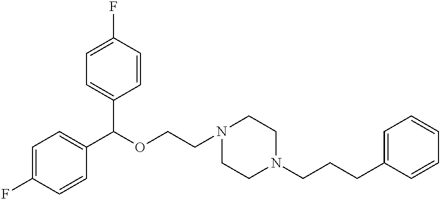

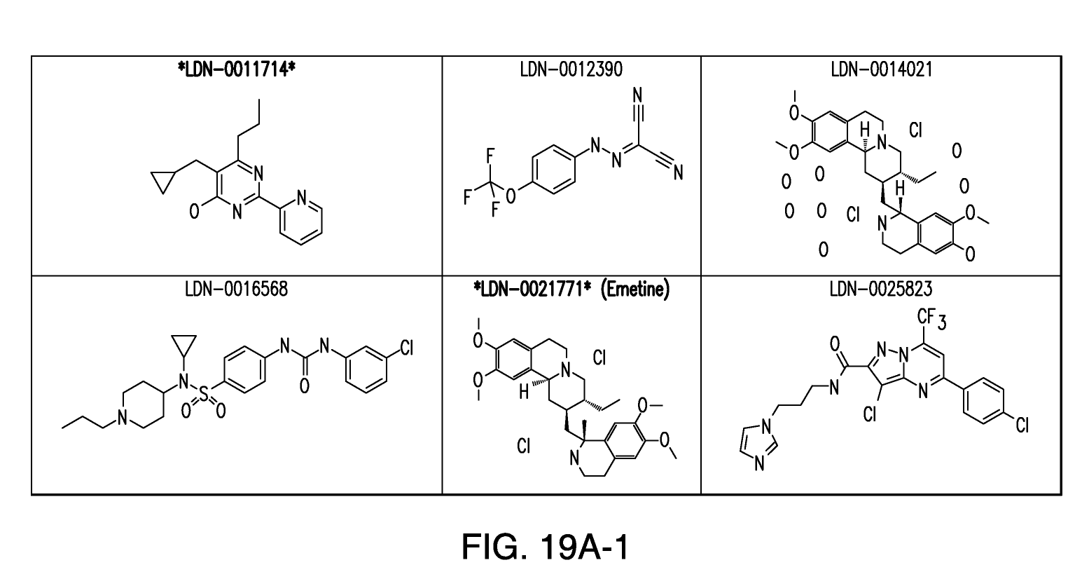

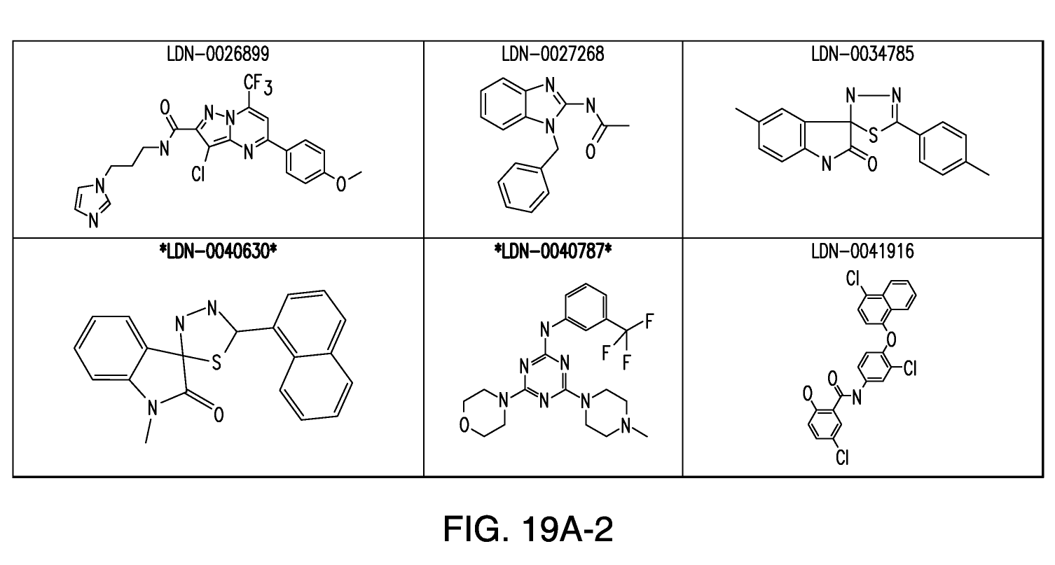

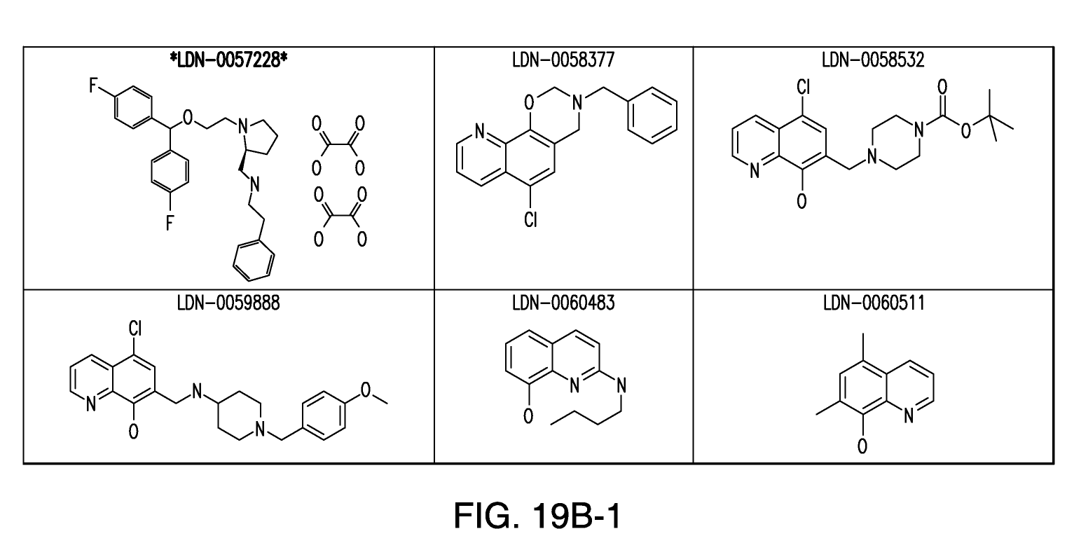

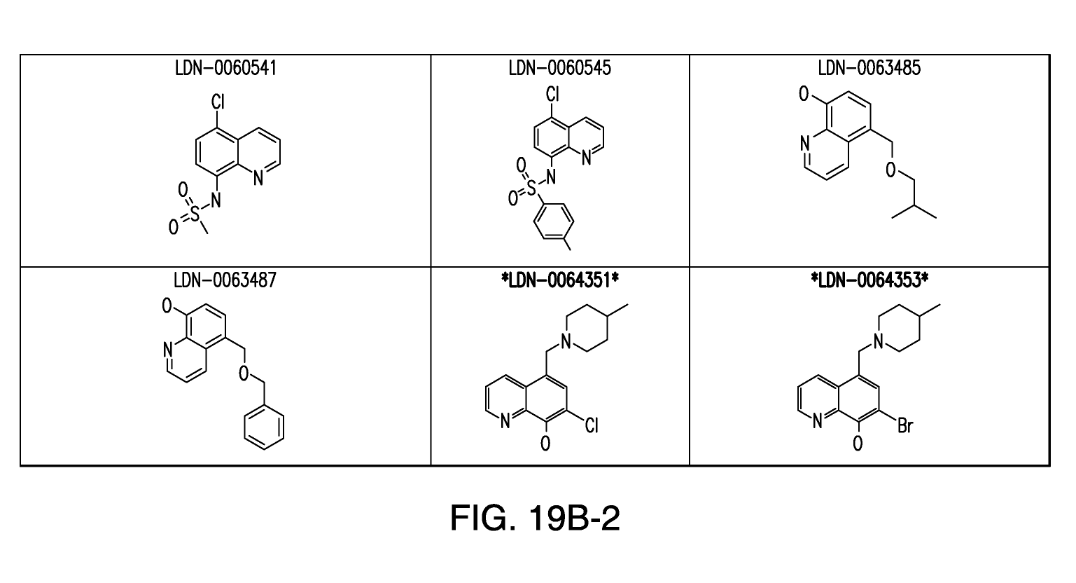

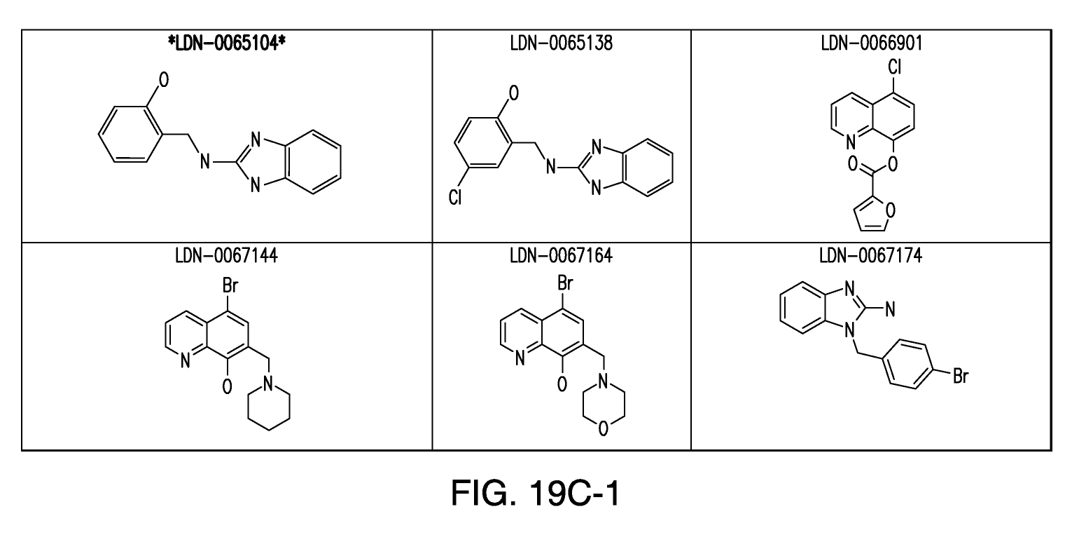

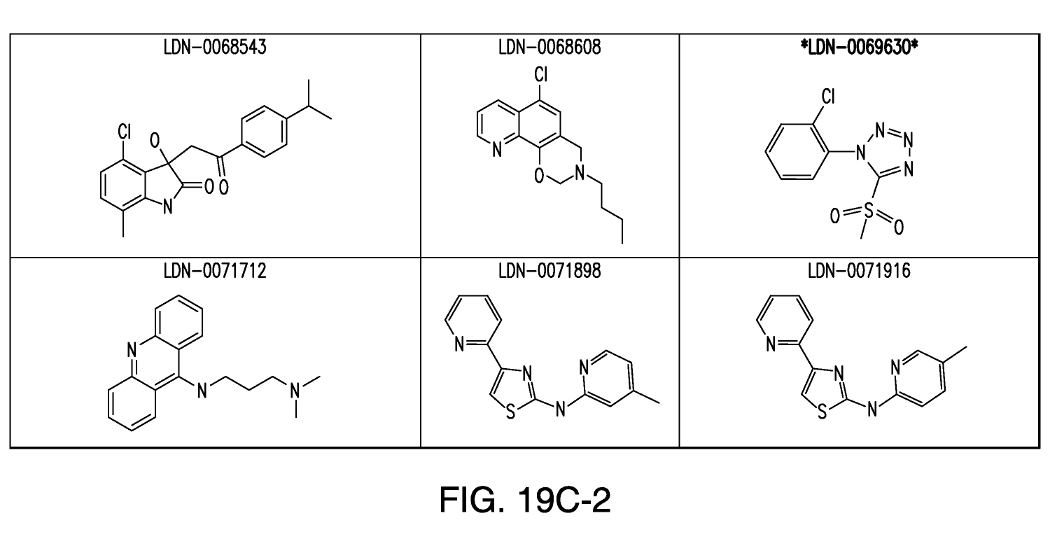

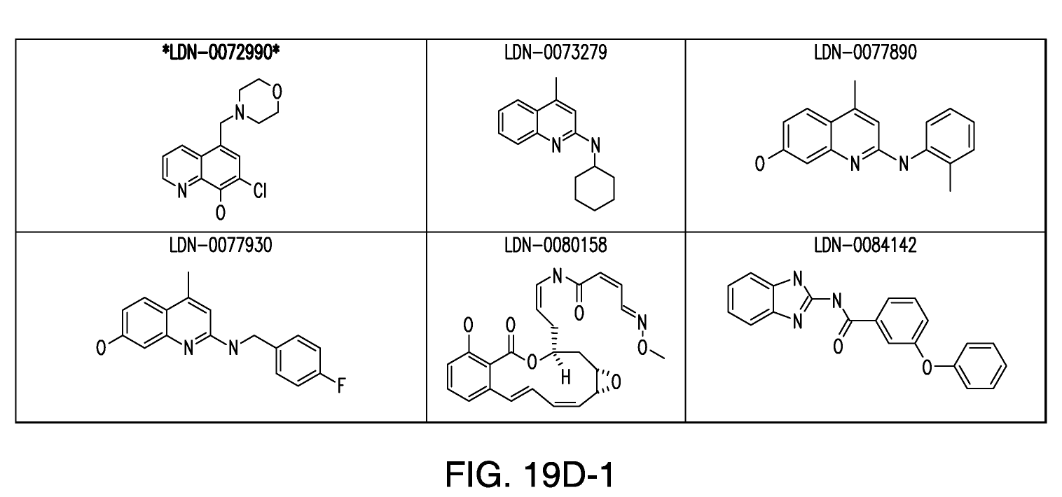

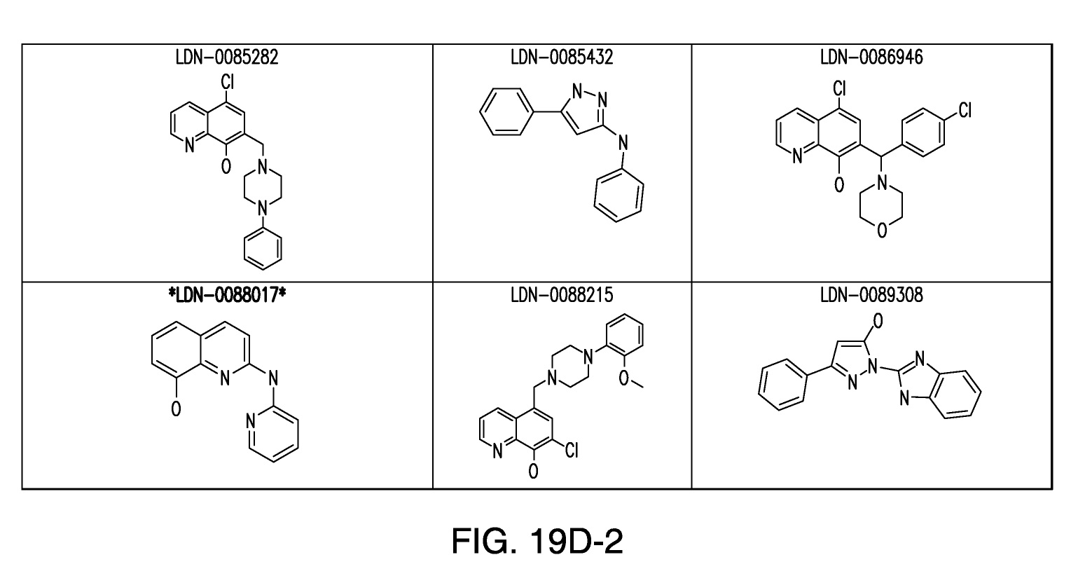

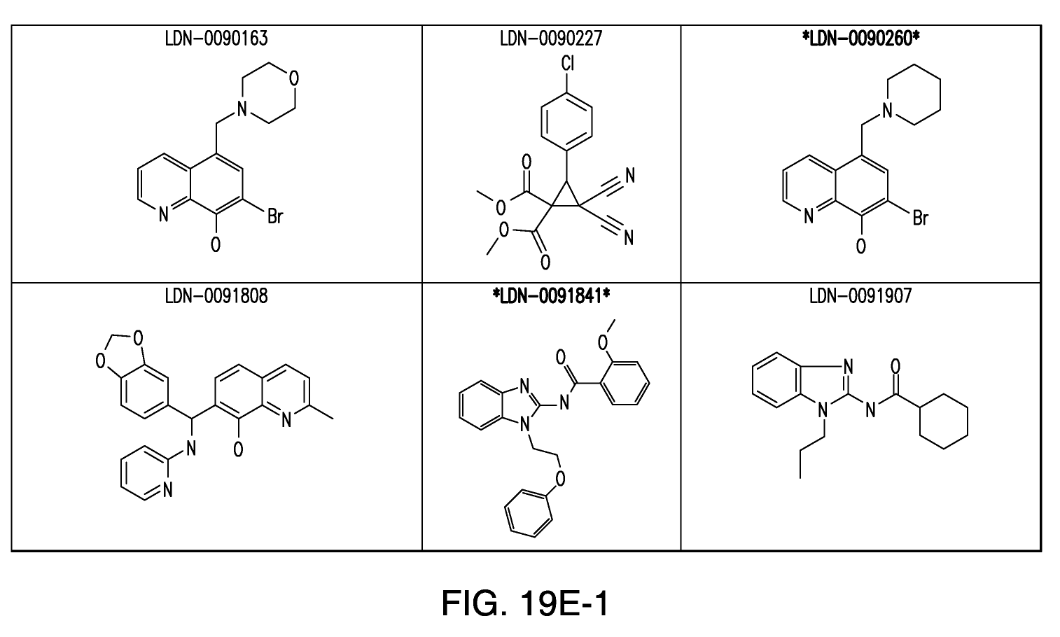

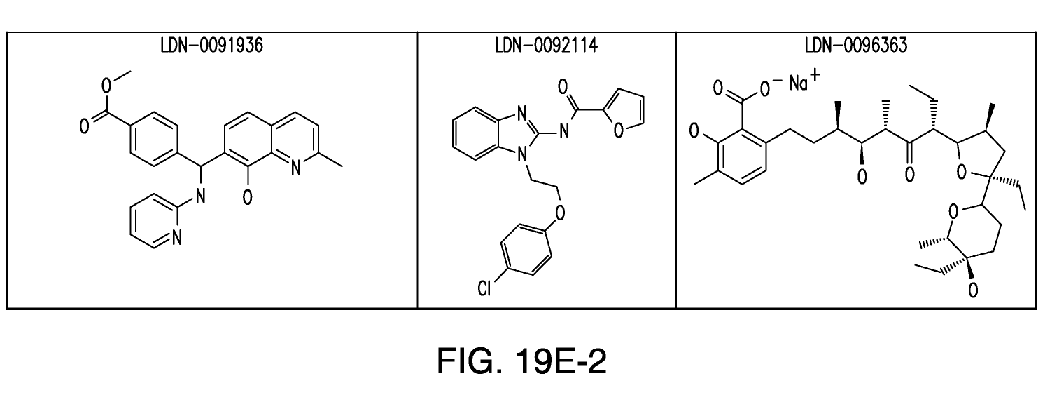

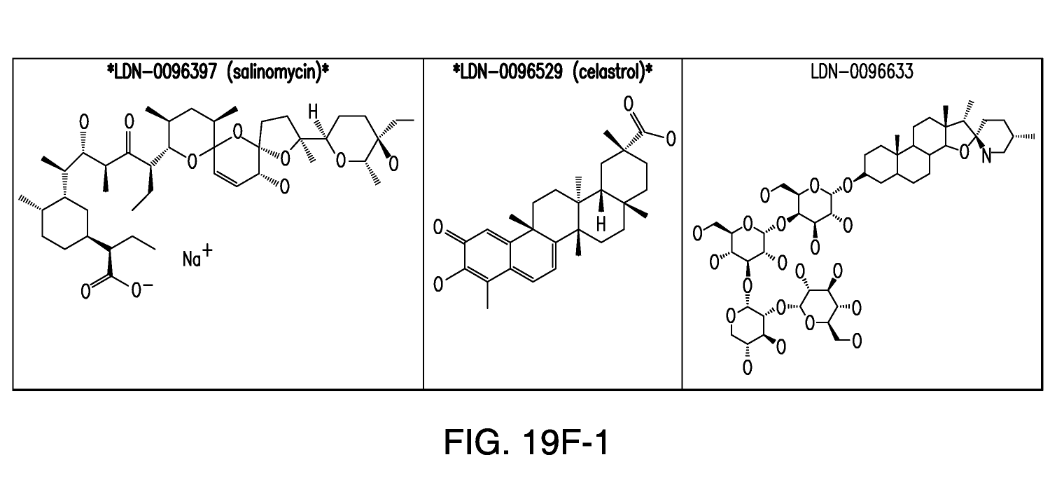

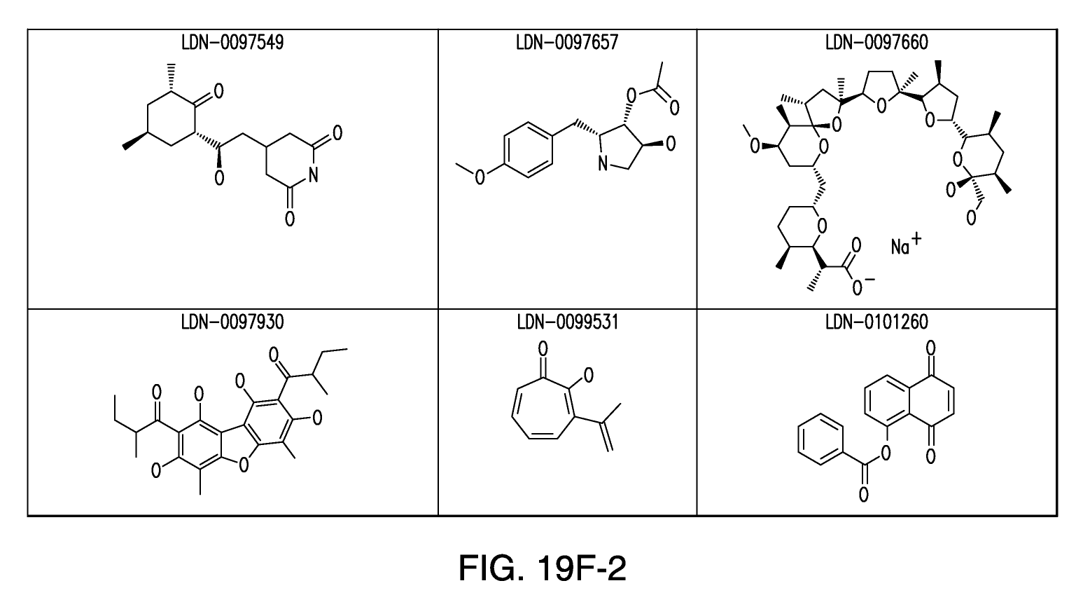

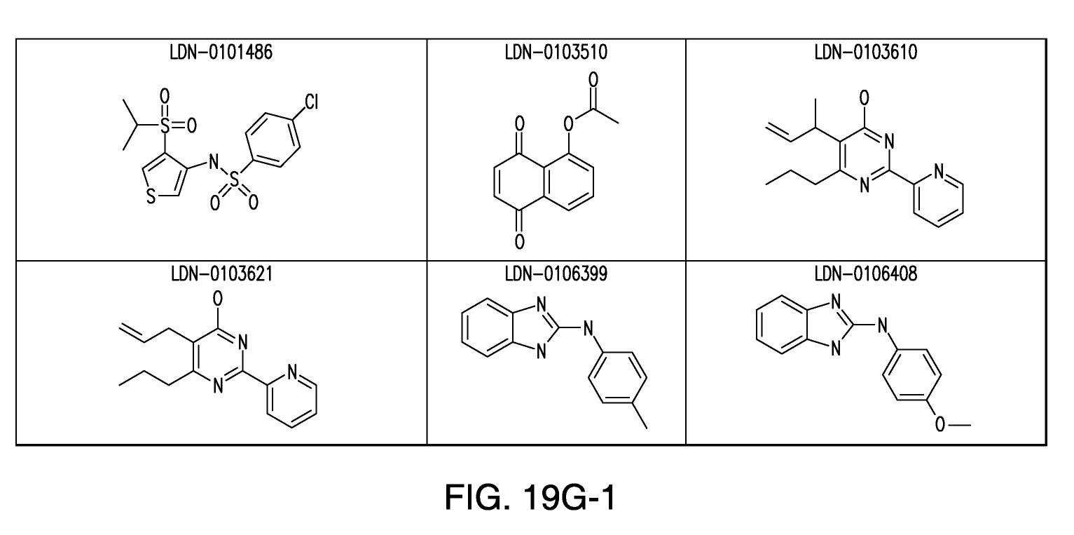

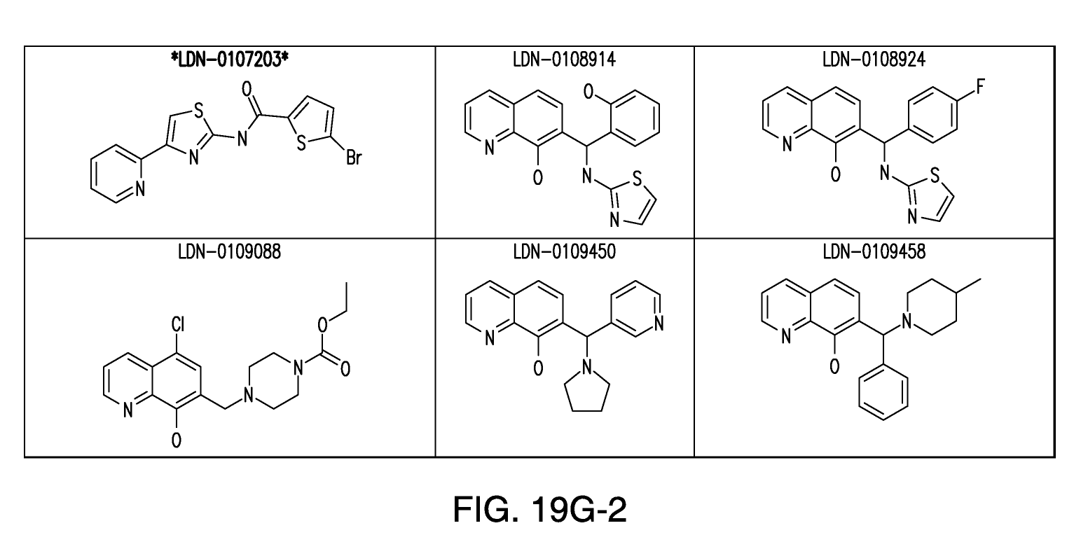

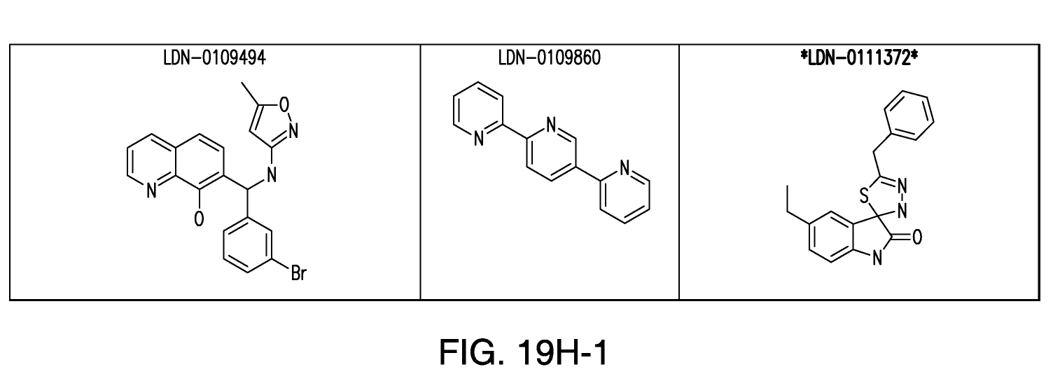

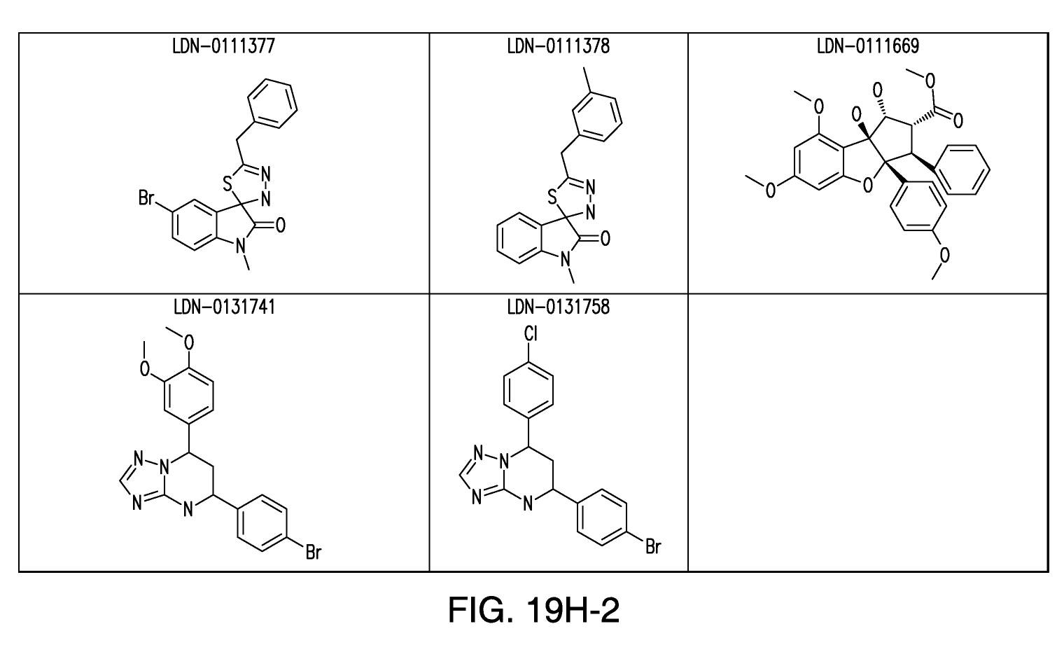

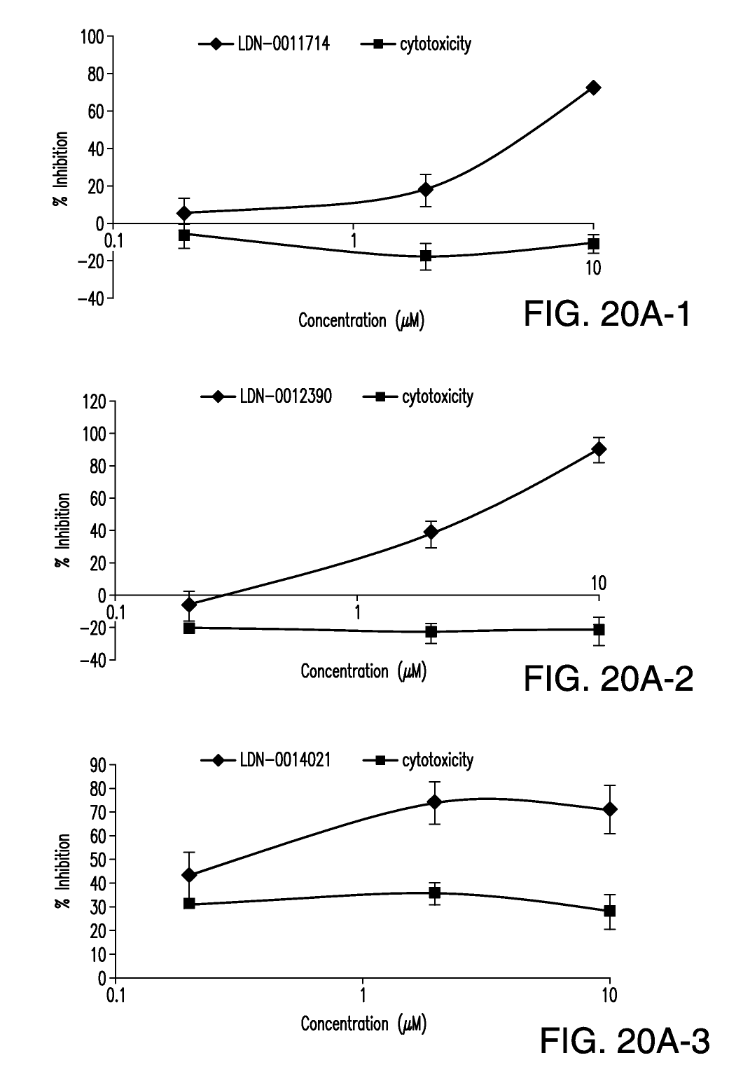

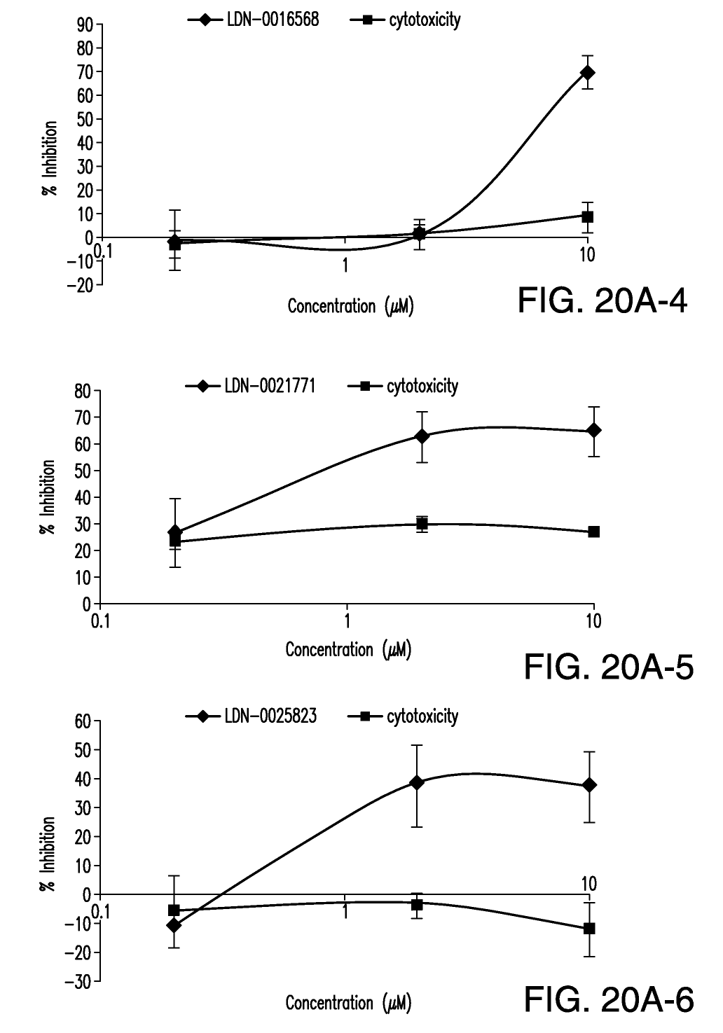

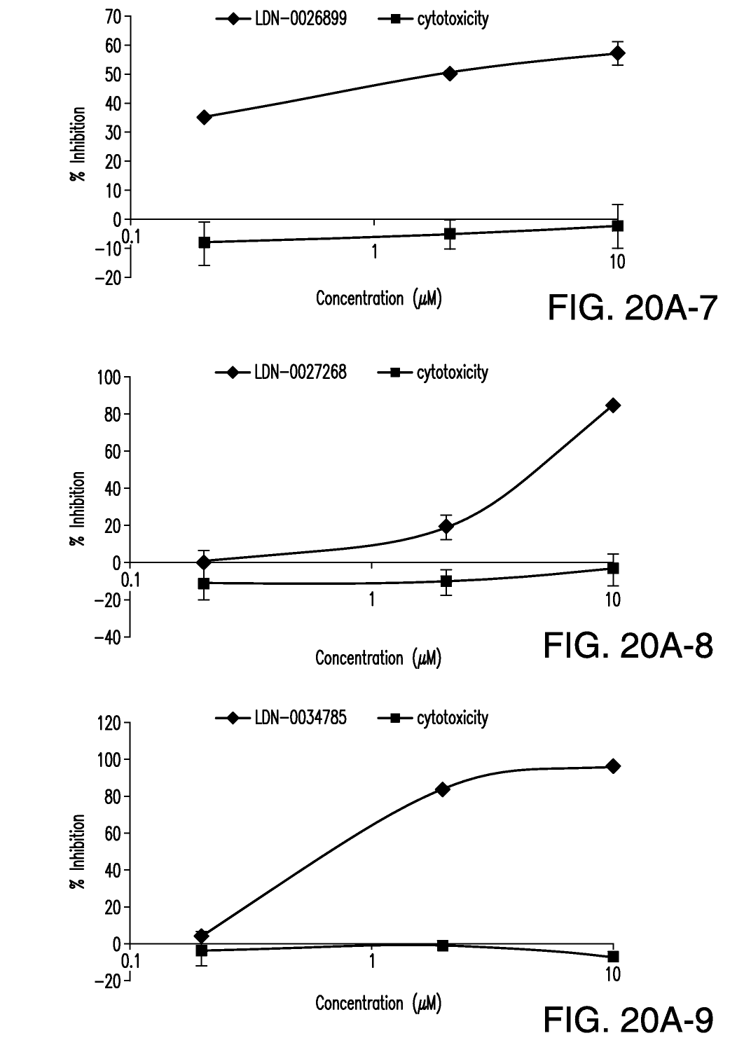

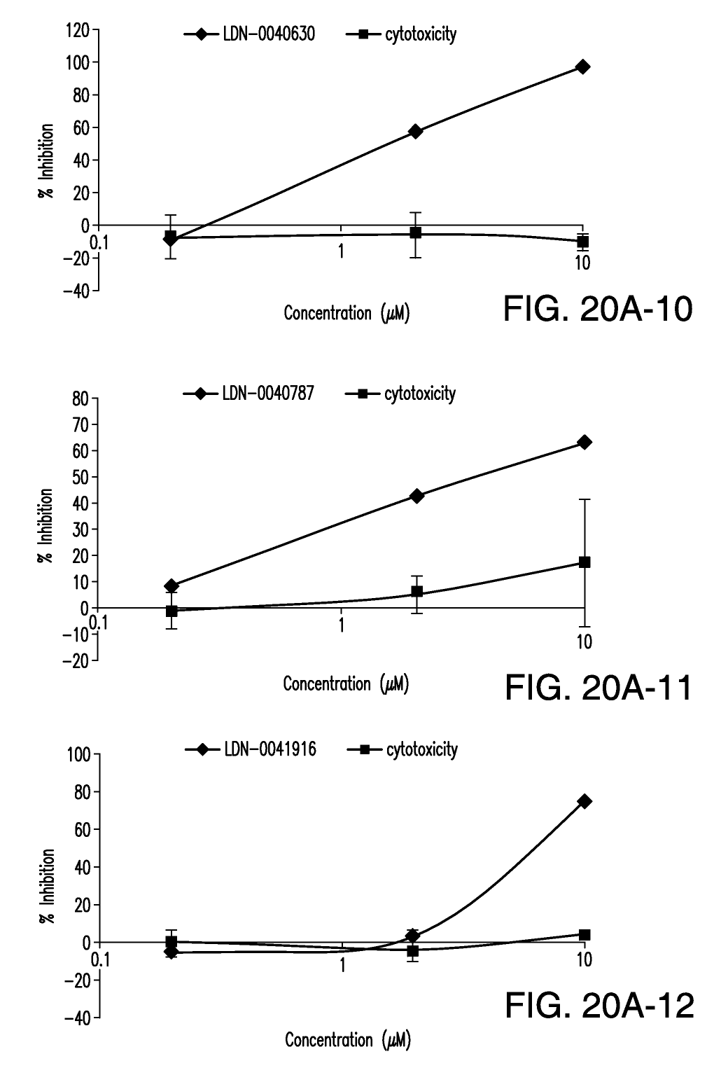

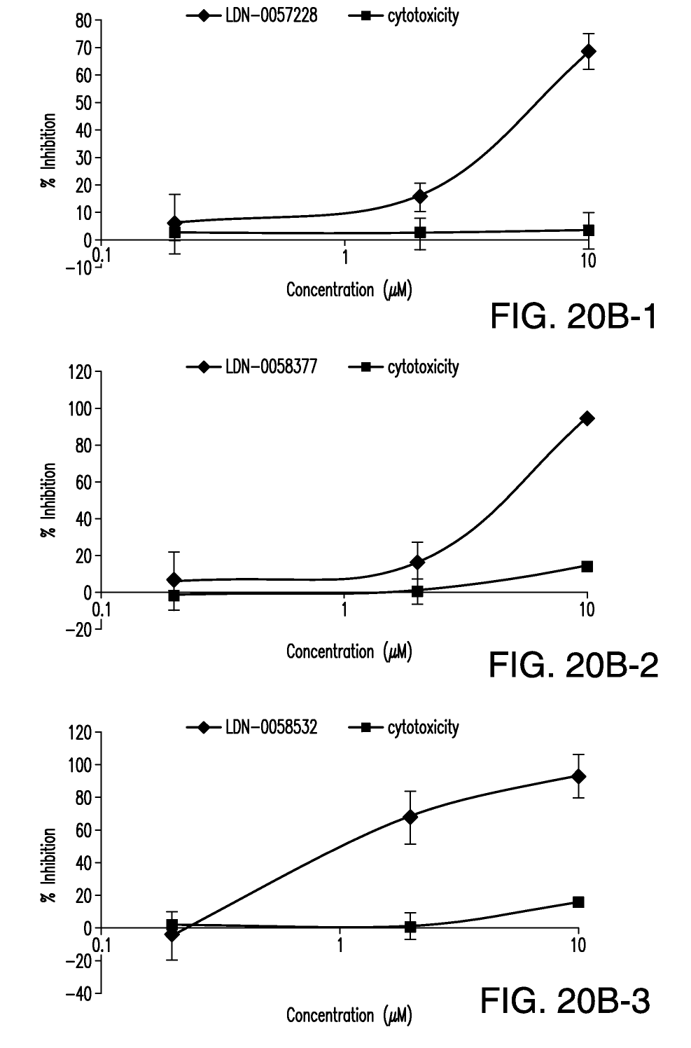

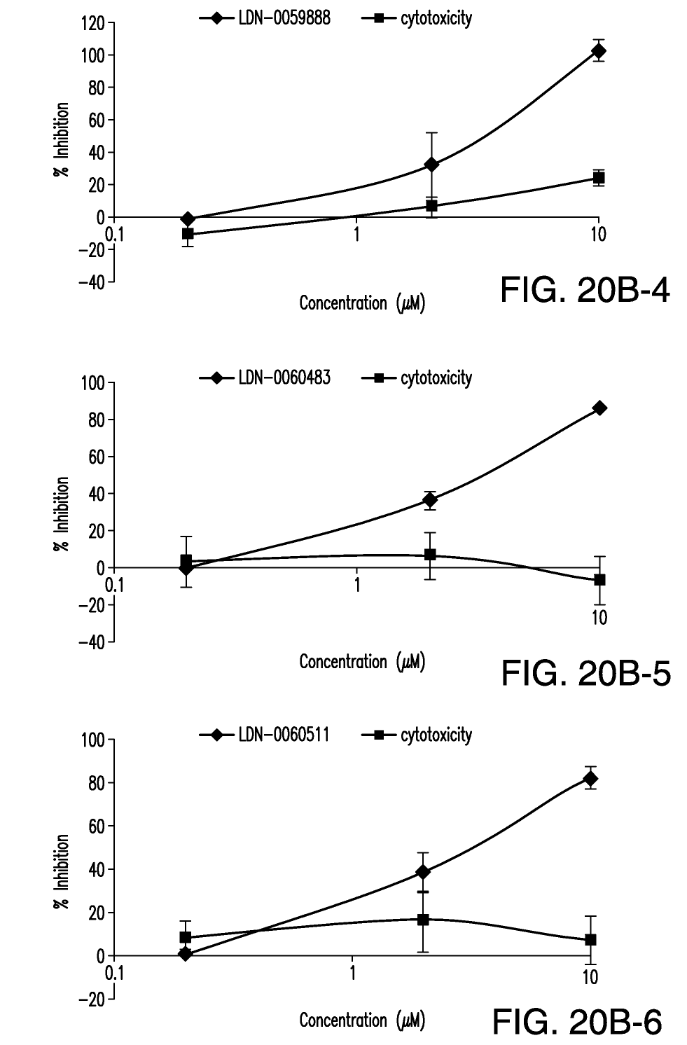

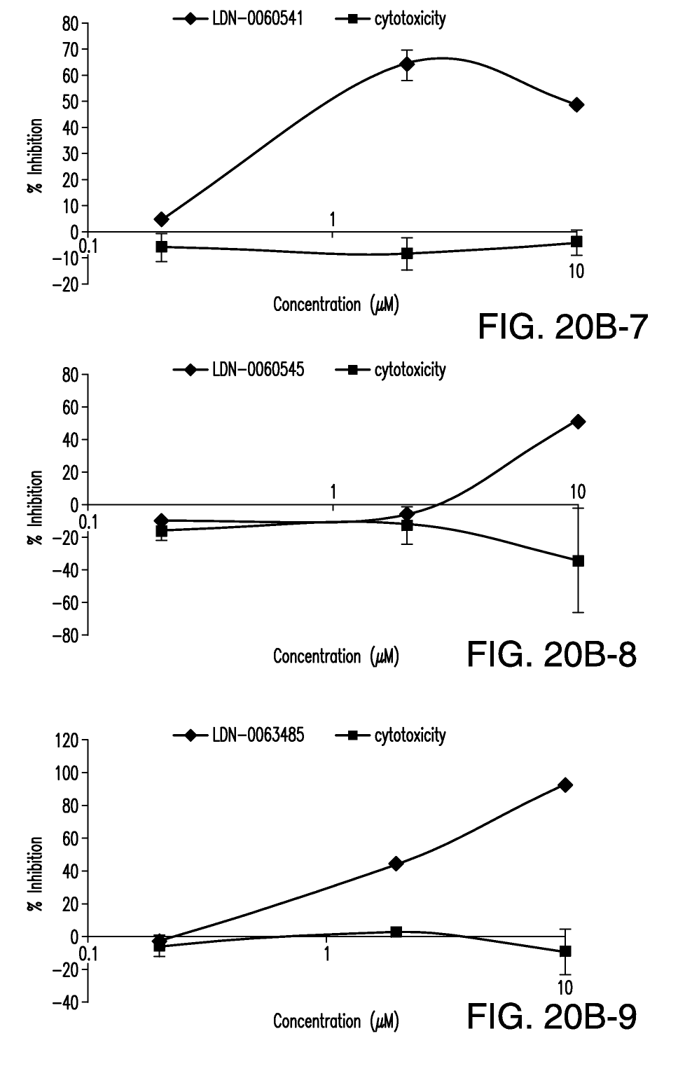

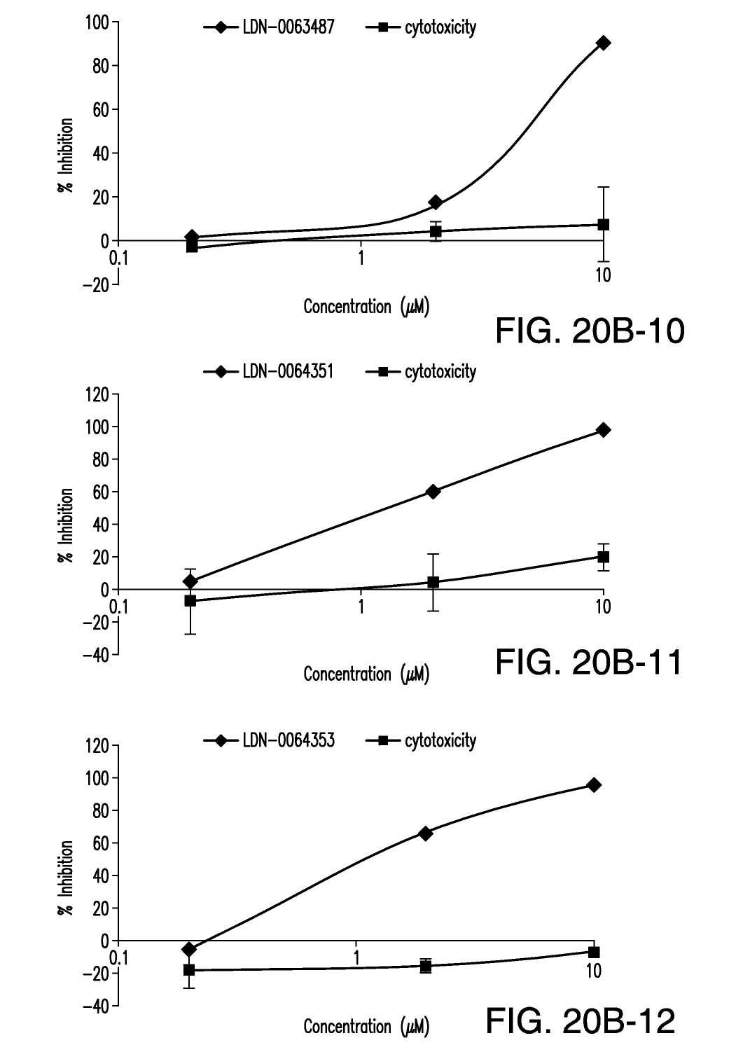

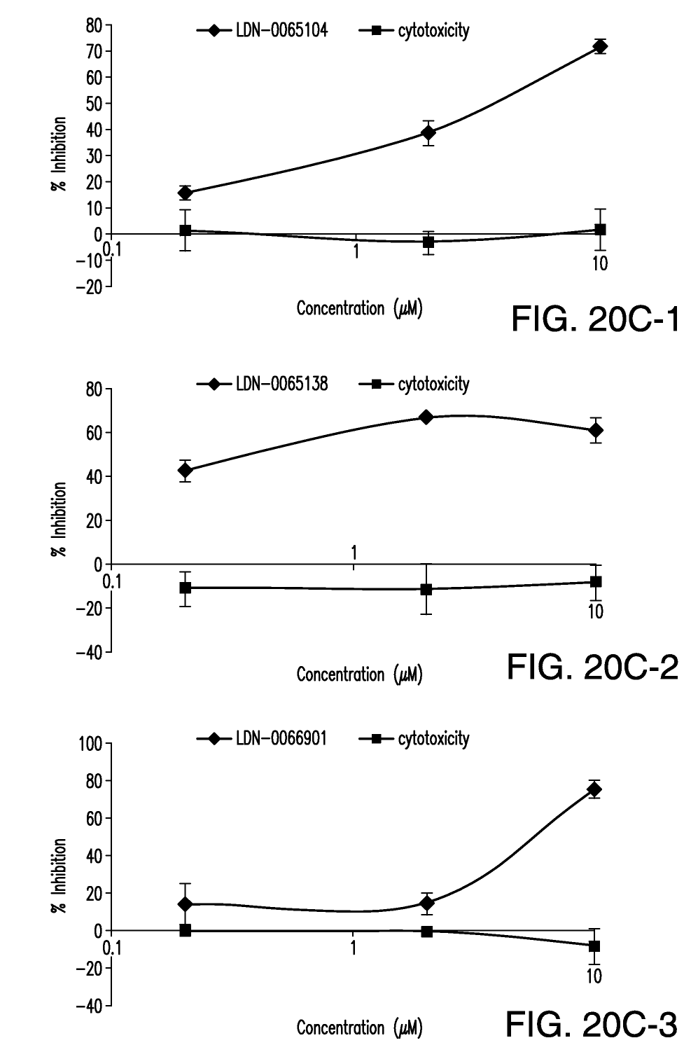

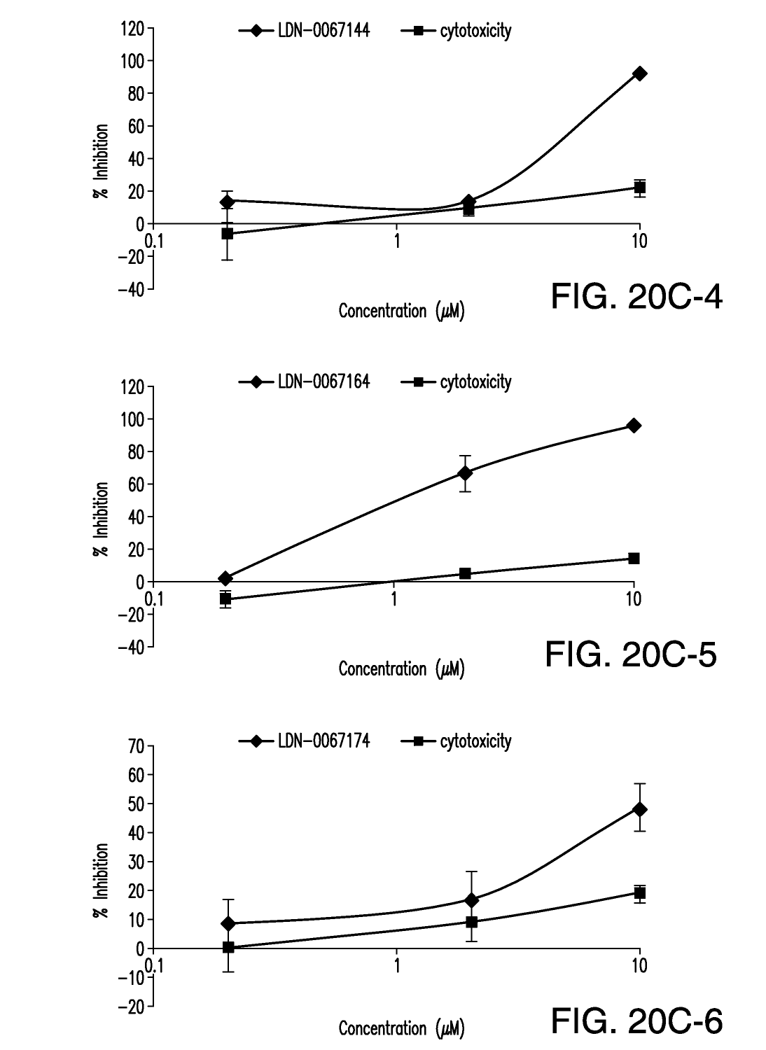

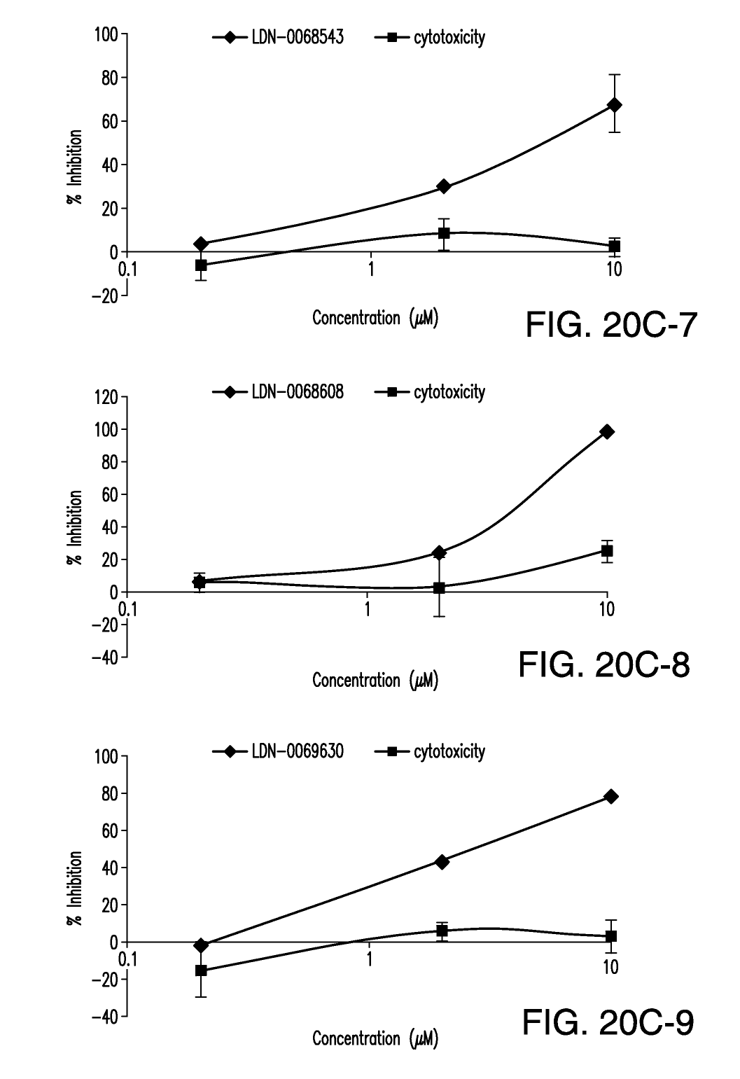

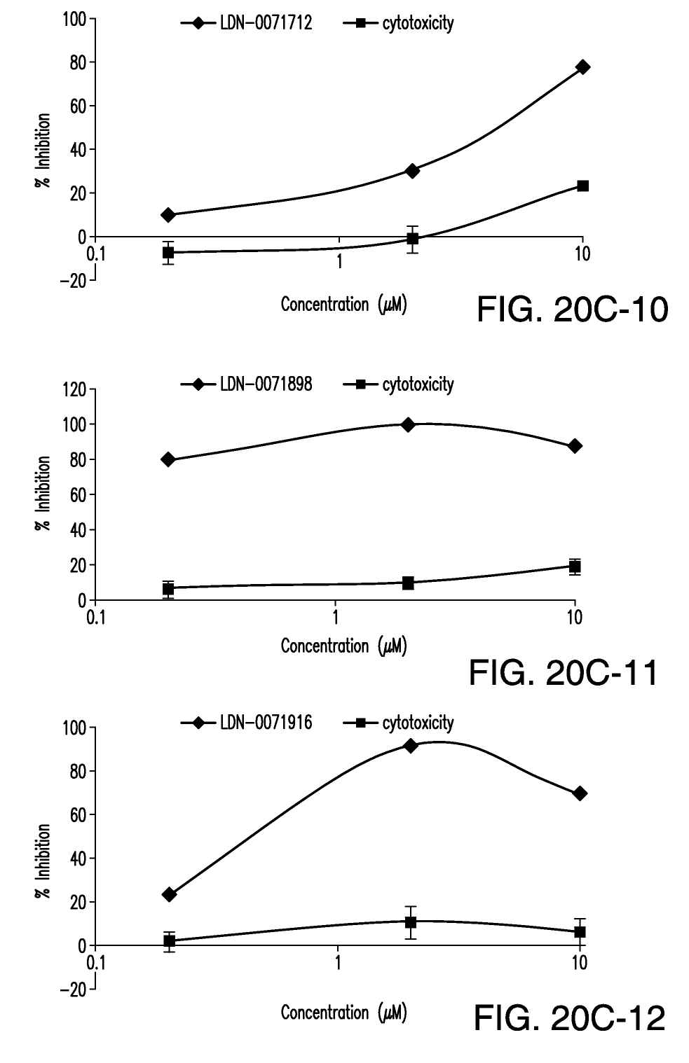

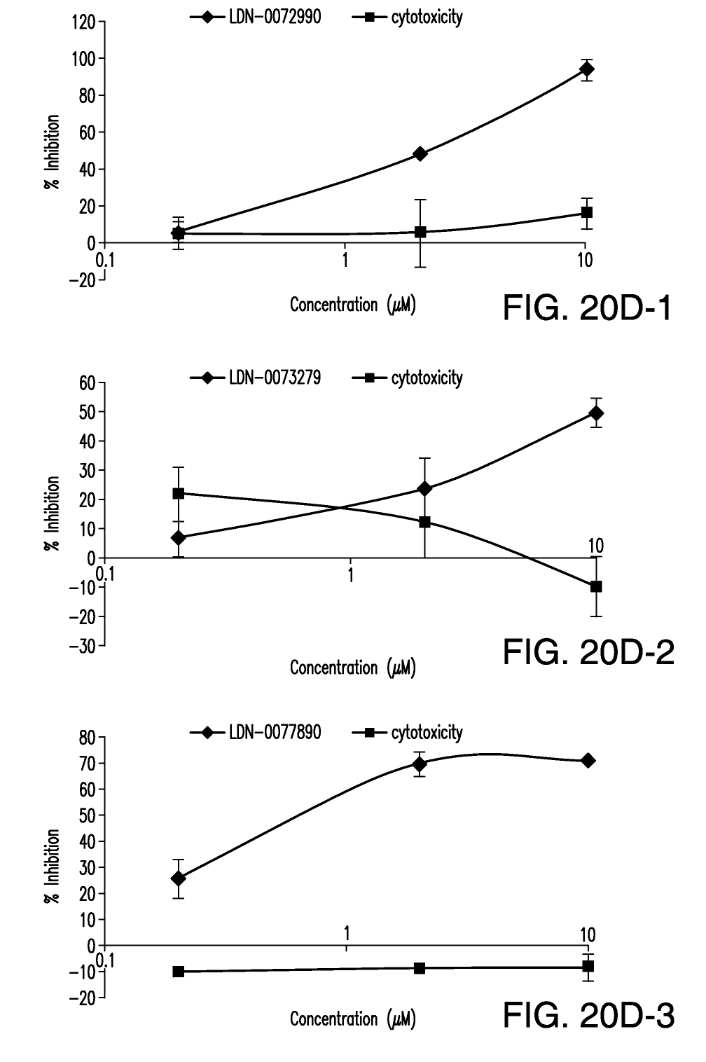

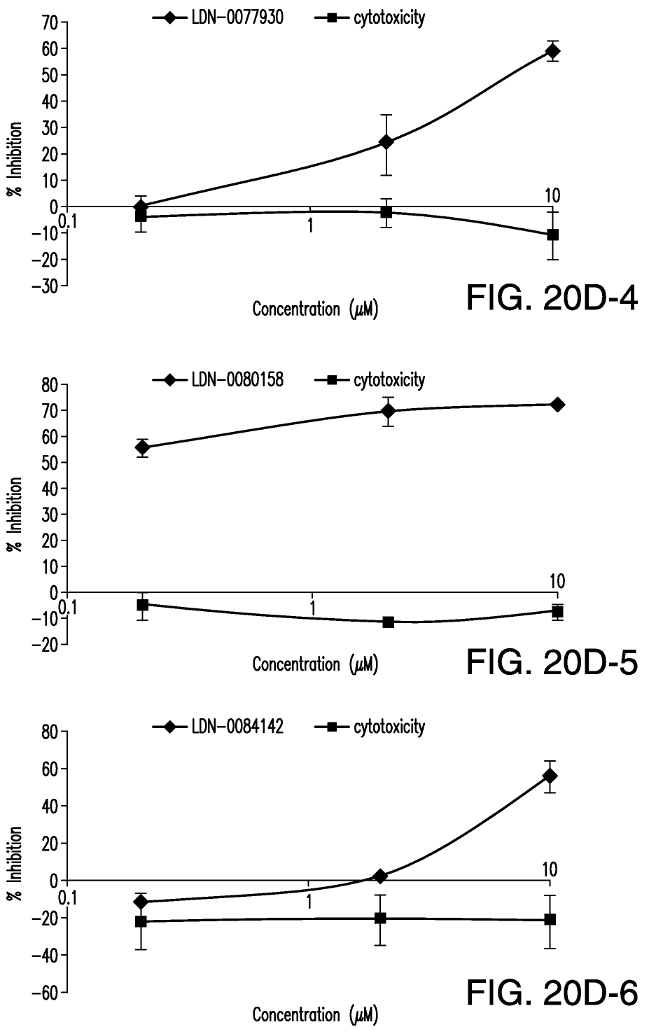

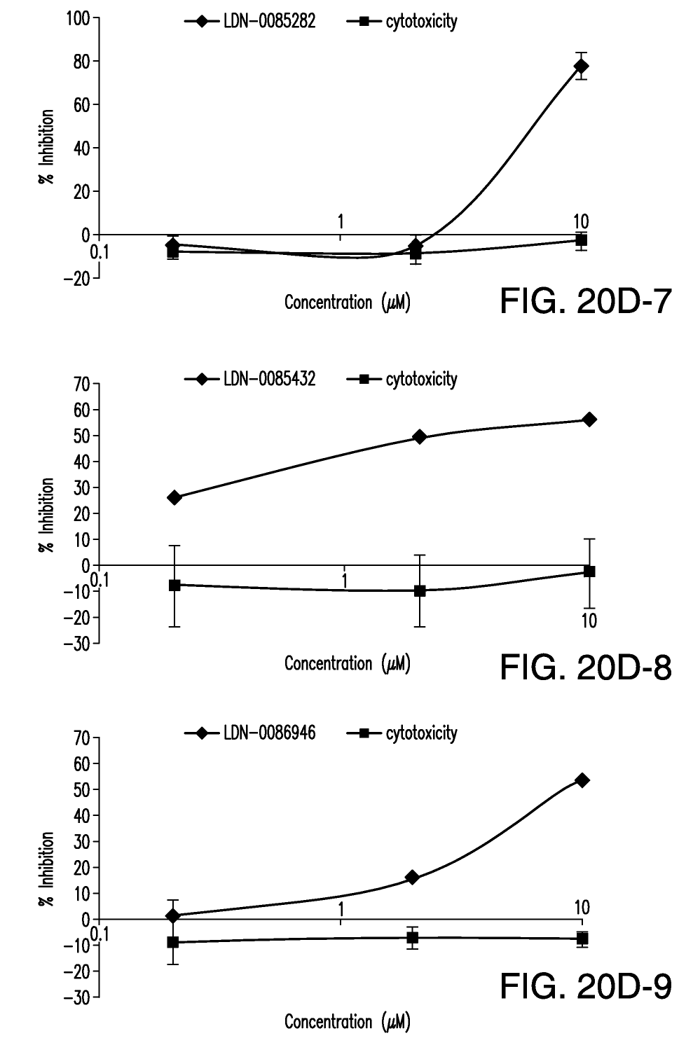

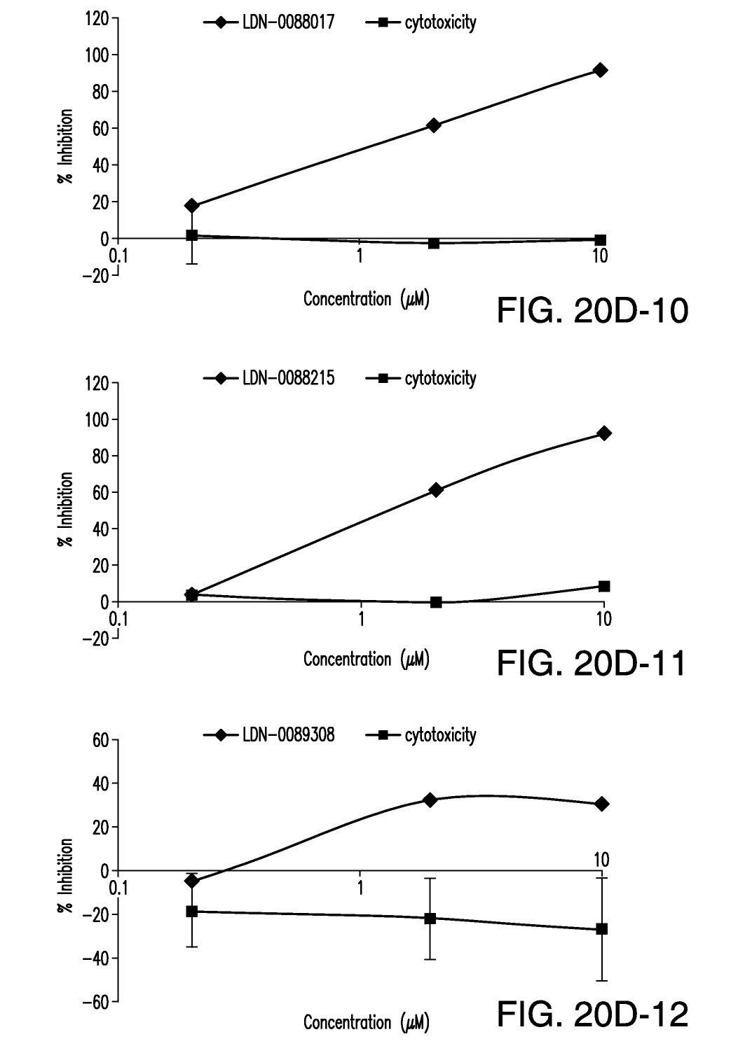

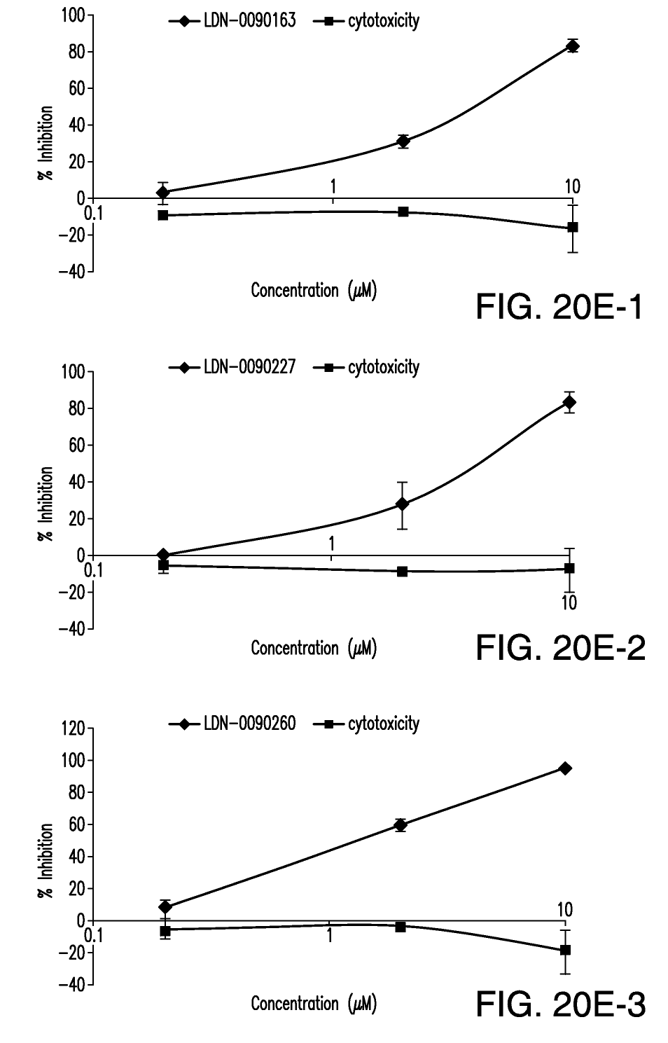

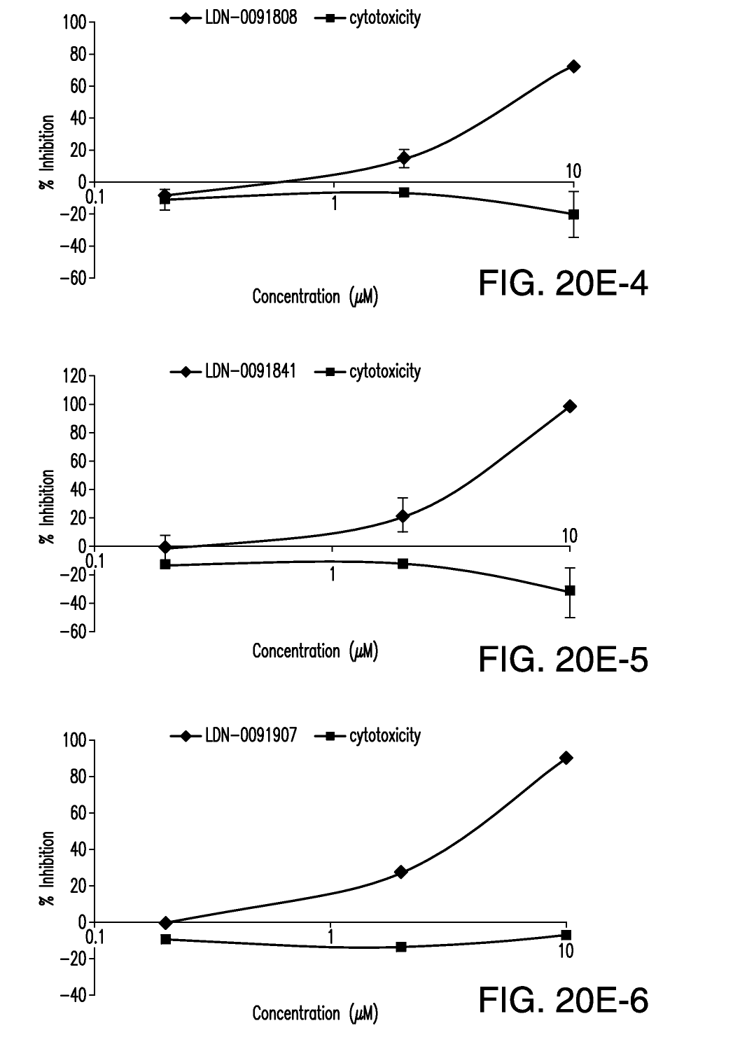

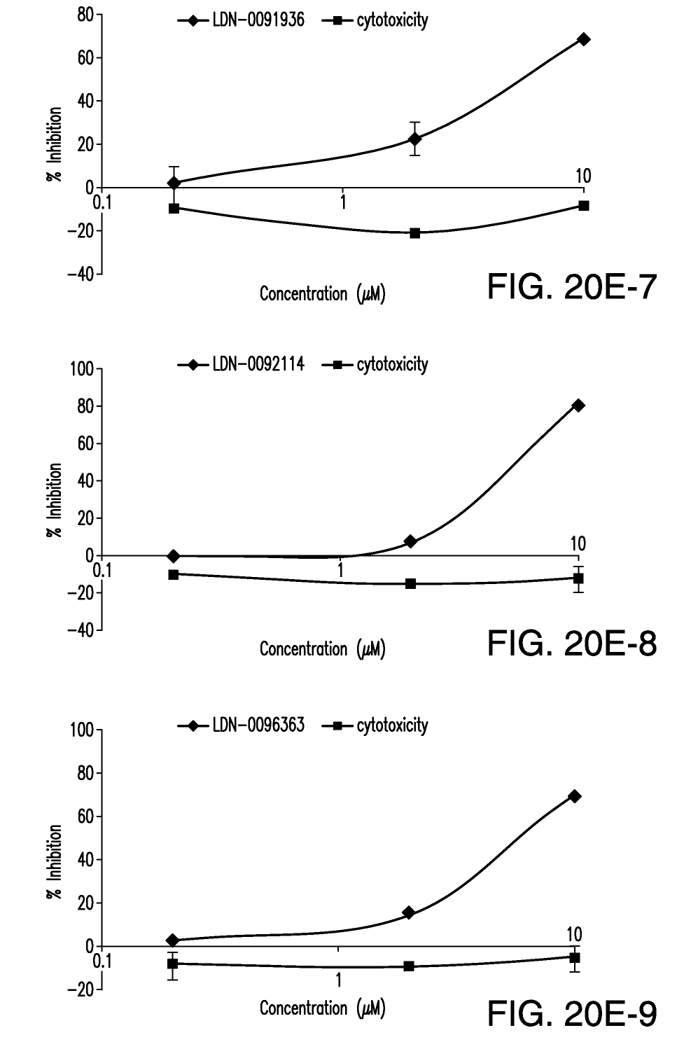

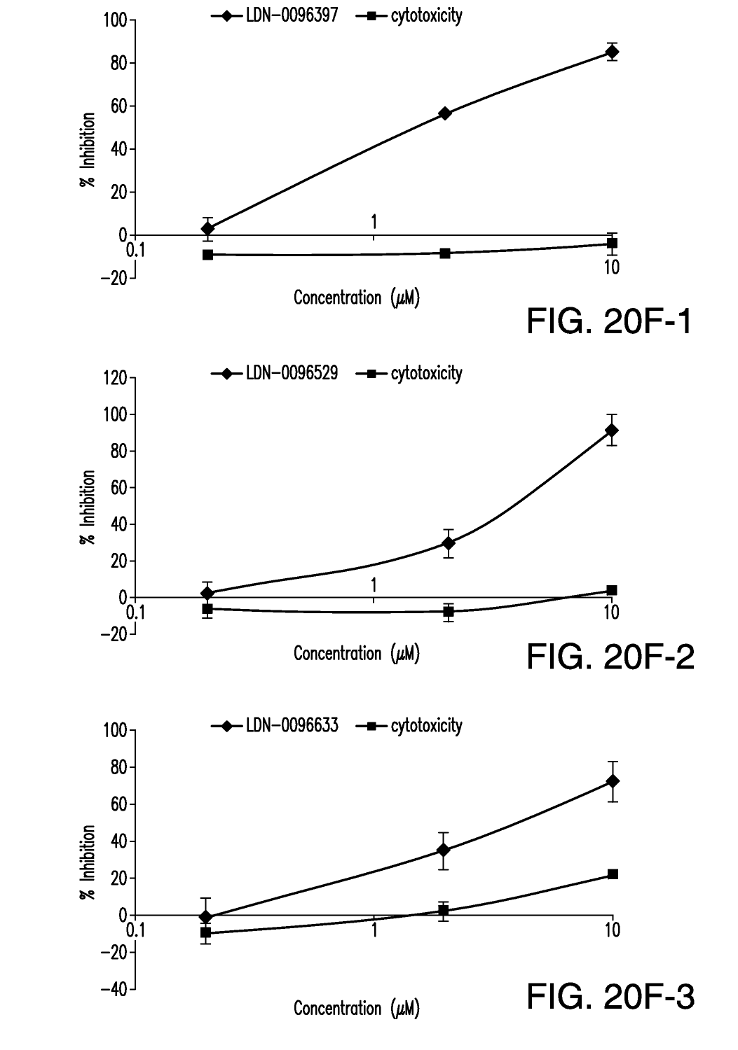

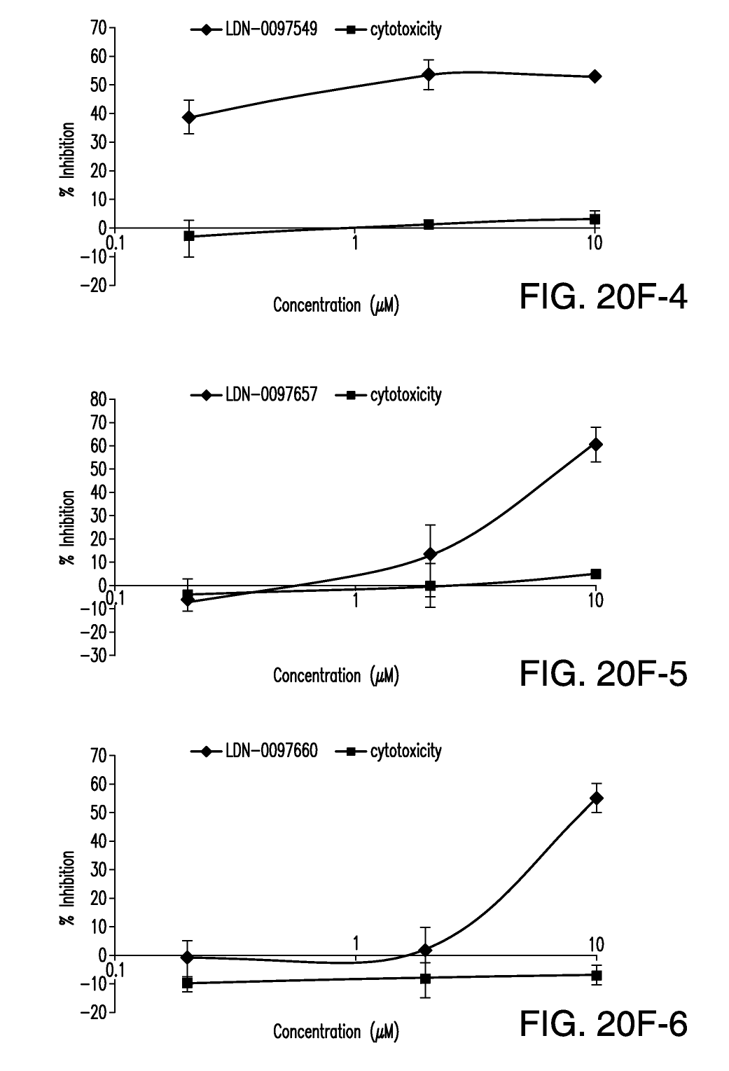

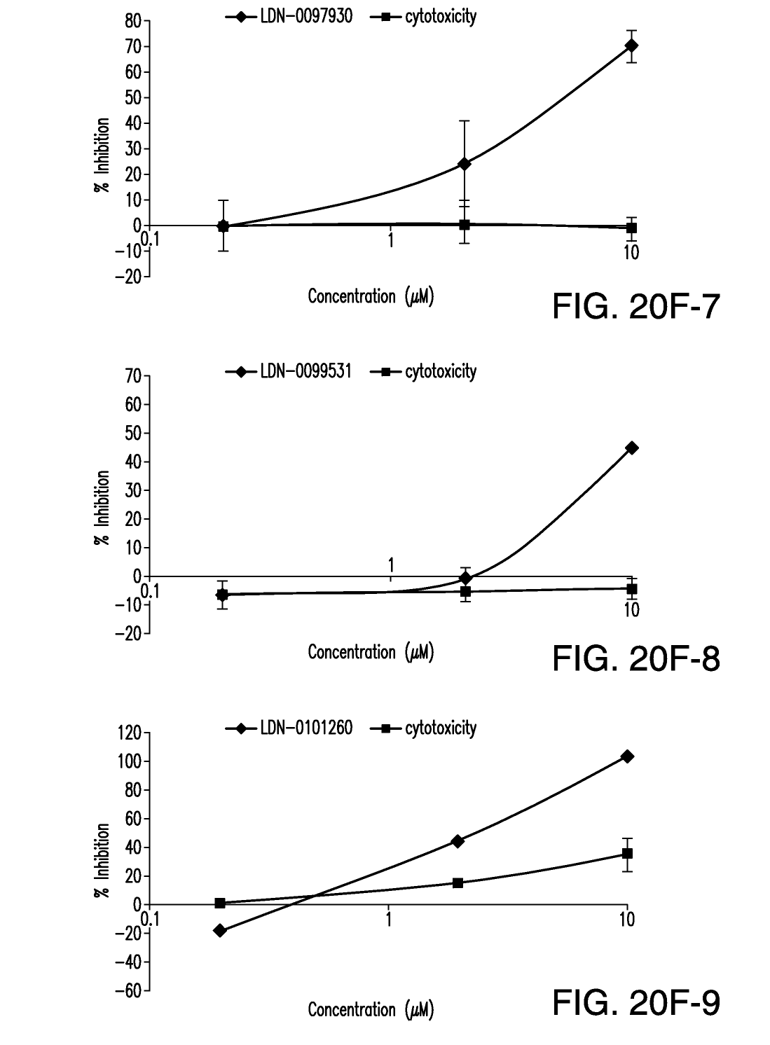

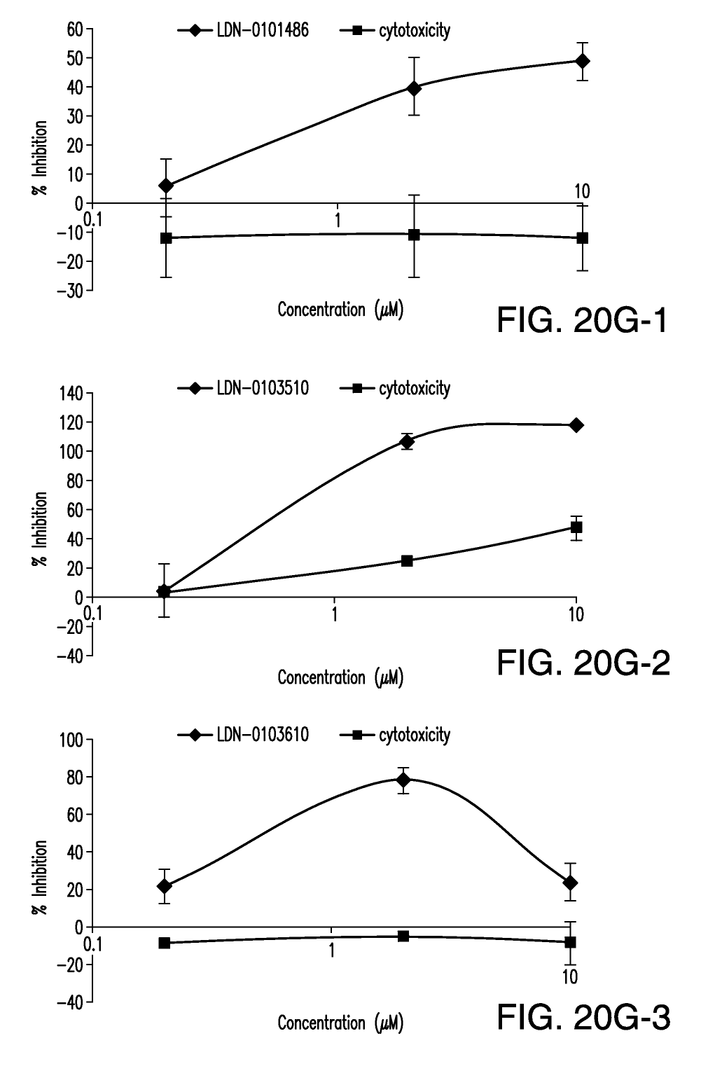

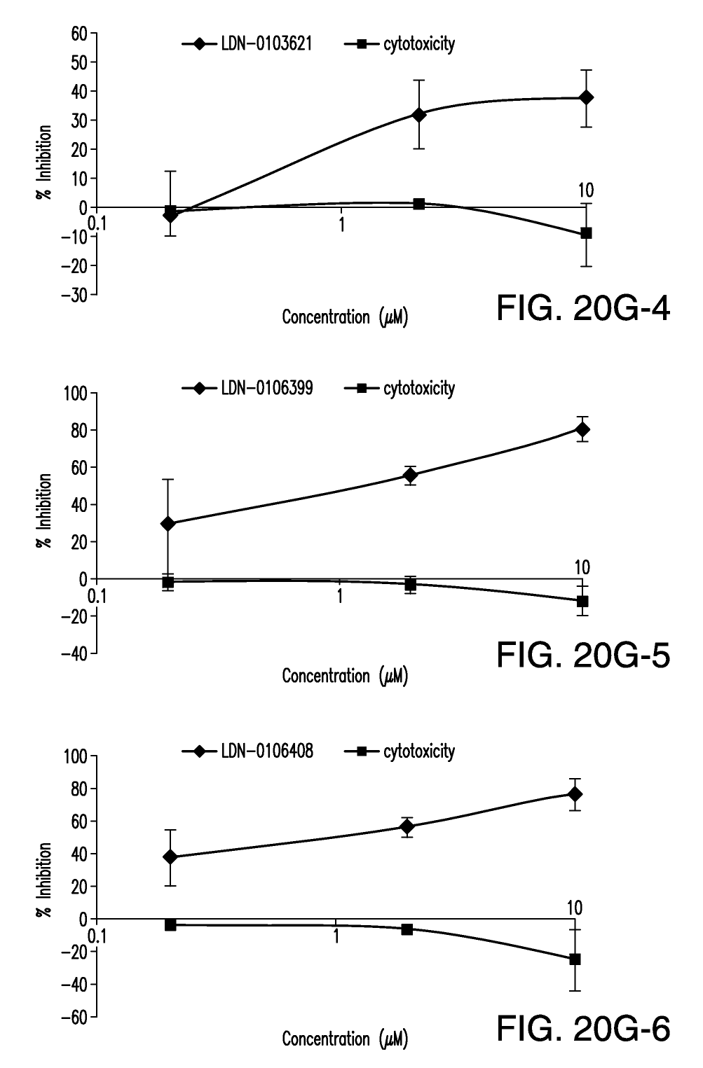

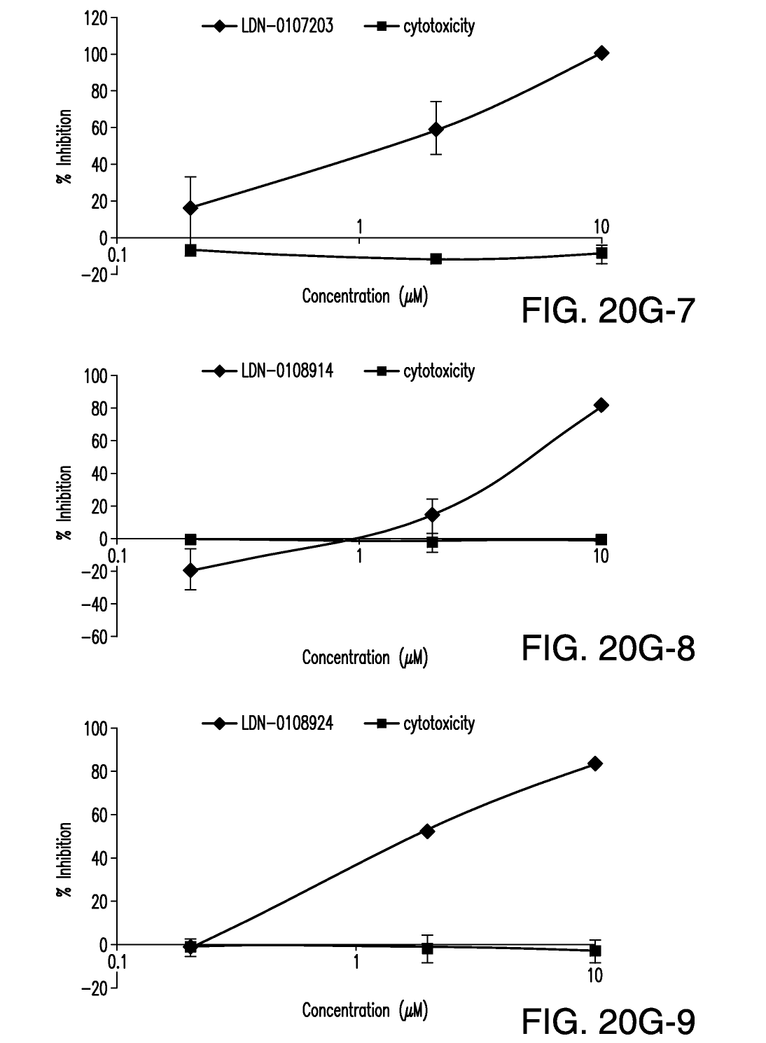

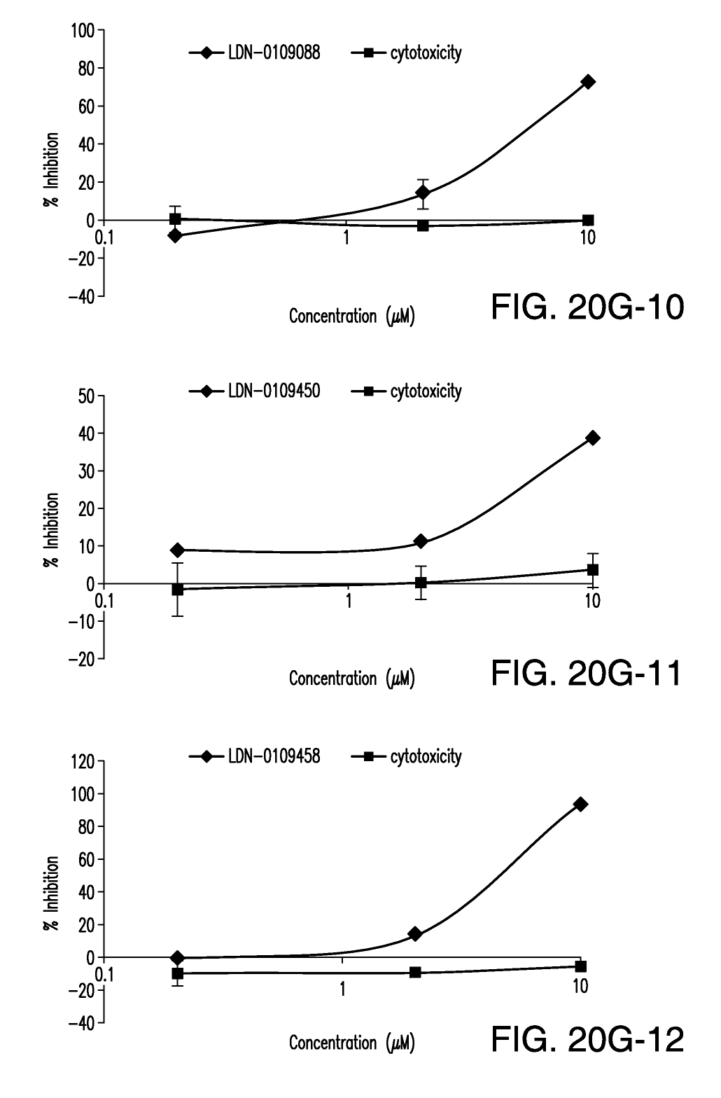

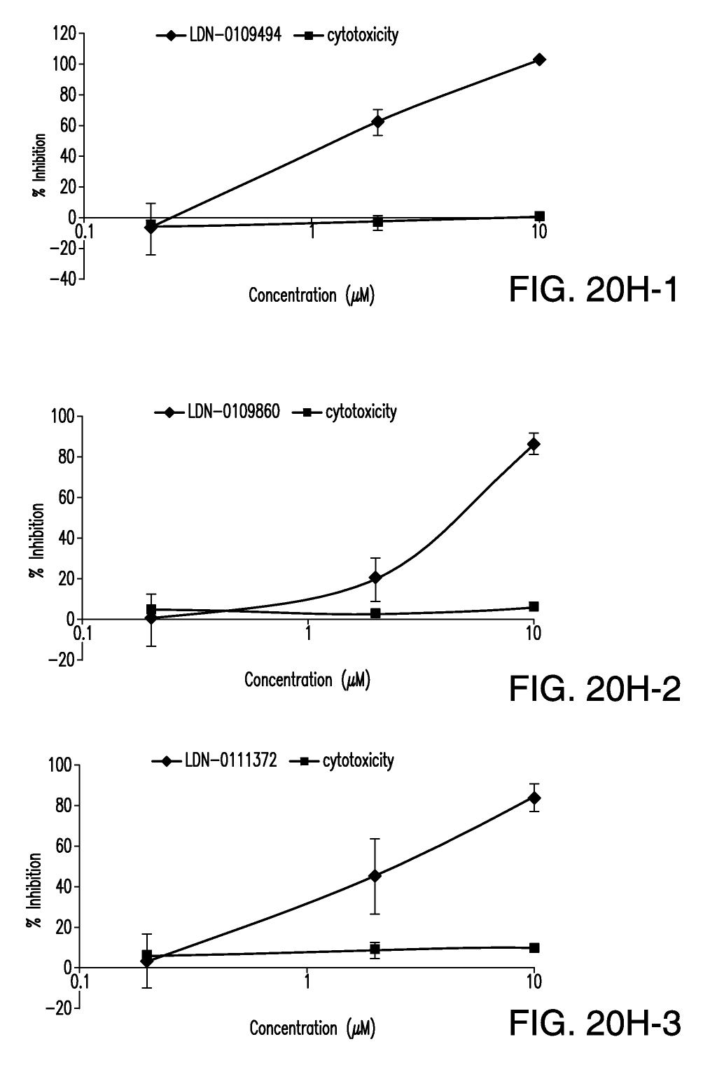

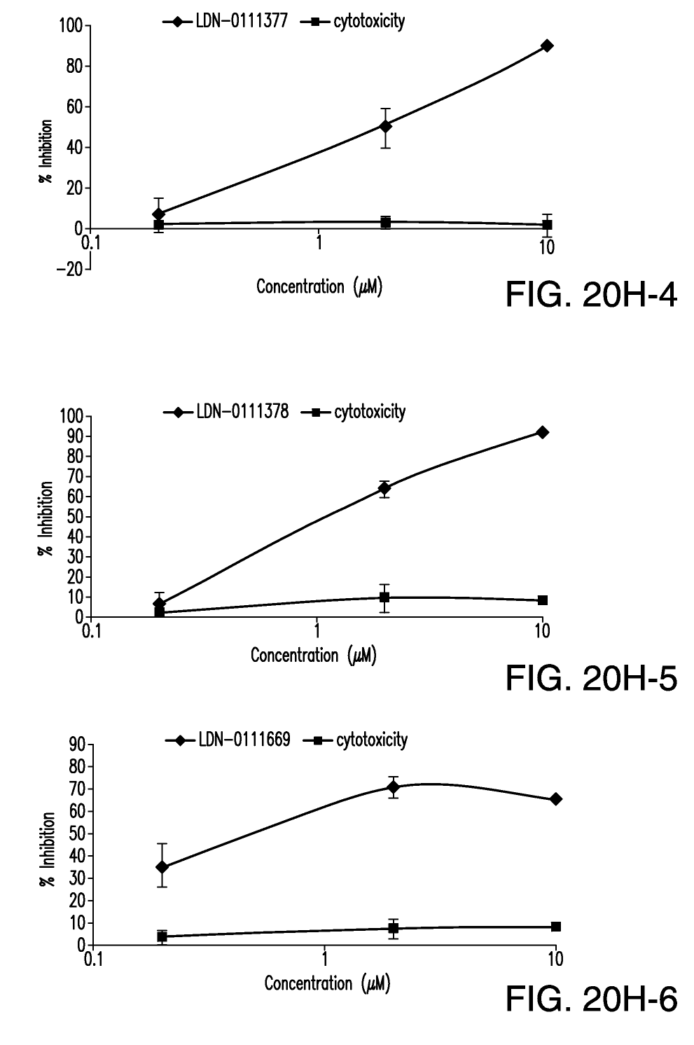

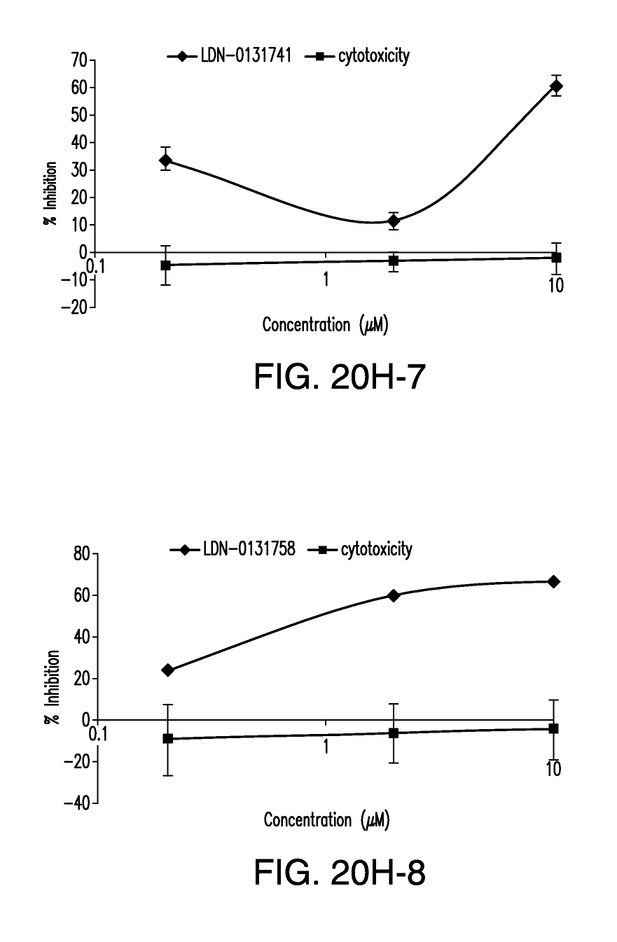

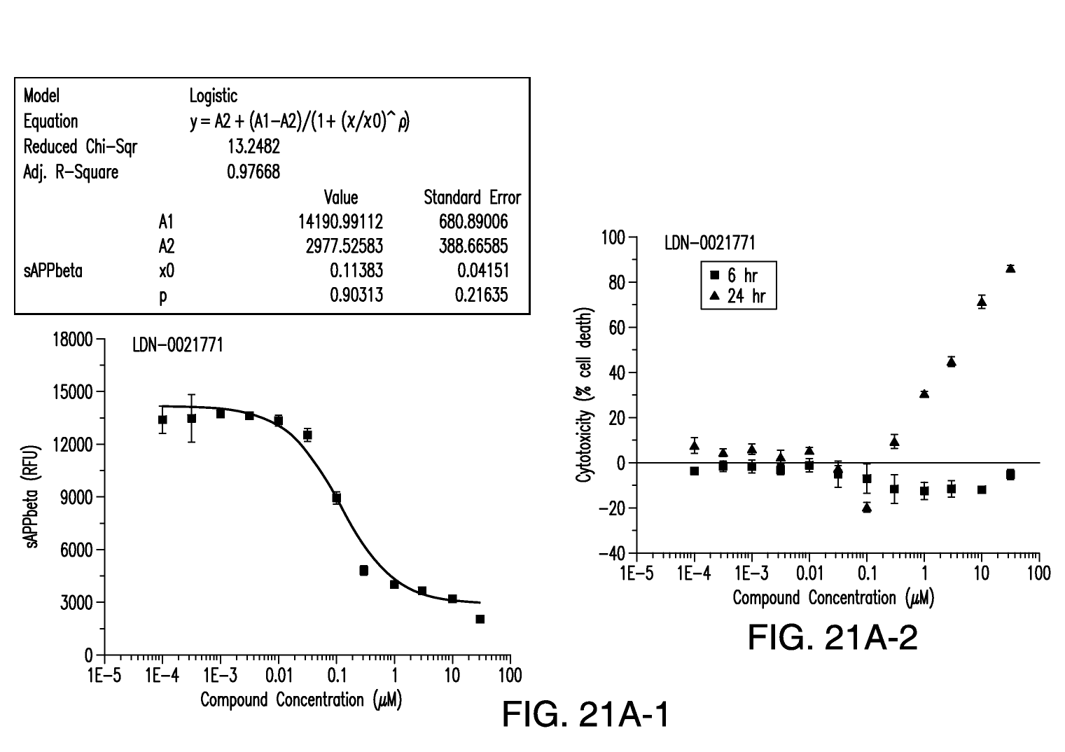

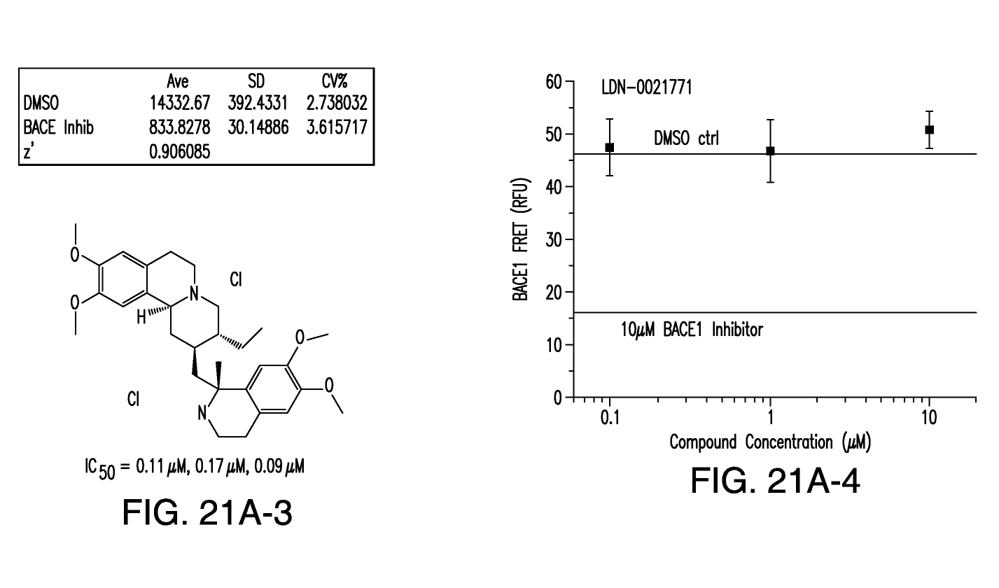

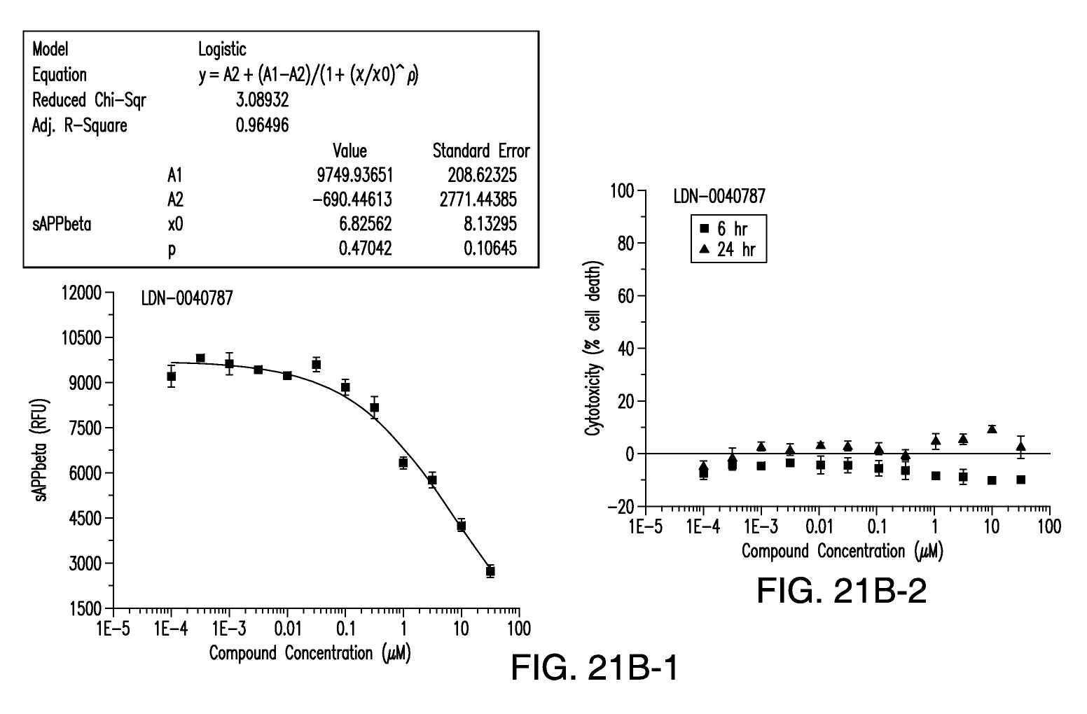

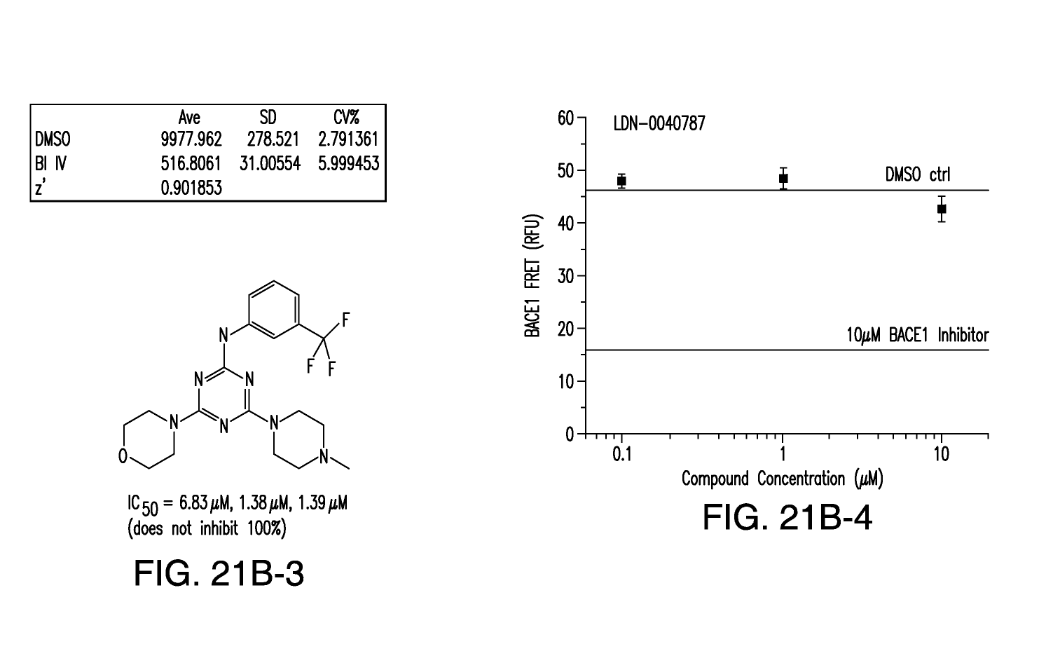

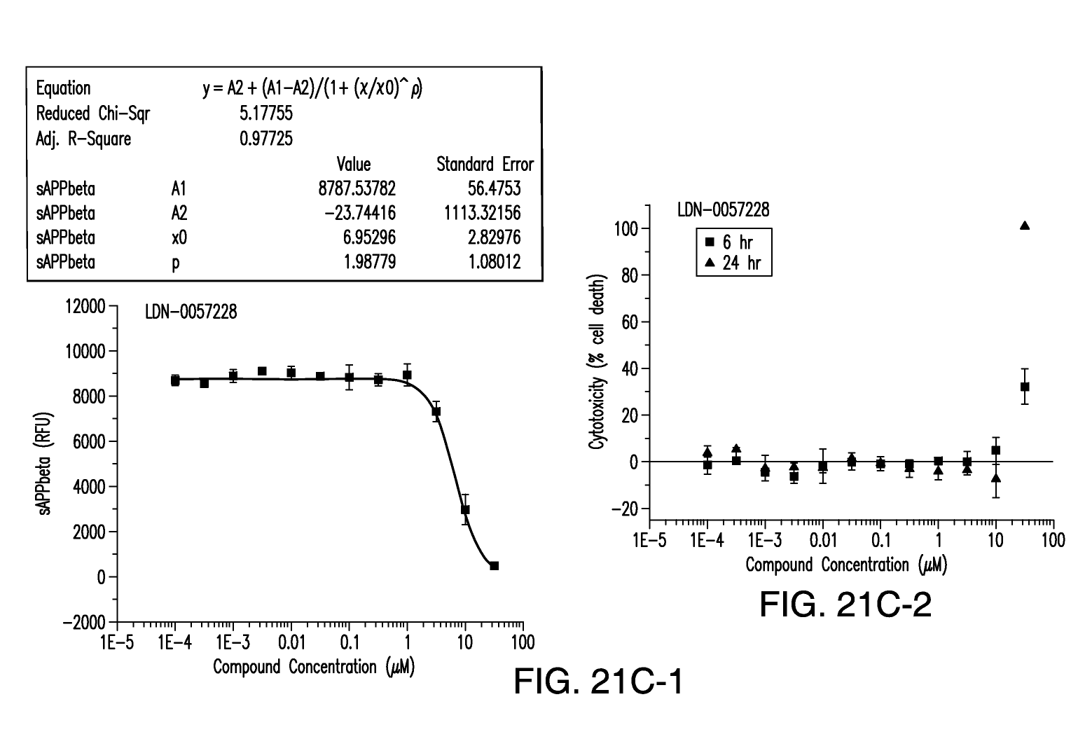

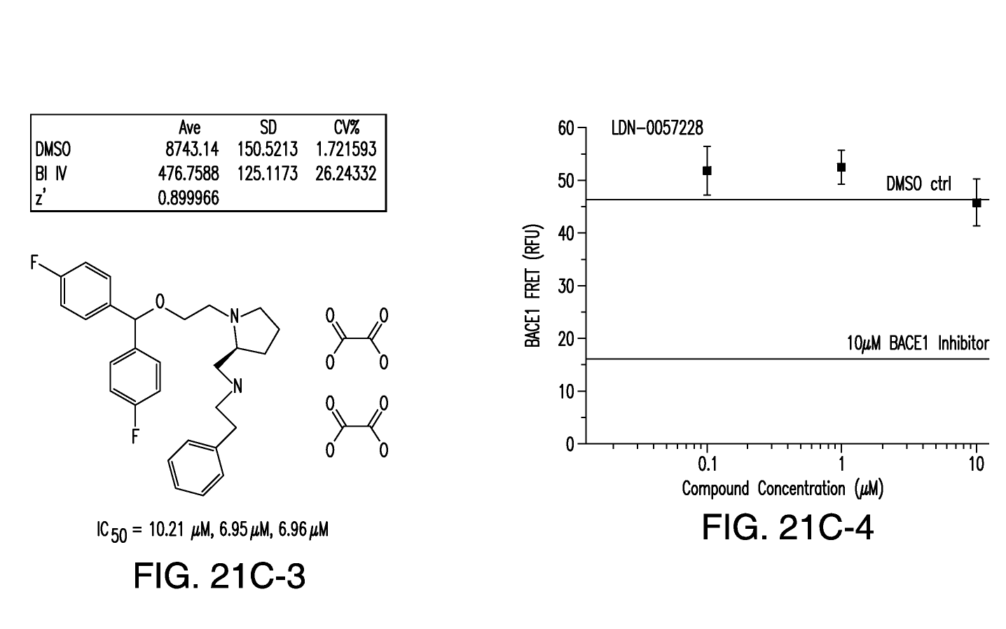

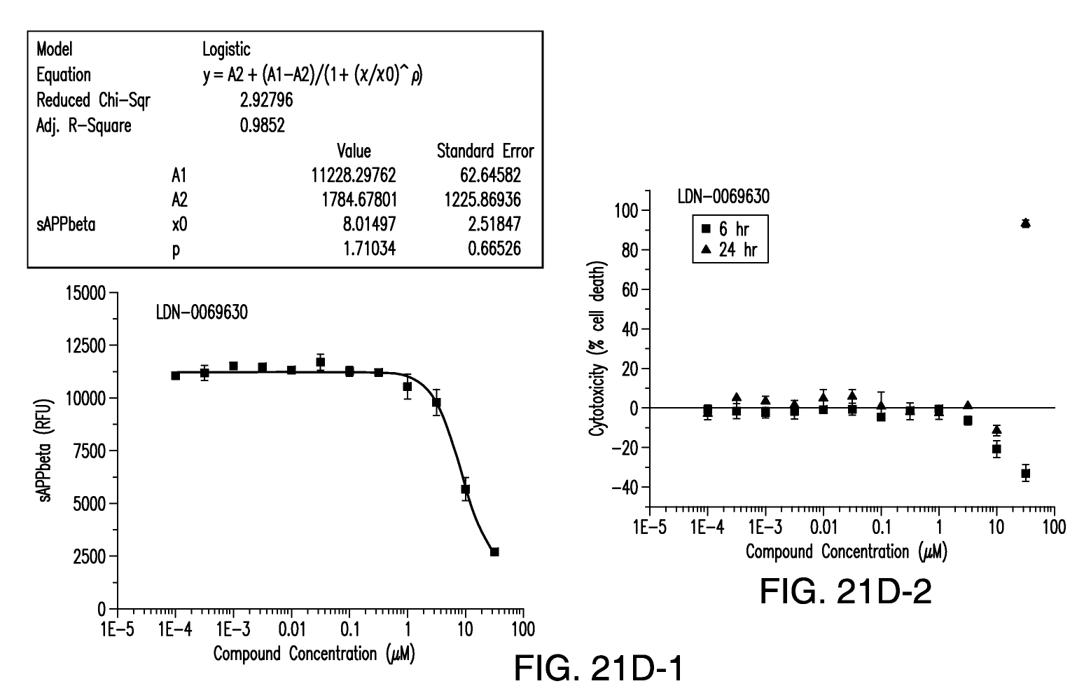

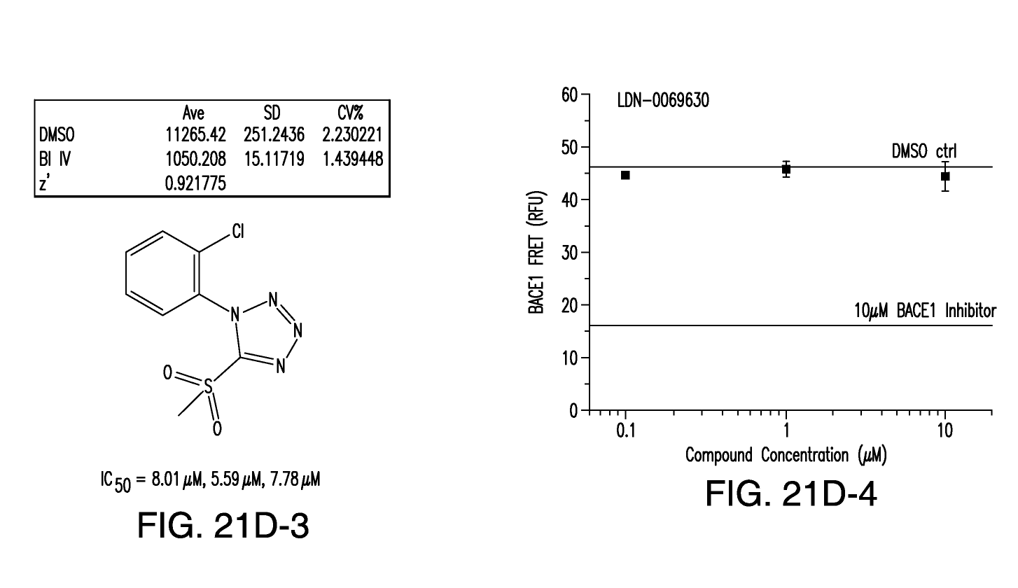

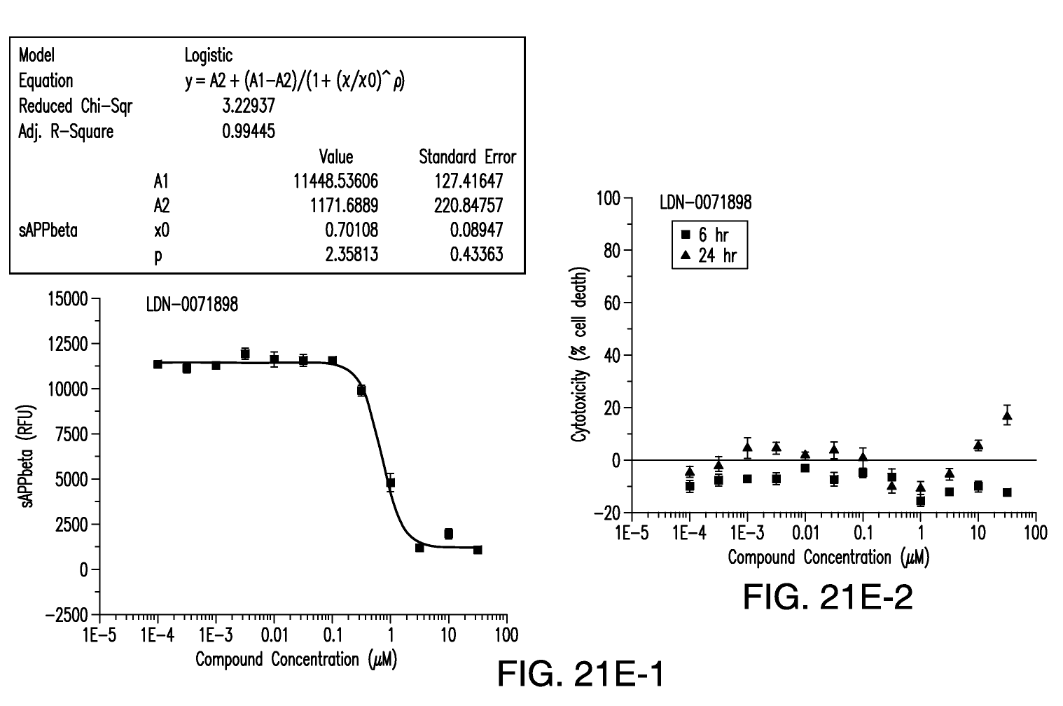

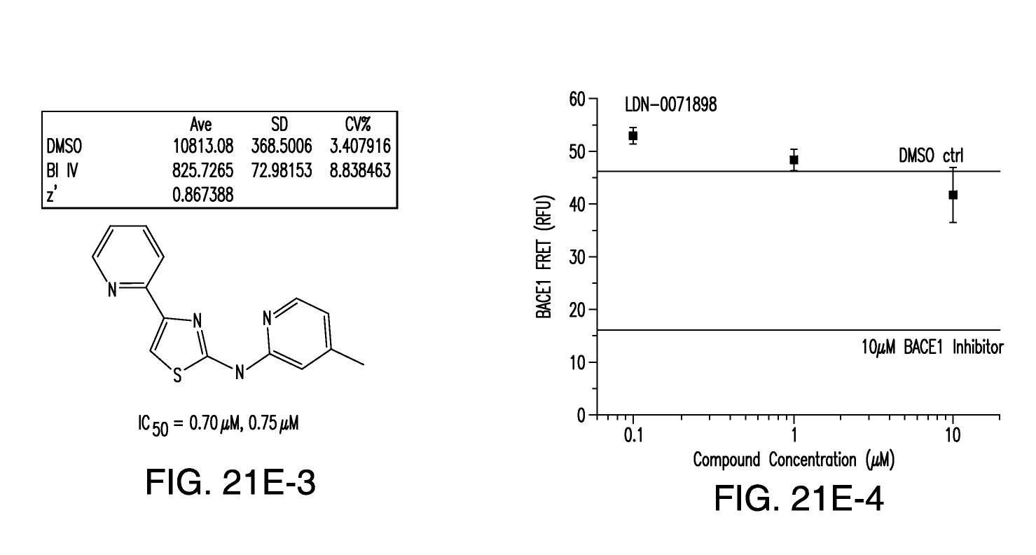

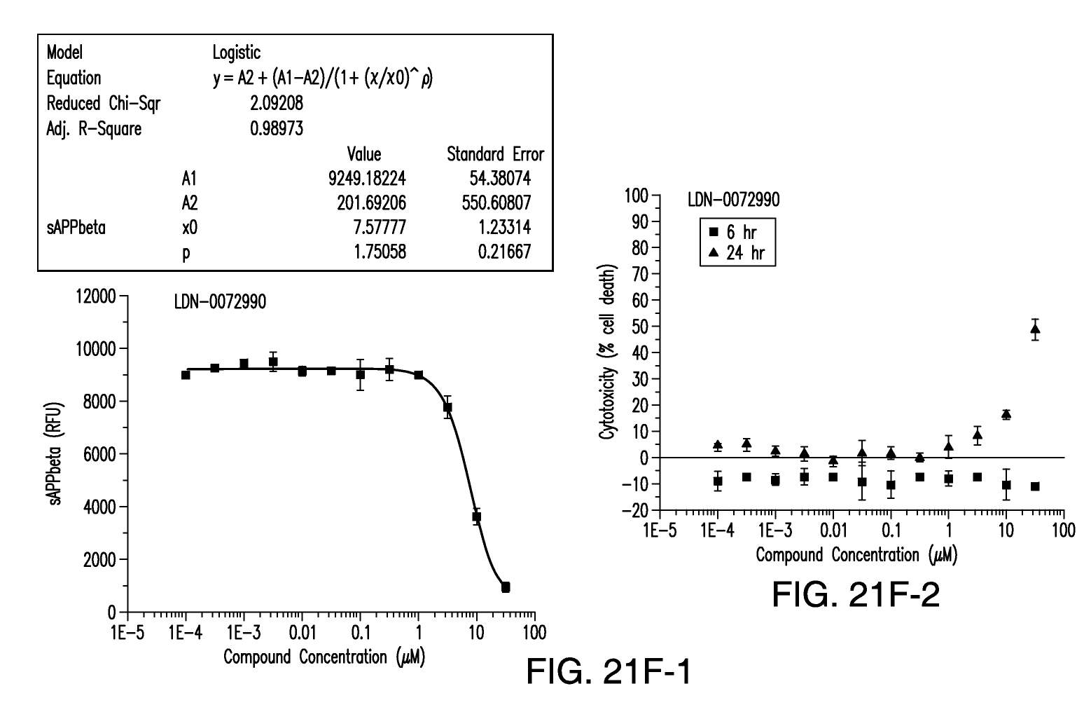

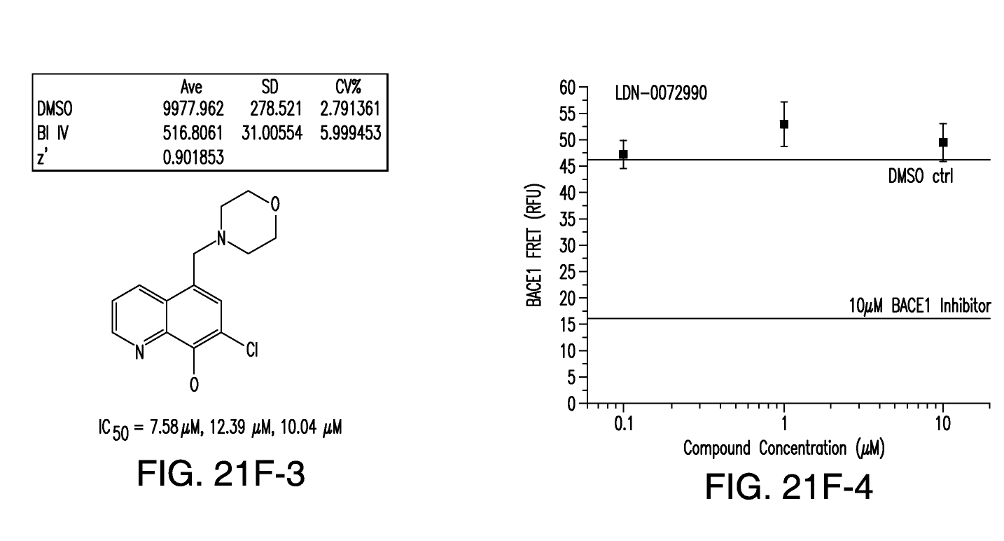

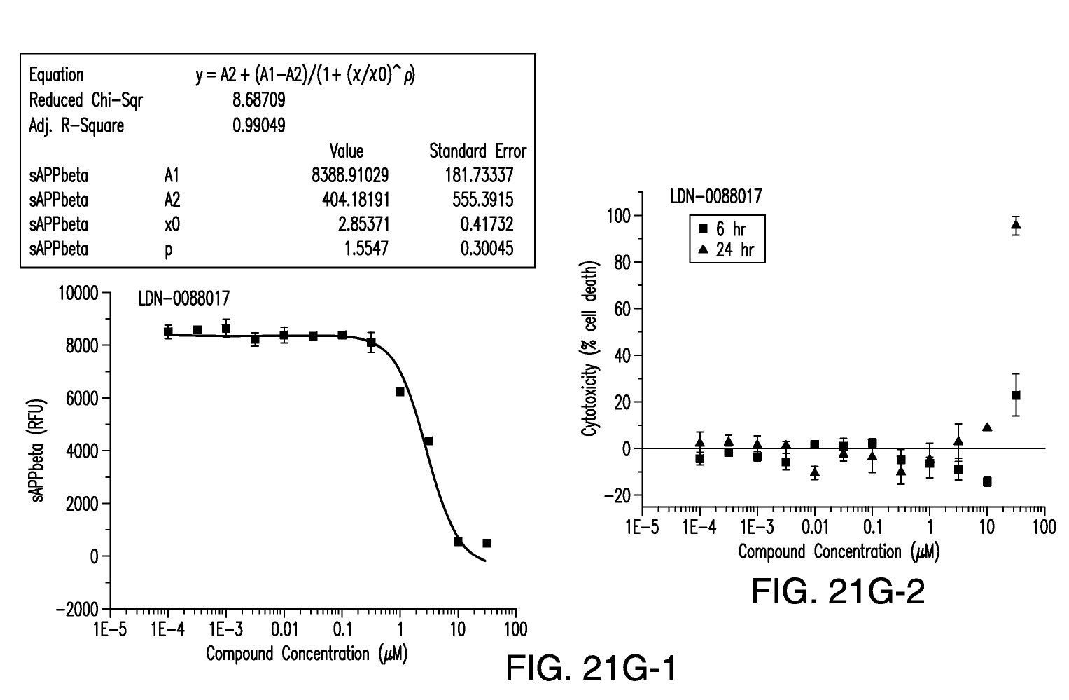

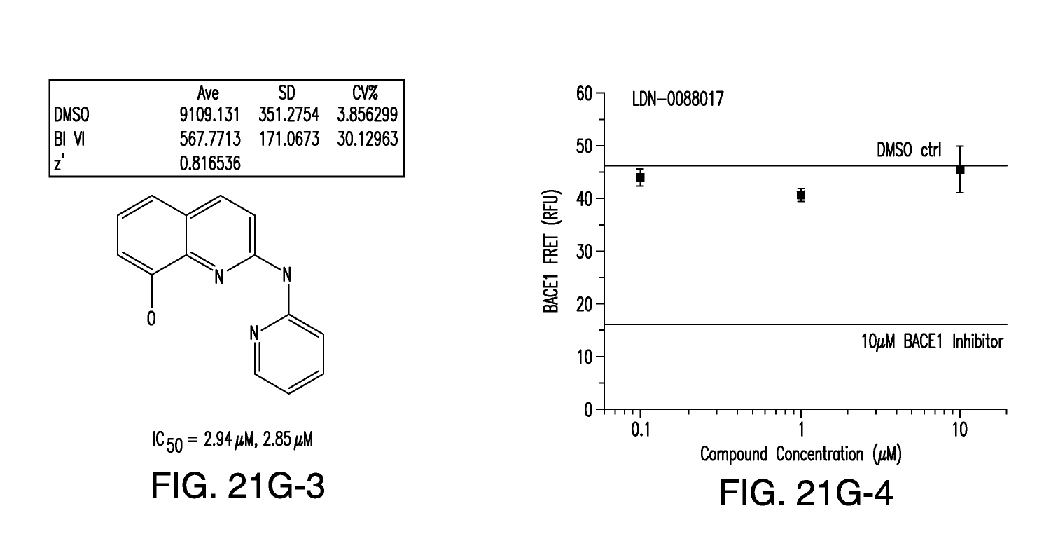

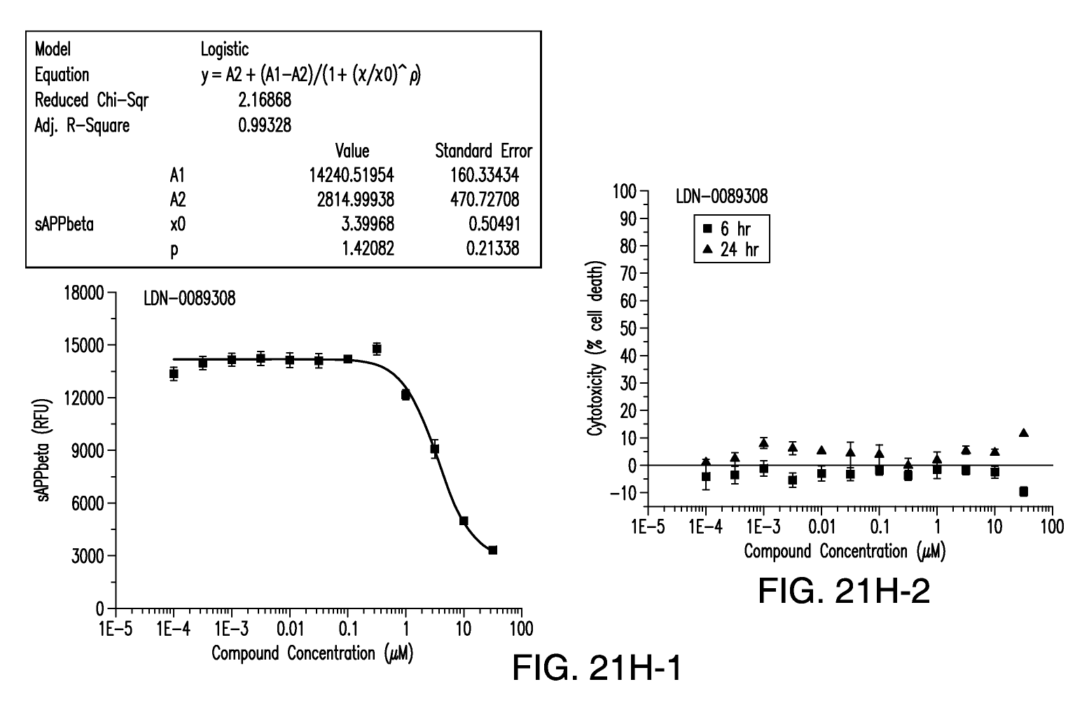

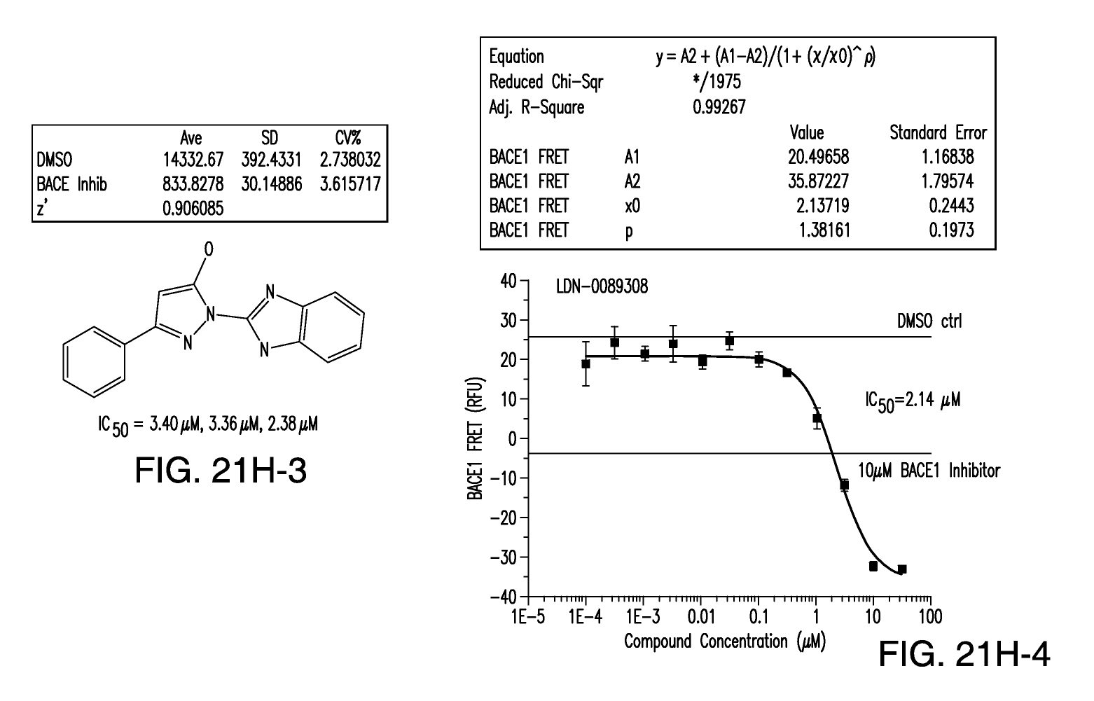

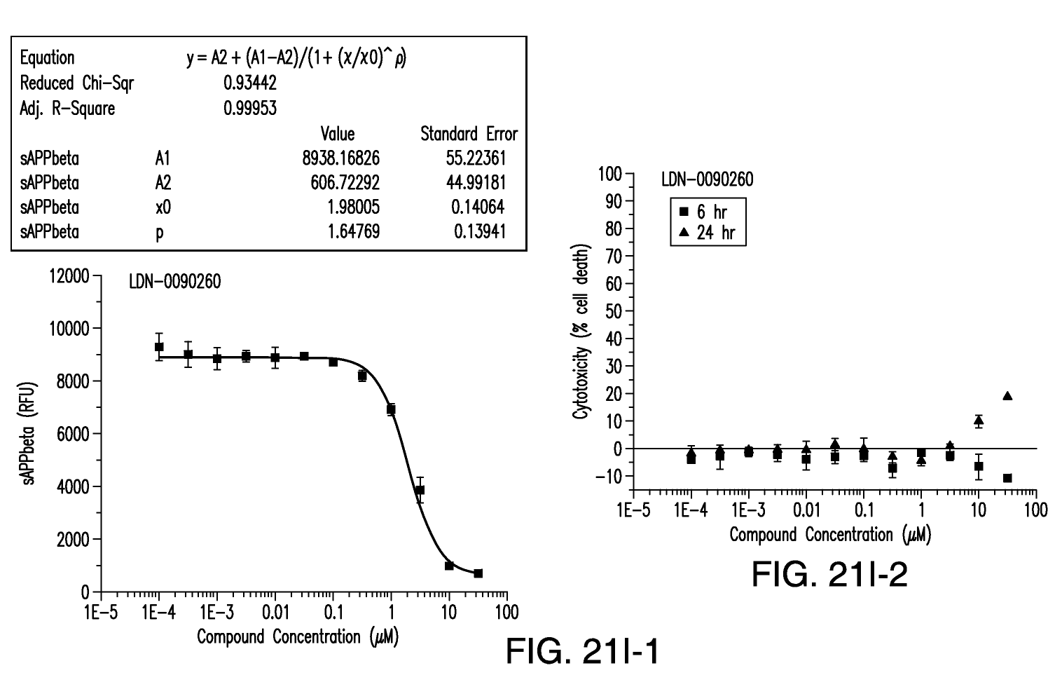

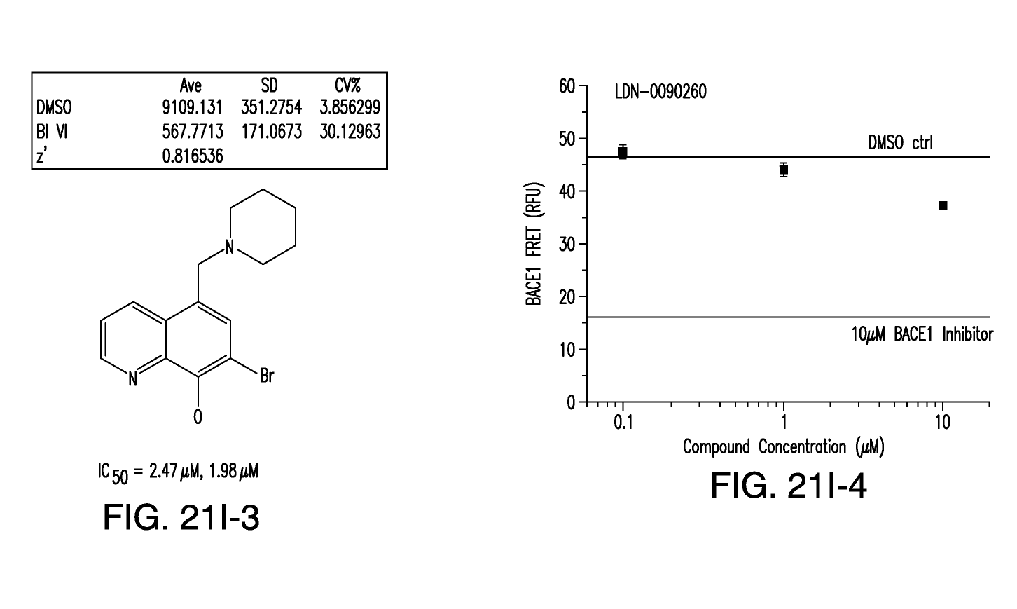

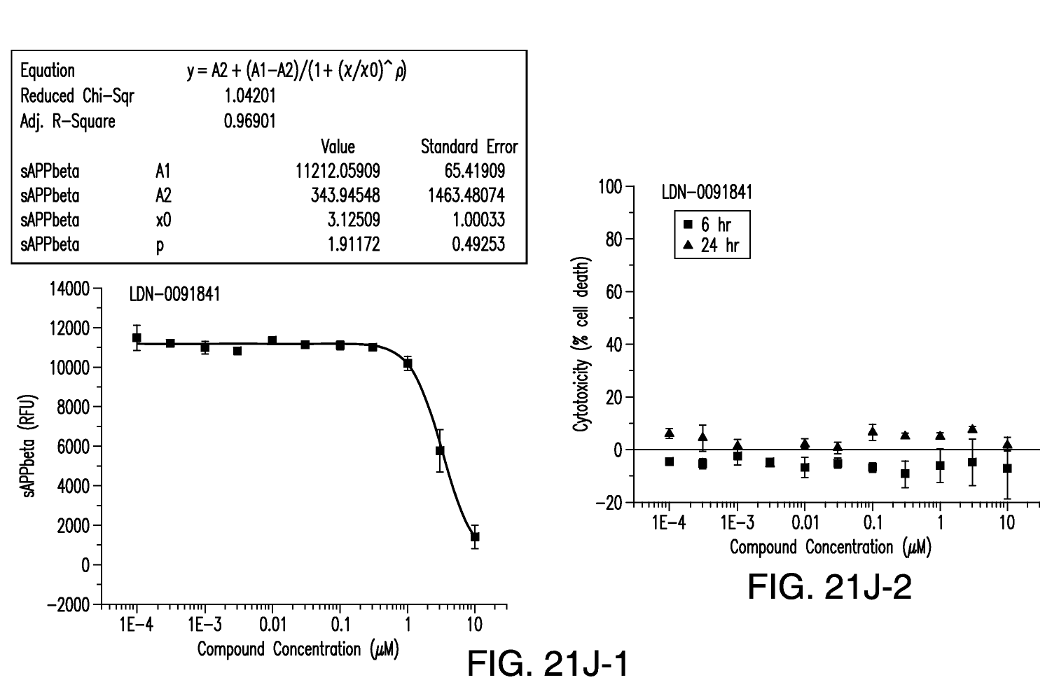

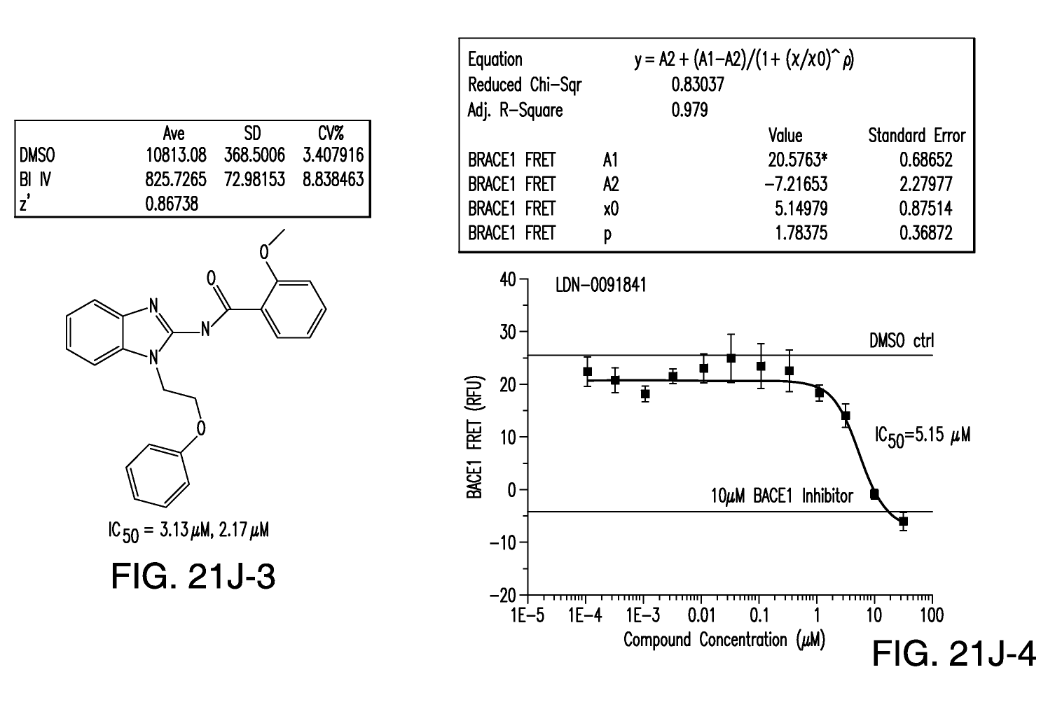

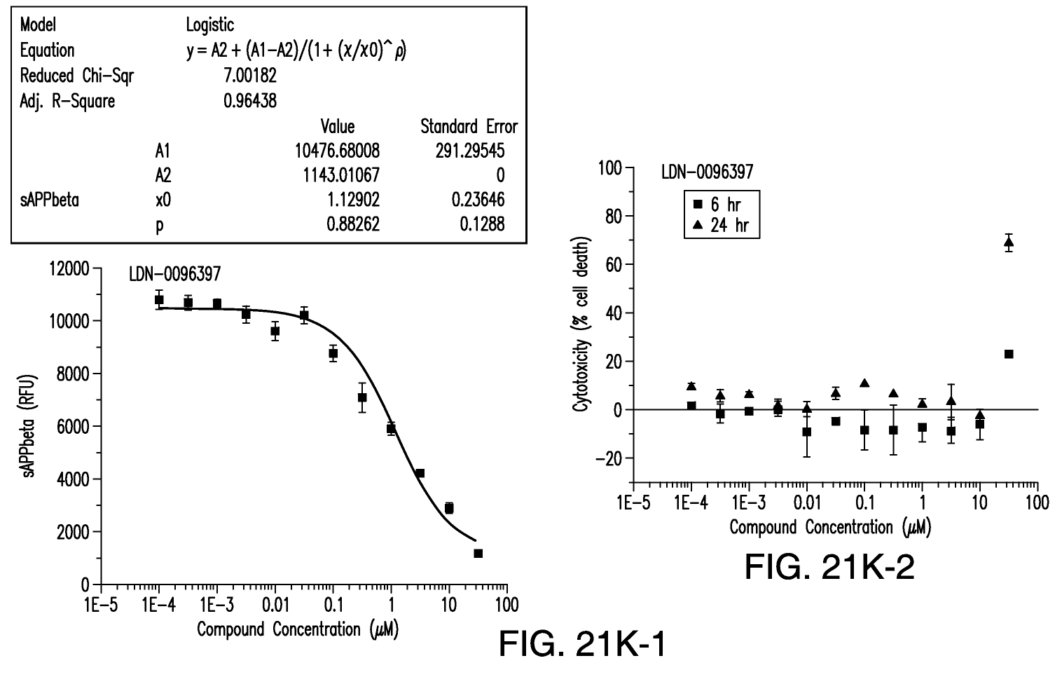

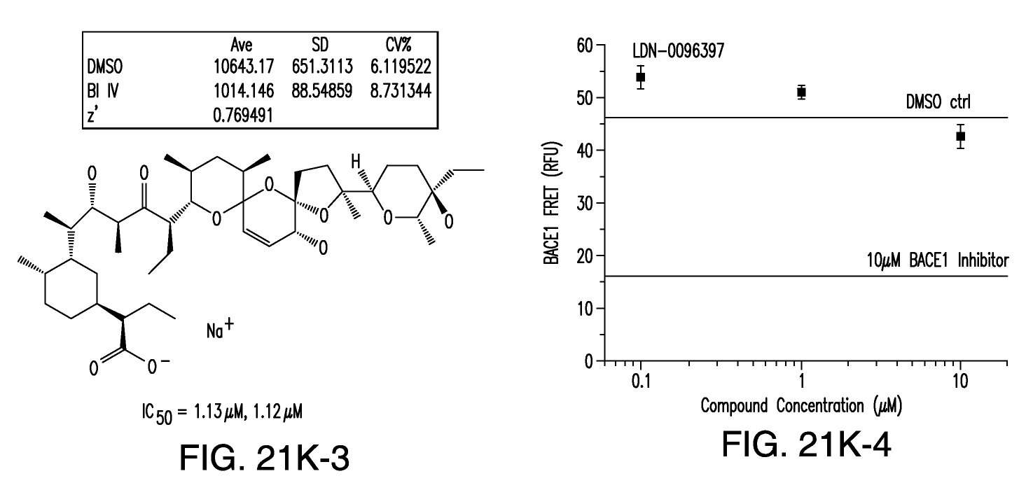

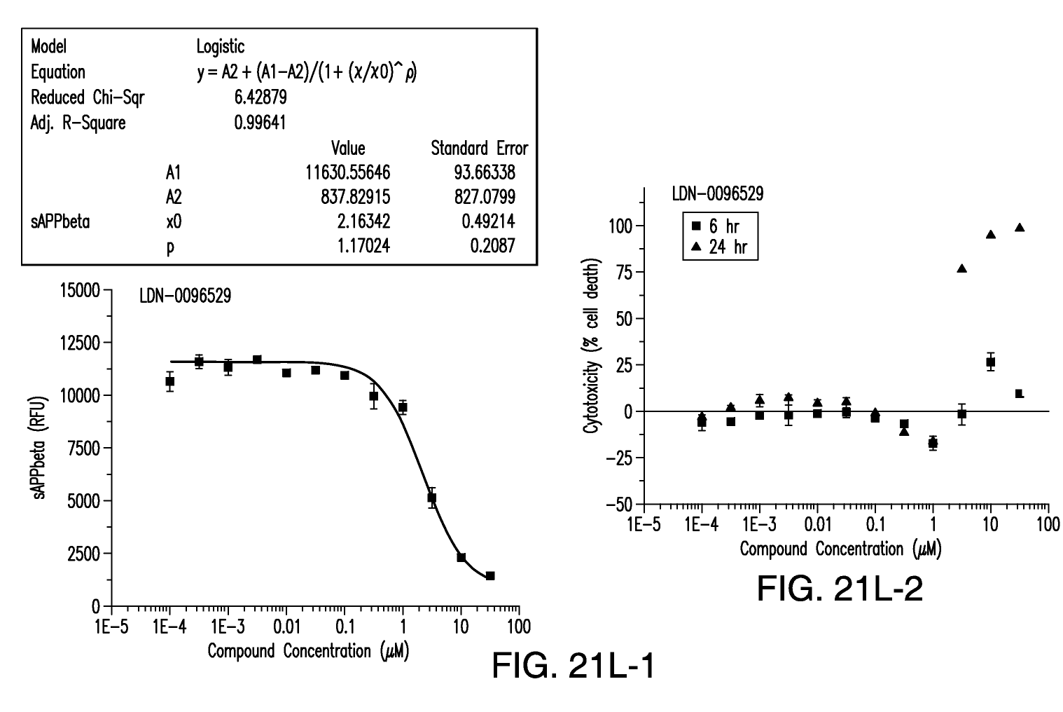

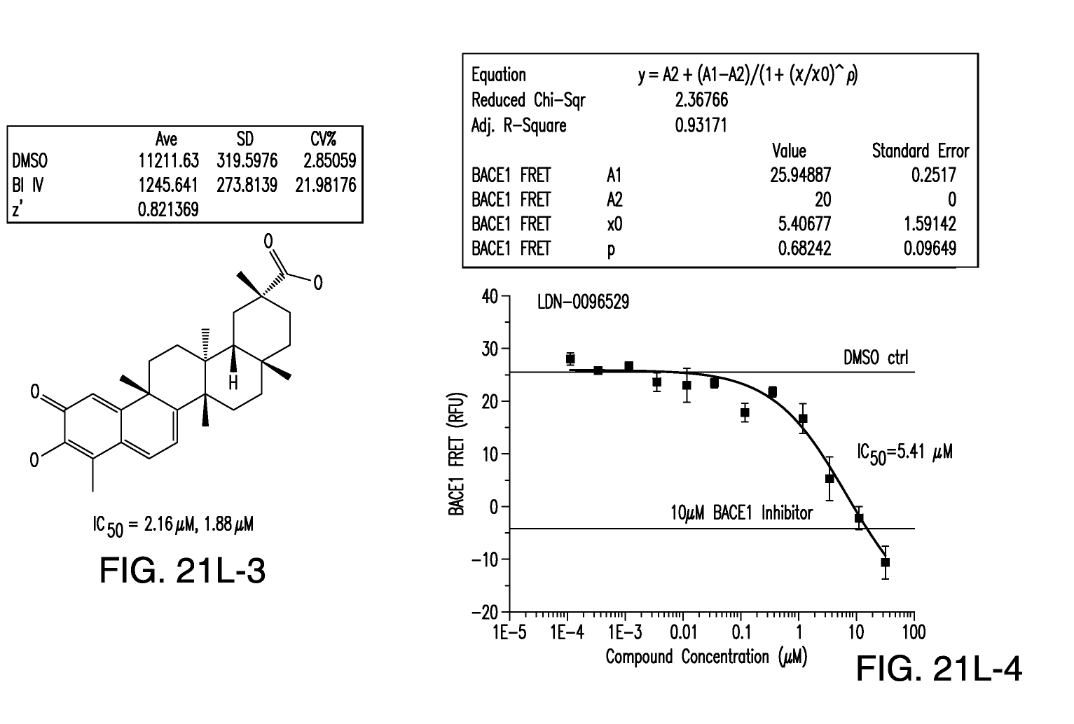

In other embodiments, the invention provides for compounds depicted in FIG. 19.

### 5.3 Methods of Treatment

In accordance with the invention, there are provided methods of using the compounds of Formulas I-VII, and/or the compounds depicted in FIG. 19, which inhibit BACE1 activity and/or inhibit the formation of APP metabolites sAPPβ and/or Aβ to exert beneficial effects. As such, these compounds may be used to treat neurodegenerative diseases, such as Alzheimer's disease.

### 5.3.1 Treatment of Neurodegenerative Diseases

The present invention provides for methods of treating a neurodegenerative disease in a subject in need of such treatment comprising administering, to the subject, a therapeutically effective amount of at least one compound of Formulas I-VII, and/or at least one compound depicted in FIG. 19. Non-limiting examples of neurodegenerative diseases include Alzheimer's disease, lewy body dementia, inclusion body myositis, and cerebral amyloid angiopathy.

In particular embodiments, the present invention provides for methods of treating diseases related to metabolism of APP by BACE1 in a subject in need of such treatment by administration of a therapeutic formulation which comprises an effective amount of at least one compound of Formulas I-VII, and/or at least one compound depicted in FIG. 19. In particular embodiments, the formulation may be administered to a subject in need of such treatment in an amount effective to inhibit BACE1 activity and/or reduce the production of sAPPβ and/or Aβ. Where the formulation is to be administered to a subject in vivo, the formulation may be administered systemically (e.g. by intravenous injection, oral administration, inhalation, etc.), intraventricularly, intrathecally, or by any other means known in the art. The amount of the formulation to be administered may be determined using methods known in the art, for example, by performing dose response studies in one or more model system, followed by approved clinical testing in humans.

In one embodiment, the subject or patient has been diagnosed with, or has been identified as having an increased risk of developing a neurodegenerative disease, such as Alzheimer's Disease.

In other non-limiting embodiments, the present invention provides for methods of reducing, in a subject, the risk of neural damage related to increased levels of Aβ and/or sAPPβ comprising administering, to the subject, an effective amount of a composition according to the invention. An effective amount may be a local concentration or, in a pharmaceutical composition, an amount that, when administered to a subject, results in a therapeutic benefit.

According to the invention, an effective amount is an amount of at least one compound of Formulas I-VII, and/or at least one compound depicted in FIG. 19, which reduces one or more clinical symptom of one or more of the aforementioned diseases and/or reduces neural damage related to metabolism of APP by BACE1. In one example, an effective amount is an amount of at least one compound of Formulas I-VII, and/or at least one compound depicted in FIG. 19, that reduces the production of sAPPβ or Aβ generated by the metabolism of APP by BACEI.

In one non-limiting embodiment, the effective amount of at least one compound of Formulas I-VII, and/or at least one compound depicted in FIG. 19, may be determined via an in vitro assay, for example, as described in International Patent Application No. PCT/US2007/015938 (Publication No. WO 2008/008463), which is incorporated in its entirety herein for all purposes, wherein the effective amount may be correlated with the compound's ability to reduce the level of sAPPβ. By way of example, and not of limitation, such an assay may comprise a cell-based modified ELISA assay for measuring sAPPβ, the secreted ectodomain of β-amyloid precursor protein (APP) following β-secretase (BACE1) cleavage. Such an in vitro assay may be used to identify compounds of Formulas I-VII, and/or compounds depicted in FIG. 19, that interfere with the first step of sAPPβ production.

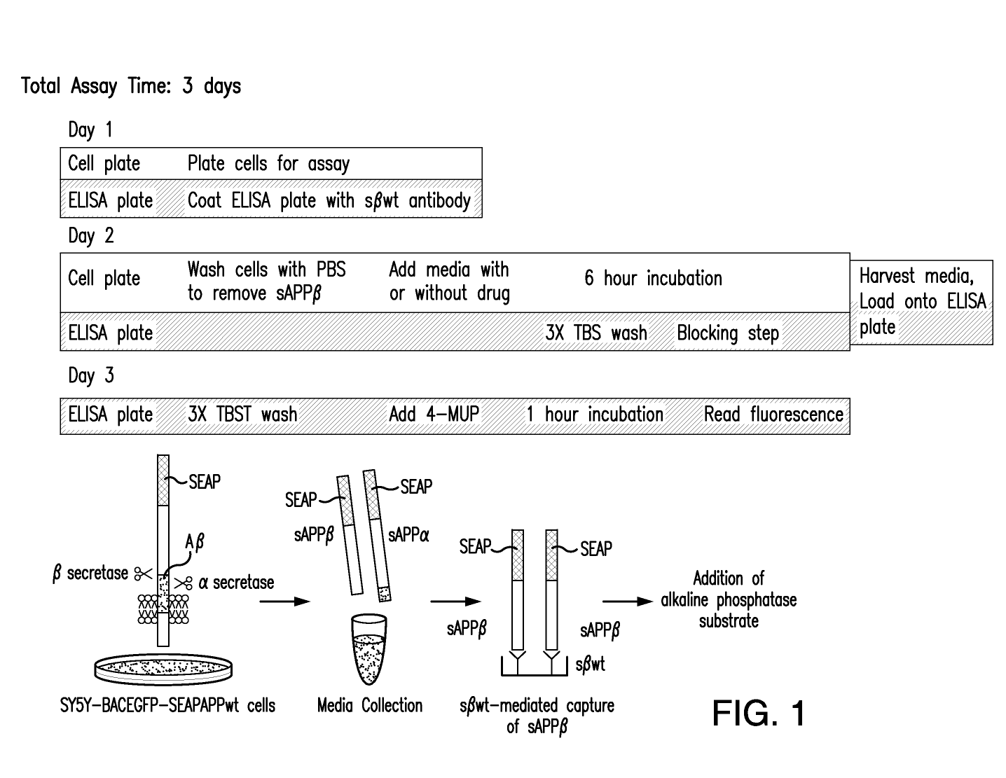

As described in the Examples below, and depicted in FIG. 1, an sAPPβ ELISA assay may comprise cells, for example, SY5Y cells, transfected with a BACE1 reporter construct, such as a GFP-tagged BACE1 (BACE-GFP), and a wild type APP reporter construct, such as a secreted alkaline phosphatase (SEAP)-tagged wild type APP (SEAP-APPwt). BACE1 cleavage of the reporter-tagged APP (e.g. SEAP-APPwt) may result in secretion into the media of SEAP-tagged sAPPβ, which may be collected and specifically captured using an sAPPβ cleavage site-specific antibody (e.g. stβwt). Following washing, a substrate may be used, for example, the fluorescent alkaline phosphatase substrate 4-methylumbelliferyl phosphate (4-MUP), to detect the captured SEAP-sAPPβ.

In one non-limiting example, an effective amount of a compound of Formulas I-VII, and/or a compound depicted in FIG. 19, may be correlated with the compound's ability to reduce the level of sAPPβ detected in the in vitro assay compared to a control cell line that was not contacted with the candidate compound, wherein a reduction of sAPPβ compared to the control cell line correlates with the compound's therapeutic efficacy.

In another non-limiting example, an effective amount of a compound of Formulas I-VII, and/or a compound depicted in FIG. 19, may be correlated with the compound's ability to reduce the level of sAPPβ detected in the in vitro assay by at least 0.1, or by at least 0.5, or by at least 1, or by at least 1.5, or by at least 2, or by at least 2.5, or by at least 3, or by at least 3.5, or by at least 4, or by at least 4.5, or by at least 5, or by at least 5.5,or by at least 6 or more standard deviations above a control level of sAPPβ reduction detected in the in vitro assay when the compound is tested at a concentration of about 0.2 μM, or about 2 μM, or about 2.2 μM, or about 10 μM, wherein such a reduction of sAPPβ correlates with a compound's therapeutic efficacy. In one embodiment, the control level of sAPPβ reduction may be the average sAPPβ level in control cell lines that are not contacted with the candidate compound. In other embodiments, the control level may be the average level of sAPPβ reduction achieved by a series of compounds tested in the in vitro assay.

In one preferred non-limiting embodiment, an effective amount of a compound of Formulas I-VII, and/or a compound depicted in FIG. 19, may be correlated with the compound's ability to reduce sAPPβ levels by about 4 standard deviations greater than a control level of sAPPβ reduction when the compound is administered at a concentration of 0.2 μM in the in vitro assay.

In other preferred non-limiting embodiments, an effective amount of a compound of Formulas I-VII, and/or a compound depicted in FIG. 19, may be correlated with the compound's ability to reduce the sAPPβ level by about 4 standard deviations greater than a control level of sAPPβ reduction when the compound is administered at a concentration of 2 μM in the in vitro assay.

In other preferred non-limiting embodiments, an effective amount of a compound of Formulas I-VII, and/or a compound depicted in FIG. 19, may be correlated with the compound's ability to reduce the sAPPβ level by about 4 standard deviations greater than a control level of sAPPβ reduction when the compound is administered at a concentration of 2.2 μM in the in vitro assay.

In other preferred non-limiting embodiments, an effective amount of a compound of Formulas I-VII, and/or a compound depicted in FIG. 19, may be that amount which reduces the sAPPβ level by about 4 standard deviations greater than a control level of sAPPβ reduction when the compound is administered at a concentration of 10 μM in the in vitro assay.

In another example, an effective amount of a compound of Formulas I-VII, and/or a compound depicted in FIG. 19, may be that amount which reduces the level of sAPPβ by at least 5%, by at least 10%, by at least 20%, by at least 30%, by at least 40%, by at least 50%, by at least 60%, by at least 70%, by at least 80%, by at least 90%, or by 100% when compared to sAPPβ level in a control cell line that was not contacted with the candidate compound, wherein to such a reduction of sAPPβ correlates with a compound's therapeutic efficacy.

In another example, an effective amount of a compound of Formulas I-VII, and/or a compound depicted in FIG. 19, may be that amount which reduces the level of sAPPβ by at least about 50% compared to a control cell line that was not contacted with the candidate compound. Preferably the compound is tested at a concentration ranging from about 200 μM to about 0.01 μM, preferably from about 100 μM to about 0.01 μM, and more preferably from about 10 μM to about 0.01 μM in the in vitro assay, wherein such a reduction of sAPPβ at the above-described concentrations is correlative with the compound's therapeutic efficacy.

In other preferred non-limiting embodiments, an effective amount of a compound of Formulas I-VII, and/or a compound depicted in FIG. 19, may be correlated with the compound's ability to reduce the sAPPβ level by about at least 50% when the compound is administered at a concentration of about 0.1 μM in the in vitro assay.

In other preferred non-limiting embodiments, an effective amount of a compound of Formulas I-VII, and/or a compound depicted in FIG. 19, may be correlated with the compound's ability to reduce the sAPPβ level by about at least 50% when the compound is administered at a concentration of about 0.5 μM in the in vitro assay.

In other preferred non-limiting embodiments, an effective amount of a compound of Formulas 1-VII, and/or a compound depicted in FIG. 19, may be correlated with the compound's ability to reduce the sAPPβ level by about at least 50% when the compound is administered at a concentration of about 1 μM in the in vitro assay.

In other preferred non-limiting embodiments, an effective amount of a compound of Formulas I-VII, and/or a compound depicted in FIG. 19, may be correlated with the compound's ability to reduce the sAPPβ level by about at least 50% when the compound is administered at a concentration of less than 5 μM in the in vitro assay.

In other preferred non-limiting embodiments, an effective amount of a compound of Formulas I-VII, and/or a compound depicted in FIG. 19, may be correlated with the compound's ability to reduce the sAPPβ level by about at least 50% when the compound is administered at a concentration of about 10 μM in the in vitro assay.

In other preferred non-limiting embodiments, an effective amount of a compound of Formulas I-VII, and/or a compound depicted in FIG. 19, may be correlated with the compound's ability to reduce the sAPPβ level by about at least 50% when the compound is administered at a concentration of about 15 μM in the in vitro assay.

In other preferred non-limiting embodiments, an effective amount of a compound of Formulas I-VII, and/or a compound depicted in FIG. 19, may be correlated with the compound's ability to reduce the sAPPβ level by about at least 50% when the compound is administered at a concentration of about 20 μM in the in vitro assay.

In other preferred non-limiting embodiments, an effective amount of a compound of Formulas I-VII, and/or a compound depicted in FIG. 19, may be correlated with the compound's ability to reduce the sAPPβ level by about at least 50% when the compound is administered at a concentration of about 25 μM in the in vitro assay.

In other non-limiting embodiments, the effective amount of at least one compound of Formulas I-VII, and/or a compound depicted in FIG. 19, may be correlated with the compound's ability to inhibit the enzymatic activity of a BACE1 enzyme. The compound's BACE1 inhibitory effect may be assayed, for example, through use of a BACE1 FRET Assay kit (Invitrogen Corp., Carlsbad, Calif., U.S.A.), wherein the fluorescence resonance energy transfer (FRET)-based assay measures the cleavage by purified recombinant β-secretase of a peptide substrate corresponding to the BACE1 cleavage site of Swedish mutant APP. In such an assay, a greater reduction in fluorescence in the reaction mixture following incubation with a compound of the invention compared to a control cell line not contacted with the compound is correlative with the compound's therapeutic efficacy.

In one example, an effective amount of a compound of Formulas I-VII, and/or a compound depicted in FIG. 19, may be that amount which inhibits BACE1 enzymatic activity by at least about 50% compared to a control cell that was not contacted with the candidate compound. Preferably the compound is tested at a concentration ranging from about 200 μM to about 0.01 μM, preferably from about 100 μM to about 0.01 μM, and more preferably from about 10 μM to about 0.01 μM in the in vitro assay, wherein such an inhibition of BACE1 enzymatic activity at the above-described concentrations is correlative with the compound's therapeutic efficacy.

In other preferred non-limiting embodiments, an effective amount of a compound of Formulas I-VII, and/or a compound depicted in FIG. 19, may be correlated with the compound's ability to inhibit BACE1 enzymatic activity by about at least 50% when the compound is administered at a concentration of about 2 μM in the in vitro assay.

In other preferred non-limiting embodiments, an effective amount of a compound of Formulas I-VII, and/or a compound depicted in FIG. 19, may be correlated with the compound's ability to inhibit BACE1 enzymatic activity by about at least 50% when the compound is administered at a concentration of about 2.5 μM in the in vitro assay.

In other preferred non-limiting embodiments, an effective amount of a compound of Formulas I-VII, and/or a compound depicted in FIG. 19, may be correlated with the compound's ability to inhibit BACE1 enzymatic activity by about at least 50% when the compound is administered at a concentration of about 5 μM in the in vitro assay.

In other preferred non-limiting embodiments, an effective amount of a compound of Formulas I-VII, and/or a compound depicted in FIG. 19, may be correlated with the compound's ability to inhibit BACE1 enzymatic activity by about at least 50% when the compound is administered at a concentration of about 5.5 μM in the in vitro assay.

In other preferred non-limiting embodiments, an effective amount of a compound of Formulas I-VII, and/or a compound depicted in FIG. 19, may be correlated with the compound's ability to inhibit BACE1 enzymatic activity by about at least 50% when the compound is administered at a concentration of about 6 μM in the in vitro assay.

In other non-limiting embodiments, the effective amount of at least one compound of Formulas I-VII, and/or a compound depicted in FIG. 19, may be correlated with the compound's ability to reduce the level of Aβ₄₀ in an in vitro assay that measures the level of Aβ₄₀ produced in a cell line, for example, an SY5Y-BACEGFP-SEAPAPPwt cell line. In one non-limiting example, the level of Aβ₄₀ expressed by the cell line may be measured through the use of an Aβ₄₀ Elisa kit (Bio Source). In one embodiment, the assay comprises incubating the Aβ₄₀ expressing cells with a compound of Formulas I-VII, and/or a compound depicted in FIG. 19, followed by assaying the concentration of Aβ₄₀ in the cell media. In such an assay, a greater reduction of Aβ₄₀ concentration in the cell media following incubation with a compound compared to a control cell line not contacted with the compound is correlative with the compound's therapeutic efficacy.

In one example, an effective amount of a compound of Formulas I-VII, and/or a compound depicted in FIG. 19, may be that amount which reduces the level of Aβ₄₀ in a cell in an in vitro assay by at least about 50% compared to a control cell line that was not contacted with the candidate compound. Preferably the compound is tested at a concentration ranging from about 200 μM to about 0.01 μM, preferably from about 100 μM to about 0.01 μM, and more preferably from about 10 μM to about 0.01 μM in the in vitro assay, wherein such a reduction in the level of Aβ₄₀ at the above-described concentrations is correlative with the compound's therapeutic efficacy.

In other preferred non-limiting embodiments, an effective amount of a compound of Formulas I-VII, and/or a compound depicted in FIG. 19, may be correlated with the compound's ability to reduce the level of Aβ₄₀ in the in vitro assay by about at least 50% when the compound is administered at a concentration of about 0.05 μM in the in vitro assay.

In other preferred non-limiting embodiments, an effective amount of a compound of Formulas I-VII, and/or a compound depicted in FIG. 19, may be correlated with the compound's ability to reduce the level of Aβ₄₀ in the in vitro assay by about at least 50% when the compound is administered at a concentration of about 0.1 μM in the in vitro assay.

In other preferred non-limiting embodiments, an effective amount of a compound of Formulas I-VII, and/or a compound depicted in FIG. 19, may be correlated with the compound's ability to reduce the level of Aβ₄₀ in the in vitro assay by about at least 50% when the compound is administered at a concentration of about 1 μM in the in vitro assay.

In other preferred non-limiting embodiments, an effective amount of a compound of Formulas I-VII, and/or a compound depicted in FIG. 19, may be correlated with the compound's ability to reduce the level of Aβ₄₀ in the in vitro assay by about at least 50% when the compound is administered at a concentration of about 2 μM in the in vitro assay.

In other preferred non-limiting embodiments, an effective amount of a compound of Formulas I-VII, and/or a compound depicted in FIG. 19, may be correlated with the compound's ability to reduce the level of Aβ₄₀ in the in vitro assay by about at least 50% when the compound is administered at a concentration of about 5 μM in the in vitro assay.

In other preferred non-limiting embodiments, an effective amount of a compound of Formulas I-VII, and/or a compound depicted in FIG. 19, may be correlated with the compound's ability to reduce the level of Aβ₄₀ in the in vitro assay by about at least 50% when the compound is administered at a concentration of about 5.5 μM in the in vitro assay.

In other preferred non-limiting embodiments, an effective amount of a compound of Formulas I-VII, and/or a compound depicted in FIG. 19, may be correlated with the compound's ability to reduce the level of Aβ₄₀ in the in vitro assay by about at least 50% when the compound is administered at a concentration of about 6 μM in the in vitro assay.

In other preferred non-limiting embodiments, an effective amount of a compound of Formulas I-VII, and/or a compound depicted in FIG. 19, may be correlated with the compound's ability to reduce the level of Aβ₄₀ in the in vitro assay by about at least 50% when the compound is administered at a concentration of about 6.5 μM in the in vitro assay.

In other preferred non-limiting embodiments, an effective amount of a compound of Formulas I-VII, and/or a compound depicted in FIG. 19, may be correlated with the compound's ability to reduce the level of Aβ₄₀ in the in vitro assay by about at least 50% when the compound is administered at a concentration of about 7 μM in the in vitro assay.

In other preferred non-limiting embodiments, an effective amount of a compound of Formulas I-VII, and/or a compound depicted in FIG. 19, may be correlated with the compound's ability to reduce the level of Aβ₄₀ in the in vitro assay by about at least 50% when the compound is administered at a concentration of about 8 μM in the in vitro assay.

In other non-limiting embodiments, the effective amount of at least one compound of Formulas I-VII, and/or at least compound depicted in FIG. 19, may be correlated with the compound's ability to reduce the level of Aβ₄₀ in an in vitro assay that measures the level of Aβ₄₀ produced in a cell culture, for example, a culture of primary cortical neurons transduced with lentivirus carrying Swedish mutant APP (APPsw). In one embodiment, the assay comprises incubating the Aβ₄₀ expressing cells with a compound of Formulas I-VII, and/or a compound depicted in FIG. 19, followed by assaying the concentration of Aβ₄₀ in the cell culture medium. In such an assay, a greater reduction of Aβ₄₀ concentration in the cell culture medium following incubation with a compound compared to a control cell culture not contacted with the compound is correlative with the compound's therapeutic efficacy.

In one non-limiting embodiment, an effective amount of a compound of Formulas I-VII, and/or a compound depicted in FIG. 19, may be that amount which reduces Aβ₄₀ by about 1-10%, more preferably from about 10-20%, more preferably from about 20-30%, more preferably from about 30-40%, more preferably from about 40-50%, more preferably from about 50-60%, more preferably from about 60-70%, more preferably from about 70-80%, more preferably from about 80-90%, and more preferably from about 90-100%, compared to Aβ₄₀ levels in the cell media of a control cell culture that was not incubated with the compound, when the compound is incubated at a concentration of about 200 μM to about 0.01 μM, preferably from about 100 μM to about 0.01 μM, and more preferably from about 10 μM to about 0.01 μM in the in vitro assay, wherein a greater level of Aβ₄₀ reduction at a lower concentration in the in vitro assay is correlative with the compound's therapeutic efficacy.

In one preferred embodiment, the compound is incubated with the cell line in the in vitro assay at a concentration of about 10 μM, and the level of Aβ₄₀ is reduced by at least about 15% compared to Aβ₄₀ levels in the cell media of a control cell line that was not incubated with the compound.

In other preferred embodiments, the compound is incubated with the cell line in the in vitro assay at a concentration of about 15 μM, and the level of Aβ₄₀ is reduced by at least about 40% compared to Aβ₄₀ levels in the cell media of a control cell line that was not incubated with the compound.

In other preferred embodiments, the compound is incubated with the cell line in the in vitro assay at a concentration of about 20 μM, and the level of Aβ₄₀ is reduced by at least about 75% compared to Aβ₄₀ levels in the cell media of a control cell line that was not incubated with the compound.

In other preferred embodiments, the compound is incubated with the cell line in the in vitro assay at a concentration of about 5 μM, and the level of Aβ₄₀ is reduced by at least about 80% compared to Aβ₄₀ levels in the cell media of a control cell line that was not incubated with the compound.

In other non-limiting embodiments, the effective amount of at least one compound of Formulas I-VII, and/or a compound depicted in FIG. 19, may be correlated with the compound's ability to reduce the level of sAPPβ in an in vitro assay that measures the level of sAPPβ produced in a cell line, for example, primary cortical neurons transduced with lentivirus carrying Swedish mutant APP (APPsw). In one embodiment, the assay comprises incubating the cells with a compound of Formulas I-VII, and/or a compound depicted in FIG. 19, followed by assaying the concentration of sAPPβ in the cell culture medium. In such an assay, a greater reduction of sAPPβ concentration in the cell culture medium following incubation with a compound compared to a control cell culture not contacted with the compound is correlative with the compound's therapeutic efficacy.

In one non-limiting embodiment, an effective amount of a compound of Formulas I-VII, and/or a compound depicted in FIG. 19, may be that amount which reduces sAPPβ by about 1-10%, more preferably from about 10-20%, more preferably from about 20-30%, more preferably from about 30-40%, more preferably from about 40-50%, more preferably from about 50-60%, more preferably from about 60-70%, more preferably from about 70-80%, more preferably from about 80-90%, and more preferably from about 90-100%, compared to sAPPβ levels in the cell culture medium of a control cell culture that was not incubated with the compound. Preferably the compound is incubated at a concentration of about 200 μM to about 0.01 μM, preferably from about 100 μM to about 0.01 μM, and more preferably from about 10 μM to about 0.01 μM in the in vitro assay, wherein a greater level of sAPPβ reduction at a lower concentration in the in vitro assay is correlative with the compound's therapeutic efficacy.

In one preferred embodiment, the compound is incubated with the cell line in the in vitro assay at a concentration of about 20 μM, and the level of sAPPβ is reduced by at least about 40% compared to sAPPβ levels in the cell media of a control cell line that was not incubated with the compound.

In other non-limiting embodiments, an effective amount of a compound of Formulas I-VII, and/or a compound depicted in FIG. 19, may be that amount which reduces Aβ₄₀ by about 50% in an in vitro assay compared to Aβ₄₀ levels in the cell culture medium of a control cell culture that was not incubated with the compound. Preferably the compound is incubated at a concentration of about 200 μM to about 0.01 μM, preferably from about 100 μM to about 0.01 μM, and more preferably from about 10 μM to 0.01 μM in the in vitro assay, wherein a reduction of Aβ₄₀ at a lower concentration in the in vitro assay is correlative with the compound's therapeutic efficacy.

In one non-limiting-embodiment, the level of Aβ₄₀ is reduced by about 50% when the compound is incubated with the cell line in the in vitro assay at a concentration of about 6 μM.

In other non-limiting embodiments, the level of Aβ₄₀ is reduced by about 50% when the compound is incubated with the cell line in the in vitro assay at a concentration of about 3.5 M.

In other non-limiting embodiments, the effective amount of at least one compound of Formulas I-VII, and/or at least one compound depicted in FIG. 19, may be correlated with the compound's ability to reduce the level of Aβ₄₀ in an in vitro assay that measures the level of Aβ₄₀ produced in a cell line, for example, cultured primary cortical neurons prepared from Tg2576 mice (i.e. mice carrying human APPsw transgene under the control of the PrP promoter). In one embodiment, the assay comprises incubating the Aβ₄₀ expressing cells with a compound of Formulas I-VII, and/or a compound depicted in FIG. 19, followed by assaying the concentration of Aβ₄₀ in the cell media. In such an assay, a greater reduction of Aβ₄₀ concentration in the cell media following incubation with a compound compared to a control cell line not contacted with the compound is correlative with the compound's therapeutic efficacy.

In one non-limiting embodiment, an effective amount of a compound of Formulas I-VII, and/or a compound depicted in FIG. 19, may be that amount which reduces Aβ₄₀ by about 1-10%, more preferably from about 10-20%, more preferably from about 20-30%, more preferably from about 30-40%, more preferably from about 40-50%, more preferably from about 50-60%, more preferably from about 60-70%, more preferably from about 70-80%, more preferably from about 80-90%, and more preferably from about 90-100%, compared to Aβ₄₀ levels in the cell culture medium of a control cell culture that was not incubated with the compound. Preferably the compound is incubated at a concentration of about 200 μM to about 0.01 μM, preferably from about 100 μM to about 0.01 nM, and more preferably from about 10 μM to about 0.01 μM in the in vitro assay, wherein a greater level of Aβ₄₀ reduction at a lower concentration in the in vitro assay is correlative with the compound's therapeutic efficacy.

In one preferred embodiment, the compound is incubated with the cell line in the in vitro assay at a concentration of about 20 μM, and the level of Aβ₄₀ is reduced by at least about 65% compared to Aβ₄₀ levels in the cell media of a control cell line that was not incubated with the compound.

In other preferred embodiments, the compound is incubated with the cell line in the in vitro assay at a concentration of about 5 μM, and the level of Aβ₄₀ is reduced by at least about 60% compared to Aβ₄₀ levels in the cell media of a control cell line that was not incubated with the compound.

In other non-limiting embodiments, the effective amount of at least one compound of Formulas I-VII, and/or at least one compound depicted in FIG. 19, may be correlated with the compound's ability to reduce the level of Aβ₄₀ and/or sAPPβ in an ex vivo assay that measures the level of Aβ₄₀ and/or sAPPβ produced in cells, for example, organotypic brain slices from Tg2576 mice (i.e. mice carrying human APPsw transgene under the control of the PrP promoter). In one embodiment, the assay comprises incubating the brain slices with a compound of Formulas I-VII, and/or a compound depicted in FIG. 19, followed by assaying the concentration of Aβ₄₀ and/or sAPPβ in the brain slices. In such an assay, a greater reduction of Aβ₄₀ and/or sAPPβ concentration in the brain slices following incubation with a compound compared to a control brain slice not contacted with the compound is correlative with the compound's therapeutic efficacy.

In one non-limiting embodiment, an effective amount of a compound of Formulas I-VII, and/or a compound depicted in FIG. 19, may be that amount which reduces Aβ₄₀ and/or sAPPβ by about 1-10%, more preferably from about 10-20%, more preferably from about 20-30%, more preferably from about 30-40%, more preferably from about 40-50%, more preferably from about 50-60%, more preferably from about 60-70%, more preferably from about 70-80%, more preferably from about 80-90%, and more preferably from about 90-100%, compared to Aβ₄₀ and/or sAPPβ levels in control brain slices that were not incubated with the compound. Preferably the compound is incubated at a concentration of about 200 μM to about 0.01 μM, preferably from about 100 μM to about 0.01 μM, and more preferably from about 10 μM to about 0.01 μM in the ex vivo assay, and a greater level of Aβ₄₀ and/or sAPPβ reduction at a lower concentration in the ex vivo assay is correlative with the compound's therapeutic efficacy.

In other non-limiting embodiments, the effective amount of at least one compound of Formulas I-VII, and/or at least one compound depicted in FIG. 19, may be correlated with the compound's ability to reduce the level of Aβ₄₀ and/or sAPPβ in an in vivo assay that measures the level of Aβ₄₀ and/or sAPPβ produced in a test subject, for example, a Tg2576 mouse (i.e. mice carrying human APPsw transgene under the control of the PrP promoter). In one embodiment, the assay comprises administering a compound of Formulas I-VII, and/or a compound depicted in FIG. 19, to the test subject, for example, via interstitial fluid (ISF) compound administration, intraperitoneal (IP) compound injection, or through the use of a microdialysis apparatus for infusion of the compound at multiple concentrations (for example, in the hippocampus of the test subject), followed by assaying the concentration of Aβ₄₀ and/or sAPPβ in the test subject. In such an assay, a greater reduction of Aβ₄₀ and/or sAPPβ concentration in the test subject following administration of the compound compared to a control subject not administered the compound is correlative with the compound's therapeutic efficacy.

In one non-limiting embodiment, an effective amount of a compound of Formulas I-VII, and/or a compound depicted in FIG. 19, may be that amount which reduces Aβ₄₀ and/or sAPPβ by about 1-10%, more preferably from about 10-20%, more preferably from about 20-30%, more preferably from about 30-40%, more preferably from about 40-50%, more preferably from about 50-60%, more preferably from about 60-70%, more preferably from about 70-80%, more preferably from about 80-90%, and more preferably from about 90-100%, compared to Aβ₄₀ and/or sAPPβ levels in brain homogenates of subjects that were not administered the compound. Preferably the compound is administered at a concentration of about 0.5 mg/kg to about 20 mg/kg, preferably from about 1 mg/kg to about 20 mg/kg, more preferably from about 3 mg/kg to about 20 mg/kg, more preferably from about 5 mg/kg to about 20 mg/kg, more preferably from about 10 mg/kg to about 20 mg/kg in the in vivo assay, and a greater level of Aβ₄₀ and/or sAPPβ reduction at a lower concentration in the in vivo assay is correlative with the compound's therapeutic efficacy.

In one preferred embodiment, the compound is administered in the in vivo assay at a concentration of about 3 mg/kg, and the level of Aβ₄₀ is reduced by at least about 30% compared to Aβ₄₀ levels in brain homogenate of a control subject that was not administered the compound.

In non-limiting embodiments, an effective amount of a compound of Formulas I-VII, and/or a compound depicted in FIG. 19, may be an amount which achieves a local concentration at the therapeutic site of about 100 μM to about 0.01 μM, preferably from about 50 μM to about 0.01 μM, more preferably from about 20 μM to about 0.01 μM, and more preferably from about 10 μM to about 0.01 μM in the in vitro assay.

### 5.3.2 Administration of Treatments

According to the invention, the component or components of a pharmaceutical composition of the invention may be administered by, for example and not by way of limitation, intravenous, intra-arterial, intramuscular, intradermal, transdermal, subcutaneous, oral, intraperitoneal, intraventricular, and intrathecal administration.

In particular non-limiting embodiments, the therapeutic compound can be delivered in a controlled or sustained release system. For example, a compound or composition may be administered using intravenous infusion, an implantable osmotic pump, a transdermal patch, liposomes, or other modes of administration. In one embodiment, a pump may be used (see Sefton, 1987, CRC Crit. Ref Biomed. Eng. 14:201; Buchwald et al., 1980, Surgery 88:507; Saudek et al., 1989, N. Engl. J. Med. 321:574). In another embodiment, polymeric materials can be used (see Langer and Wise eds., 1974, Medical Applications of Controlled Release, CRC Press: Boca Raton, Fla.; Smolen and Ball eds., 1984, Controlled Drug Bioavailability, Drug Product Design and Perfamiance, Wiley, N.Y.; Ranger and Peppas, 1983, J. Macromol. Sci. Rev. Macromol. Chem., 23:61; Levy et al., 1985, Science 228:190; During et al., 1989, Ann. Neurol., 25:351; Howard et al., 9189, J. Neurosurg. 71:105). In yet another embodiment, a controlled release system can be placed in proximity of the therapeutic target, i.e., the heart or a blood vessel, thus requiring only a fraction of the systemic dose (see, e.g., Goodson, 1984, in Medical Applications of Controlled Release, supra, Vol. 2, pp. 115-138). Other controlled release systems known in the art may also be used.

### 5.4 Pharmaceutical Compositions

The compounds and compositions of the invention may be formulated as pharmaceutical compositions by admixture with a pharmaceutically acceptable carrier or excipient.

In one non-limiting embodiment, the pharmaceutical composition may comprise an effective amount of at least one compound of Formulas I-VII, and/or at least one compound depicted in FIG. 19, and a physiologically acceptable diluent or carrier. The pharmaceutical composition may further comprise a second drug, for example, but not by way of limitation, a compound for the treatment of Alzheimer's disease, such as an acetylcholinesterase inhibitor or an NMDA glutamate receptor antagonist (e.g. memantine).

The phrase “pharmaceutically acceptable” refers to substances that are physiologically tolerable when administered to a subject. Preferably, but not by way of limitation, as used herein, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, or, for solid dosage forms, may be standard tabletting excipients. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition, or other editions.

In a specific embodiment, the therapeutic compound can be delivered in a vesicle, in particular a liposome (see Langer, 1990, Science 249:1527-1533; Treat et al., 1989, in Liposomes in the Therapy of Infectious Disease and Cancer, Lopez-Berestein and Fidler eds., Liss: New York, pp. 353-365; Lopez-Berestein, ibid., pp. 317-327; see generally Lopez-Berestein, ibid.).

### EXAMPLES

### Example 1

### High- and Medium-Throughput Screening of Small Molecule Libraries to Identify Small Molecule Modulators of BACE1

A cell-based modified ELISA assay for measuring sAPPβ, the secreted ectodomain of β-amyloid precursor protein (APP) following β-secretase (BACE1) cleavage, was used to identify a class of compounds that interfered with the first step of sAPPβ generation. This assay has been described in International Application PCT/US2007/015938 (Published as International Publication No. WO 08/008463), which is herein incorporated in its entirety for all purposes.

BACE1-mediated cleavage of APP is a key and necessary event in-the generation of neurotoxic β-amyloid (Aβ), a widely accepted contributor to the development of Alzheimer's disease (AD). Studies in BACE1 knockout mice showed that they are viable, fertile, and do not produce Aβ, making BACE1 an attractive target for AD therapeutic intervention.

The SY5Y-BACEGFP-SEAPAPPwt cell based assay was developed to discover novel small molecule modulators of BACE1 activity. SY5Y cells were stably transfected with GFP-tagged BACE1 (BACE-GFP) and secreted alkaline phosphatase (SEAP)-tagged wildtype APP (SEAP-APPwt). BACE1 cleavage of SEAP-APPwt results in secretion into the media of SEAP-tagged sAPPβ, which is collected and specifically captured using an sAPP0 cleavage site-specific antibody (sβwt). After washing, the fluorescent alkaline phosphatase substrate 4-methylumbelliferyl phosphate (4-MUP) is used to detect the captured SEAP-sAPPβ (FIG. 1).

Two chemical libraries were screened using the SY5Y-BACEGFP-SEAPAPPwt cell based assay: a tagged-triazine-based chemical library of nearly 3,000 compounds (Khersonsky et al., 2003); and the Laboratory for Drug Discovery in Neurodegeneration's at Harvard University (hereinafter “LDDN”) collection of nearly 140,000 small molecule compounds. High- and medium-throughput screening of these compound libraries identified numerous potent small molecules capable of reducing sAPPβ, and the results were confumed in numerous secondary assays using the screening cell line. Several chemotypes were identified that were potent inhibitors of sAPPβ in SY5Y-BACEGFP-SEAPAPPwt cells.

### Tagged-Triazine Compound Library

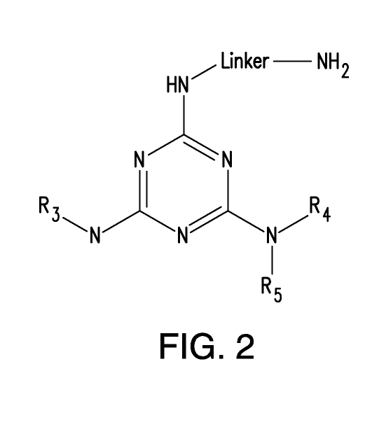

The triazine scaffold was selected for combinatorial synthesis due to its ease of manipulation and structural similarity to purine and pyrimidine, which have already demonstrated activity in several biological systems (Chang et al., 2002, Chembiochem, 3, 897-901; Verdugo et al., 2001, J Med Chem, 44, 2683-2686; Armstrong et al., 2000, Chem Int Edit, 39, 1303-1306; Rosania et al., 2000, Nat Biotechnol, 18, 304-308; Chang et al., 1999, Chemistry & Biology, 6, 361- 375; Gangjee et al., 2003, J Med Chem, 46, 591-600; Baraldi et al., 1998, J Med Chem, 41, 2126-2133; and Baraldi et al., 2002, J Med Chem, 45, 115-126). The triazine scaffold has three-fold symmetry, and each compound in the combinatorial library contains a built-in linker moiety for easy attachment of an affinity bead (FIG. 2). This, in effect, bypasses the traditional (and time-consuming) structure-activity relationship (SAR) studies required for attachment of a fluorescent or purification tag.

The primary screen for the nearly 3,000-compound tagged-triazine library was conducted in 96-well format by hand. Compounds were supplied in powder form in 96-well polypropylene plates. The compounds were first dissolved in DMSO to generate 10 mM stock plates, then stamped onto new polypropylene plates to make 1 mM working stock solution. For assay, 1 mM working stock solution was diluted 1:100 into cell culture media for a final concentration of 10 μM. The resulting 1% DMSO concentration did not result in any cytotoxicity as measured by a standard MTS-based cell viability assay. Negative (DMSO) and positive (BACE inhibitor IV at 10 μM) controls were included on every screening plate in triplicate for quality control and calculation of Z′ factors. In addition, “zero” controls (media that has not been used for cell incubation) were included for all sAPPβ ELISA plates to measure the BACE1 assay background.

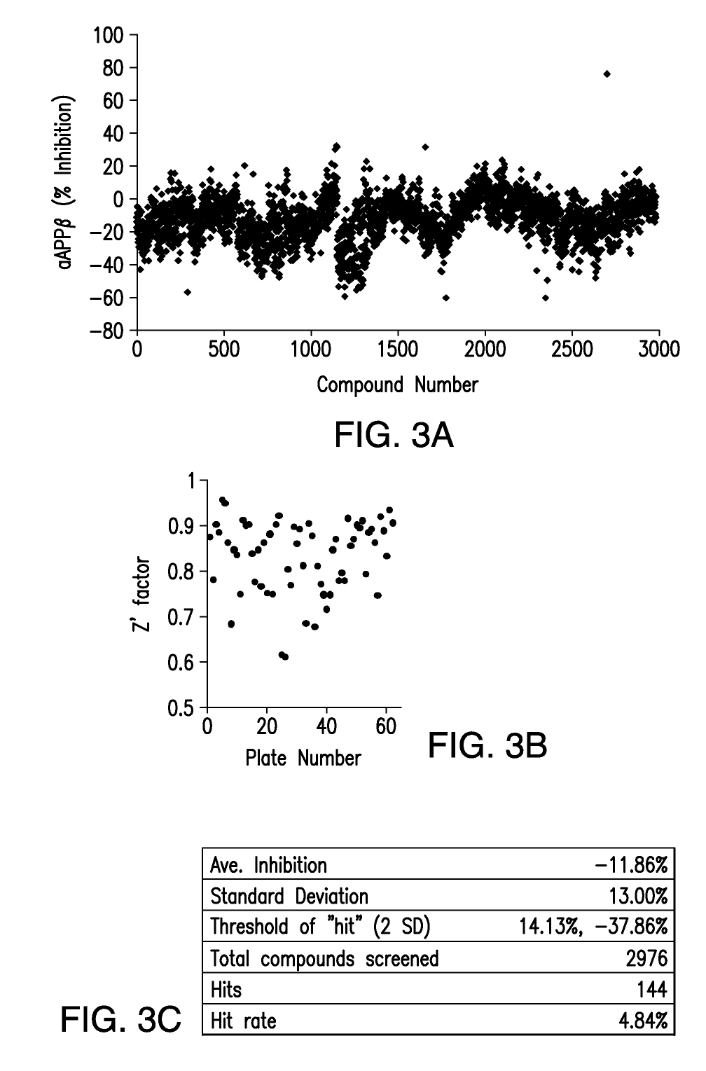

The BACE1 assay was performed as described, and data from the primary screen are shown in FIG. 3. Assay background was subtracted from all data points. Percent inhibition of the fluorescence signal was calculated as 100×{DMSO control−compound]/DMSO control. The majority of data points were clustered within roughly 30 percentage points, with a small number of outliers (FIG. 3A). The Z′ factors from the screen were excellent, as most plates achieved Z′ factors >0.7, and all were above the 0.5 threshold for an excellent assay. Mean±s.d. values of Z′ were 0.83±0.08 (0.61≦Z′≦0.96), with a median of 0.86 (FIG. 3B). Mean inhibition of the sAPPβ fluorescence signal was −11.86%, with a standard deviation of 13.00% (FIG. 3C). Due to the relatively small size of the compound library, and the fact that each compound was screened only once, the threshold value for hit selection was set at 2 standard deviations above and below the mean (or 14.13% and −37.86%) to generate more hits. Of 2976 compounds screened, 144 were identified as hits, for a hit rate of 4.84%.

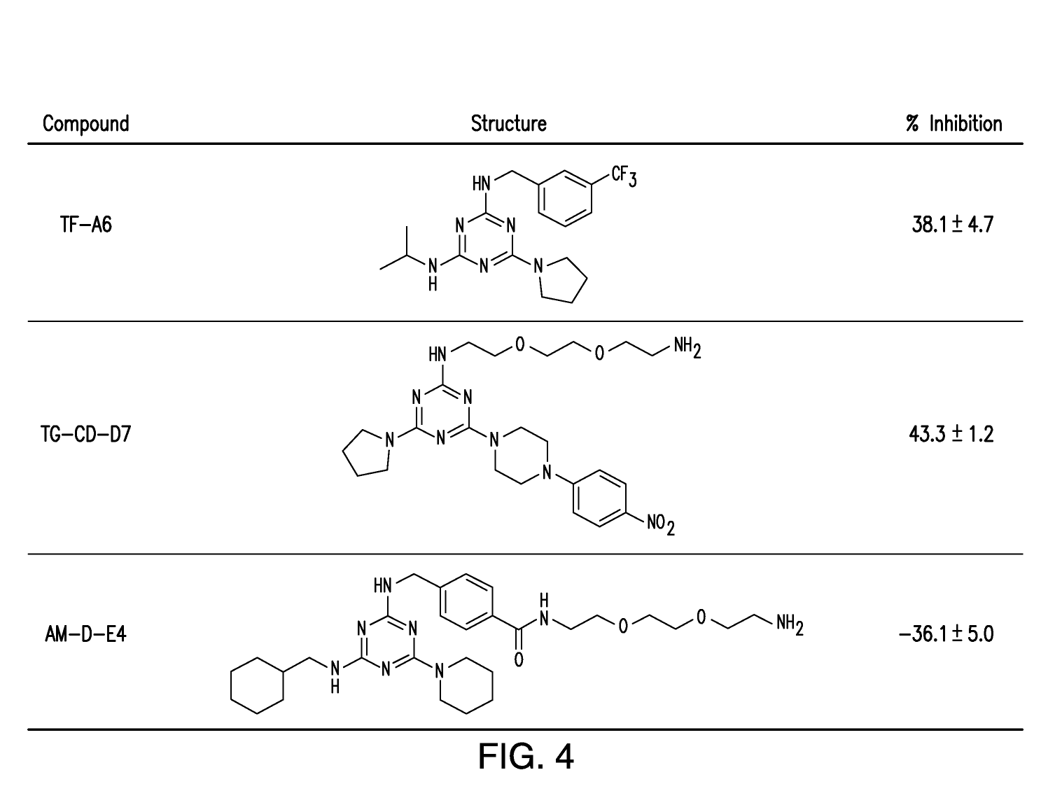

The 144 hits from the primary screen were rescreened in triplicate at 10 μM to confirm the activity and to assess cytotoxicity. Because of the low threshold used for hit selection, only 3 compounds reconfirmed (FIG. 4). Two (TF-A6 and TG-CD-D7) caused a modest inhibition of sAPPβ (38% and 43%, respectively), while one (AM-D-E4) resulted in a modest increase in sAPPβ (36%). The three compounds did not affect cell viability (data not shown).

Although 3 hits were successfully confirmed from the tagged-triazine library screen, these compounds display relatively low potency. In high-throughput chemical screening projects, one generally desires compound hits that have IC₅₀ values between 10 and 1 μM or better before commencing medicinal chemistry and structure-activity relationship studies.

### Compound Library of the Laboratory for Drug Discovery in Neurodegeneration

The LDDN library (NIH Molecular Libraries Small Molecule Repository) was screened through automation and miniaturization of the SY5Y-BACEGFP-SEAPAPPwt cell based assay. The BACE1 assay was automated and miniaturized from a 96-well assay down to a 384-well format. The compound library of the LDDN consists of roughly 140,000 small molecules, including compounds approved by the FDA, a purified natural products library, compounds purchased from various commercial sources, small molecules obtained from academic institutions, as well as those synthesized by LDDN chemists. To generate the library, compounds were selected from various sources based on a series of filters. Small molecules generally adhere to Lipinski's rules (Lipinski, 2000, J Pharm Tox Methods, 44, 235-249), which are a set of physicochemical properties that aid in the prediction of “drug-like” molecules. Some of these properties include molecular weight, the presence or absence of hydrogen bond donors and acceptors, and the hydrophobicity/hydrophilicity of the compound. In addition, known toxicophores as well as commonly unwanted functionalities, such as Michael acceptors, were filtered out to the best of the chemists' abilities.

The primary screen was conducted in 384-well format with the assistance of robotic workstations. Briefly, 0.4 μl of each compound (1.67 mM) dissolved in DMSO was diluted with 30 μl cell culture media to reach an intermediate concentration of 22 μM. SY5Y-BACEGFP-SEAPAPPwt cells were washed with 500 μl PBS with an automated plate washer under gentle washing conditions to minimize cell detachment, and 45 μl of cell culture media was added with the Multidrop liquid dispenser (Thermo Scientific). 5 μl of culture media containing 22 μM compound was transferred to the cell culture plate with the Biomek FX Laboratory Automation Workstation (Beckman Coulter) for a final screening concentration of 2.2 μM. Each plate contained negative (DMSO) and positive (BACE inhibitor IV at 10 μM) controls, each occupying 16 wells on the 384-well plate.

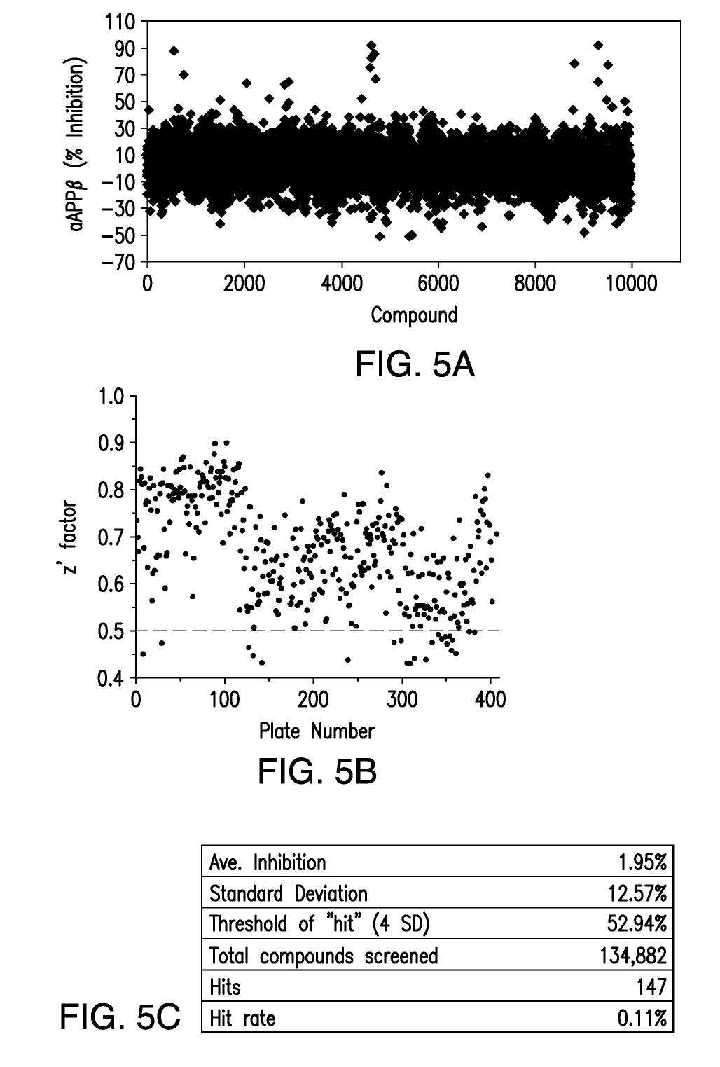

Data from the primary screen of the LDDN library are presented in FIG. 5. Data points were managed and analyzed by ActivityBase software. Because 10 μM BACE inhibitor IV was sufficient to suppress sAPPβ generation by >95% on average, the mean fluorescence signal from wells incubated with the inhibitor was taken as a close approximate of the BACE1 assay background and subtracted from all data points on the 384-well plate. Percent inhibition was then calculated as 100×[DMSO control−compound]/DMSO control. A representative 10,000 data points were plotted in FIG. 5A. The vast majority of compounds showed tight clustering between roughly -30% and 30% inhibition, while only a small percentage of compounds stood out from the noise. Z′ factors were high for the majority of plates screened (FIG. 5B). The majority of Z′ factors exceeded the threshold of 0.5 for an excellent assay. Mean±s.d. values of Z′ were 0.67±0.11 (0.43≦Z′≦0.90), with a median value of 0.67. Roughly 20 plates showed increased variability in the negative or positive control wells, lowering the Z′ factor to between 0.4 and 0.5. Mean inhibition of the sAPPβ fluorescence signal was 1.95%, with a standard deviation of 12.57% (FIG. 5C). The threshold for hit selection was set at 4 standard deviations above the mean, or 52.94%. Of 134,882 compounds screened, 147 registered as hits, for a hit rate of 0.11%. sAPPβ enhancers were not selected as hits due to the large number of hit compounds, and because early confirmation experiments showed a 0% confirmation rate for enhancer hits.

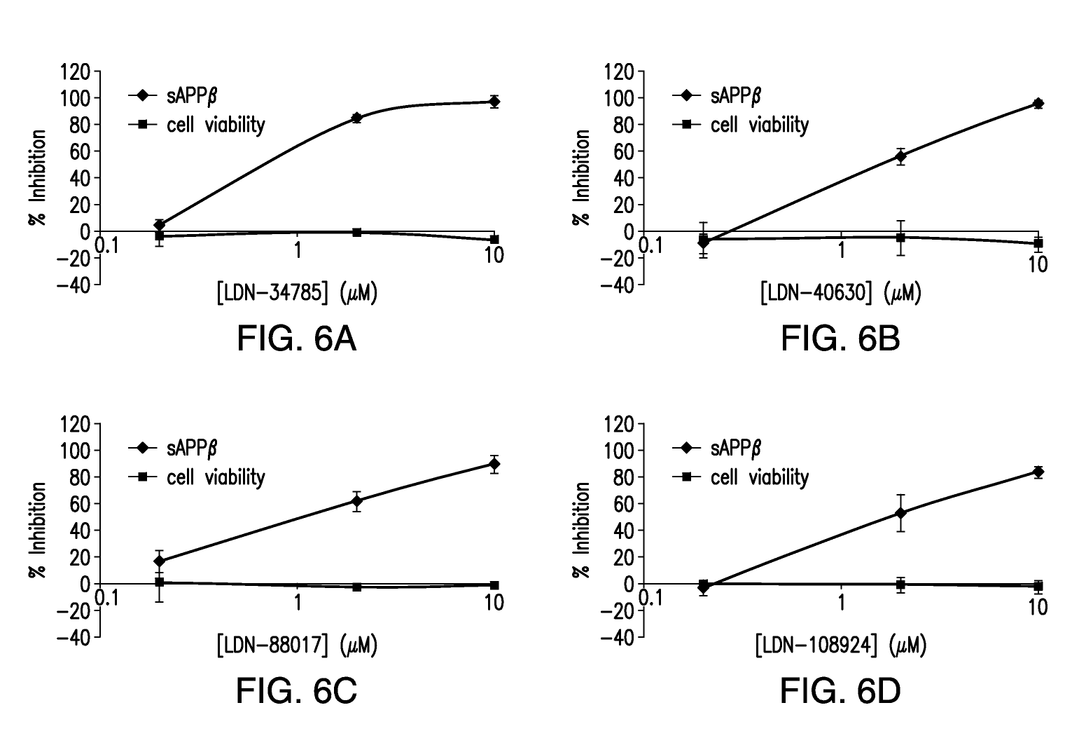

Of the 147 hits from the primary screen, 139 were retested for 3-point dose-response at 10, 2, and 0.2 μM concentrations in quadruplicate. 86 compounds were confirmed to have dose-responsive activity, for a confirmation rate of 62%. The compounds were simultaneously evaluated for cytotoxicity using the Cell Titer AQᵤₑₒᵤₛ One cell proliferation assay (Promega). Representative 3-point dose-response data from four compounds are shown in FIG. 6. Briefly, hit compounds were hand-picked from stock plates and transferred to 96-well polypropylene plates (24 compounds per plate). Complete media was added to make a 10 μM solution, then a serial dilution was performed to make 2 μM and 0.2 μM solutions in adjacent wells with the aid of the Biomek FX workstation. The full 96-well compound plate was then “quad-mapped” to a 384-well plate prior to transfer onto 384-well cell culture plates containing SY5Y-BACEGFP-SEAPAPPwt cells. Each plate of compounds was loaded onto two cell culture plates, one for BACE1 assay and the other for cell viability studies. The four compounds shown in FIG. 6 all exhibit dose-responsive inhibitory activity on sAPPβ while having no effect on cell viability.

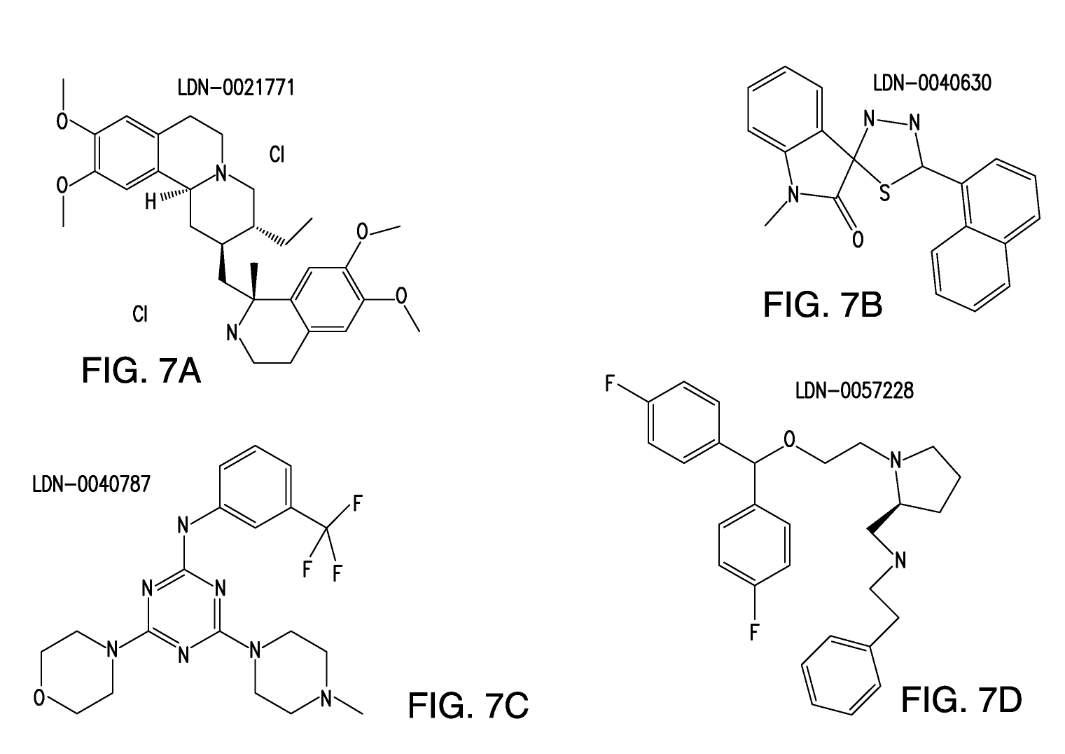

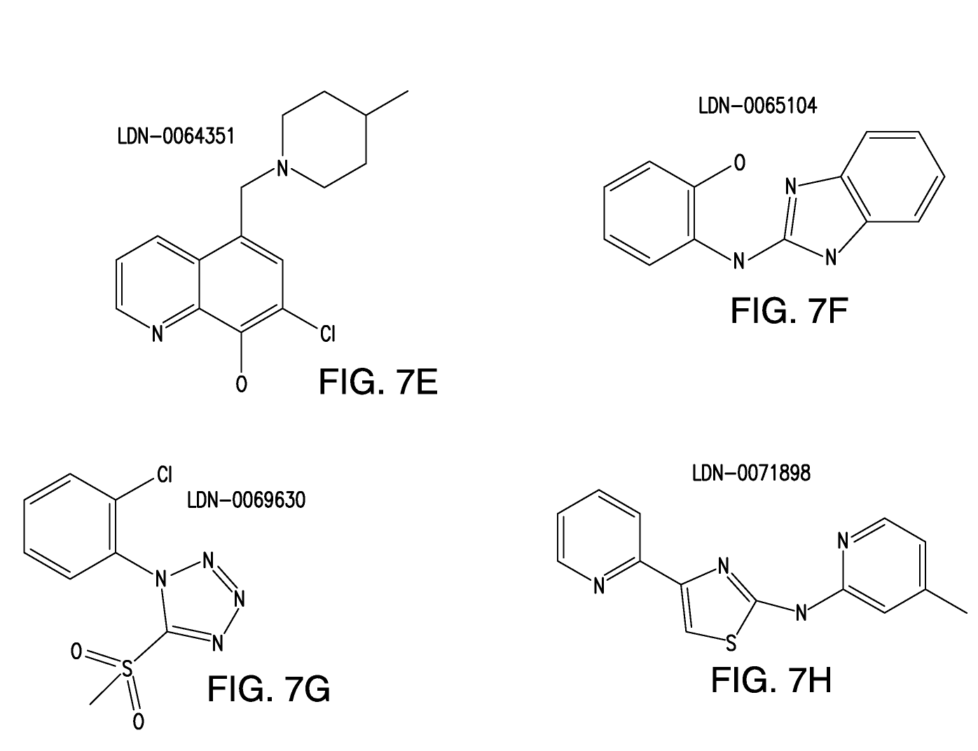

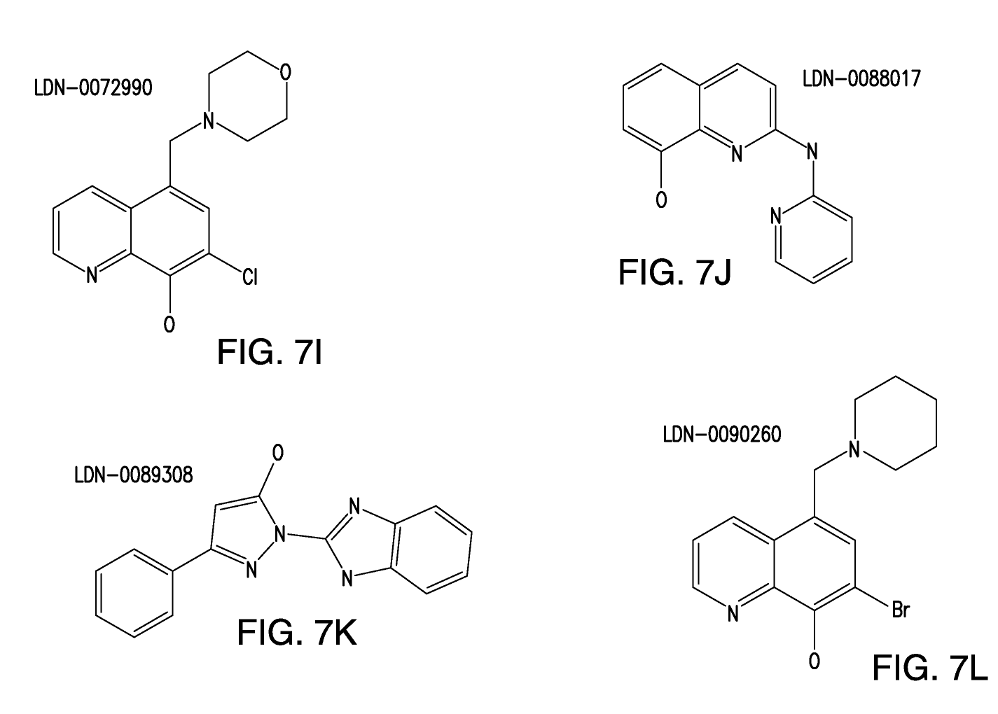

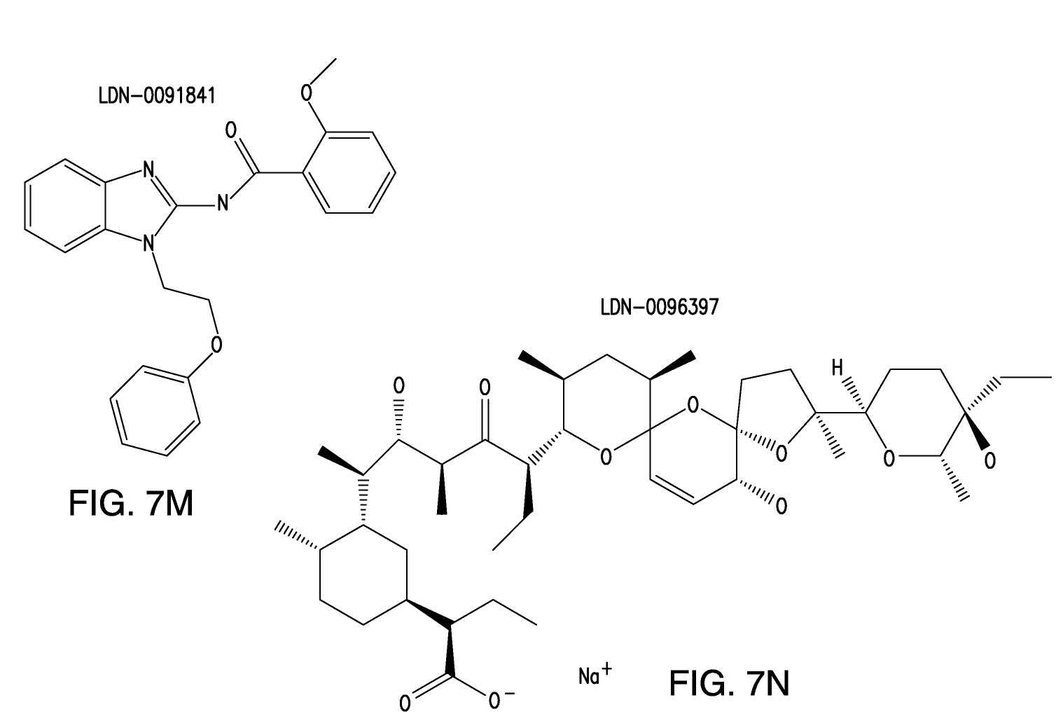

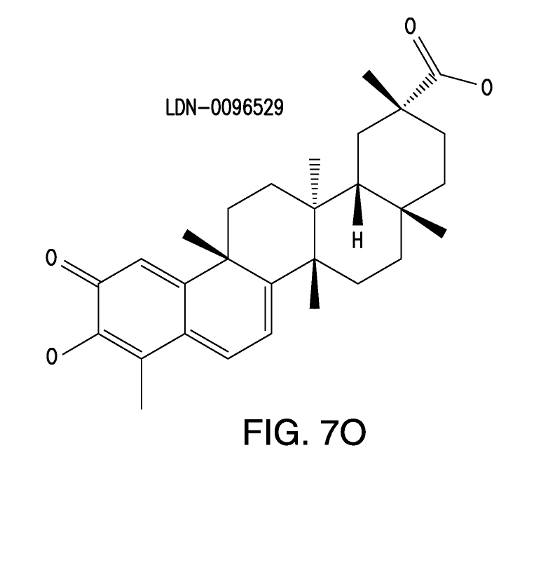

Based on their 3-point dose-response profile (estimated potency and lack of cytotoxicity) and their chemical structures, 15 compounds were selected for further evaluation in secondary assays in order to select the best lead compound for subsequent medicinal chemistry. See FIG. 7

### Characterization of LDDN Small Molecule Hits in Secondary Assays

High-throughput screening of the LDDN compound library identified numerous small molecule hits capable of reducing the fluorescence signal from the cell-based BACE1 assay. While each compound carries the potential of being developed into a molecular probe or even a therapeutic agent, medicinal chemistry and structure-activity relationship studies require intensive labor and time to perform. To prioritize these small molecules, they were characterized in a series of secondary assays designed to confirm their activity and measure their potency. These initial experiments were performed using SY5Y-BACEGFP-SEAPAPPwt stable cells, and include 12-point dose-response curve determination, use of an in vitro BACE1 assay to identify potential direct BACE1 inhibitors, and an Aβ ELISA to verify that the compound hits target the amyloid cascade.

12-Point Dose-Response Curve Generation Identifies Numerous Potent inhibitors of sAPPβ

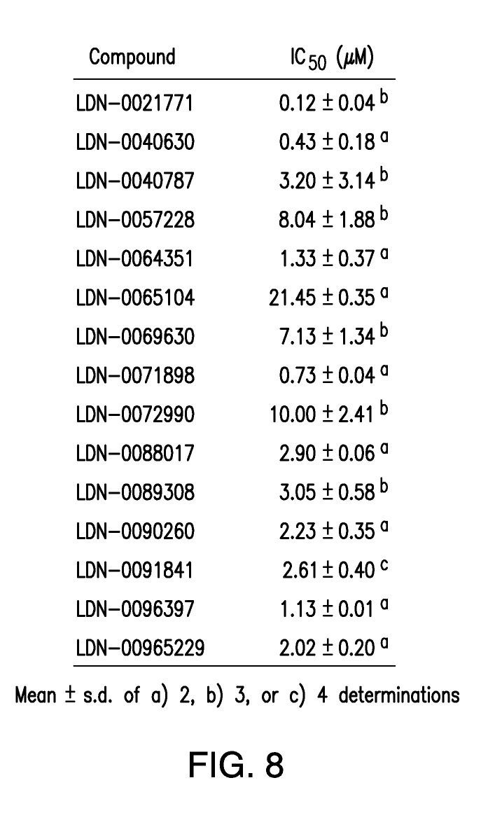

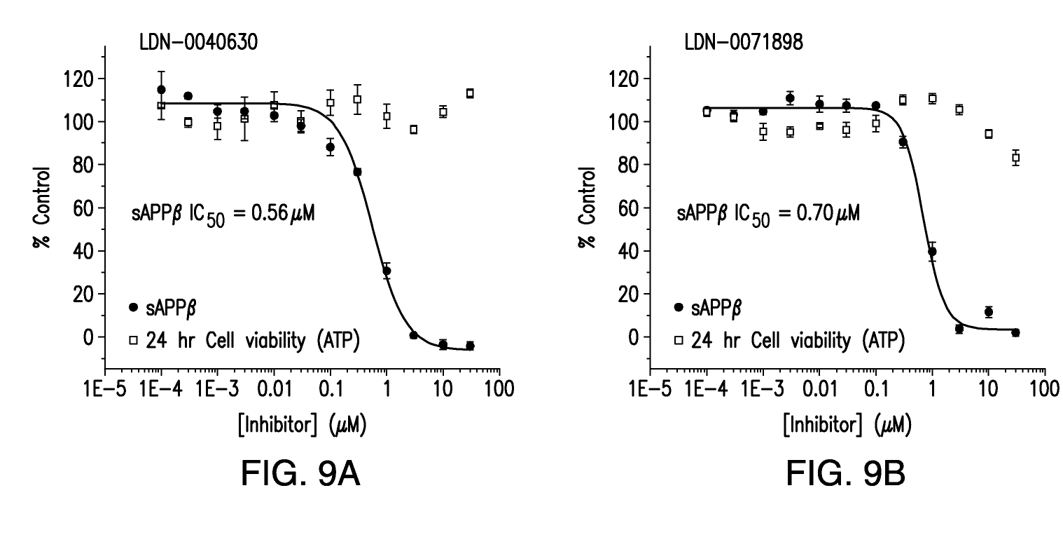

The 15 LDDN compounds selected based on their 3-point dose-response profile were tested at 12 concentrations (ranging from 0.1 nM to 30 μM) in the cell-based BACE1 assay. The assay was conducted in 96-well format to maximize the Z′ factor. Compounds were characterized in duplicate cell plates, each containing 12 doses of compound in triplicate. Cell viability was measured at 6 and 24 hours using Promega's Cell Titer-Glo kit according to the manufacturer's protocol. The dose-response curves for sAPPβ reduction were plotted using Origin software and fitted using a logistic model for IC₅₀ determination. These data are summarized in FIG. 8, and example curves are shown in FIG. 9.

IC₅₀ determination revealed 3 small molecules with sub-micromolar potencies and many more with potencies between 1 and 10 μM. The majority of these compounds are consistent with the efficacy, cytotoxicity, and chemical structure profiles suitable for medicinal chemistry and further studies.

In vitro BACE1 Assay Identifies Four Potential Direct BACE1 Inhibitors

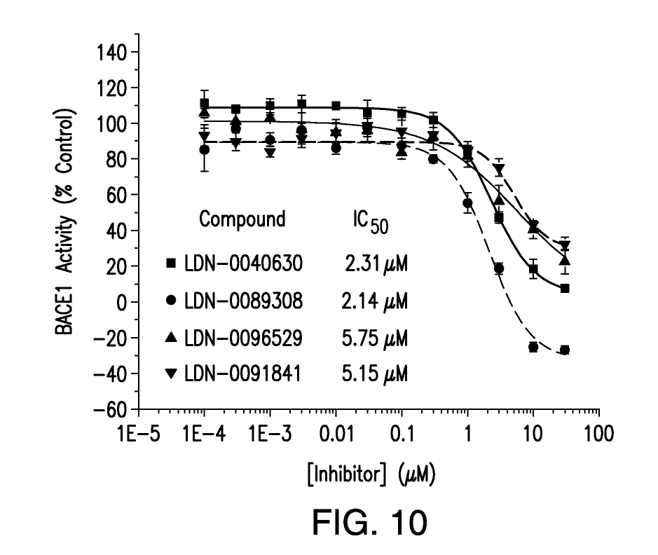

Because the cell-based BACE1 assay has the potential to uncover direct as well as indirect inhibitors of β-secretase, a commercial BACE1 enzymatic assay was employed to classify the small molecule hits. The BACE1 FRET Assay kit was purchased from Invitrogen and used according to the manufacturer's protocol. This fluorescence resonance energy transfer (FRET)-based assay measures the cleavage by purified recombinant β-secretase of a peptide substrate corresponding to the BACE1 cleavage site of Swedish mutant APP. The 15 LDDN hits were first tested at 3 concentrations (0.1, 1, and 10 μM) to determine if there is a dose-dependent inhibition of β-secretase. Four compounds, LDN-0040630, LDN-0089308, LDN-0096529, and LDN-0091841, exhibited a dose-dependent effect, and were re-characterized in the same enzymatic assay at 12 doses (FIG. 10).

Comparison of the IC₅₀ values obtained from the FRET-based BACE1 assay with those obtained from the cell-based BACE1 assay revealed some abnormalities. Because cell-based systems require the compound to pass through cellular membranes, compound potency in cell-based systems is usually orders of magnitude less than its potency in a direct enzymatic assay. Most of these four compounds have similar potencies in cell-based versus enzymatic assays (LDN-0089308—3.05 vs. 2.14 μM; LDN-0096529—2.02 vs. 5.75 μM; and LDN-0091841—2.61 vs. 5.15 μM). LDN-0040630, in particular, has a cell-based IC₅₀ (0.43 μM) an order of magnitude lower than its enzymatic IC₅₀ (2.31 μM).

Because the enzymatic assay is performed with compound in the reaction mixture (in contrast to the cell-based BACE1 assay, where the compound is washed away after antibody-mediated specific capture of sAPPβ), it is conceivable that the compound itself may interfere with the fluorescent readout of the enzymatic BACE1 assay. To explore this possibility, BACE1 substrate standard (cleaved peptide, supplied in the assay kit) was incubated with 12 concentrations of LDN-0040630, LDN-0089308, LDN-0096529, and LDN-0091841 in the absence of BACE1 enzyme. Under these conditions, all four compounds resulted in a maximal 25% inhibition of the fluorescence signal at the highest concentration used, which suggests that the compounds may interfere partially with the fluorescent readout of the assay, but do not account entirely for the fluorescence inhibition FIG. 10.

### 5 LDDN Compounds Reduce Aβ₄₀

Generation of β-amyloid is the central event in the amyloid cascade hypothesis, and the accumulation of Aβ is believed to lead to synaptic dysfunction and neurotoxicity. In conducting a cell-based high-throughput screen that monitors extracellular sAPPβ, it is possible to identify compounds that affect the degradation or the secretion of sAPPβ, and therefore do not target β-amyloidogenesis itself

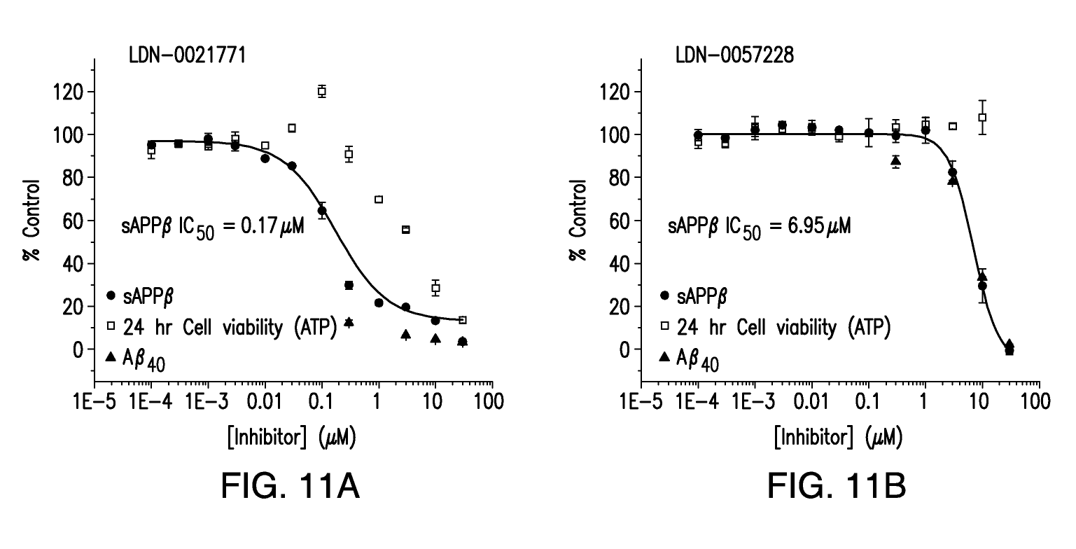

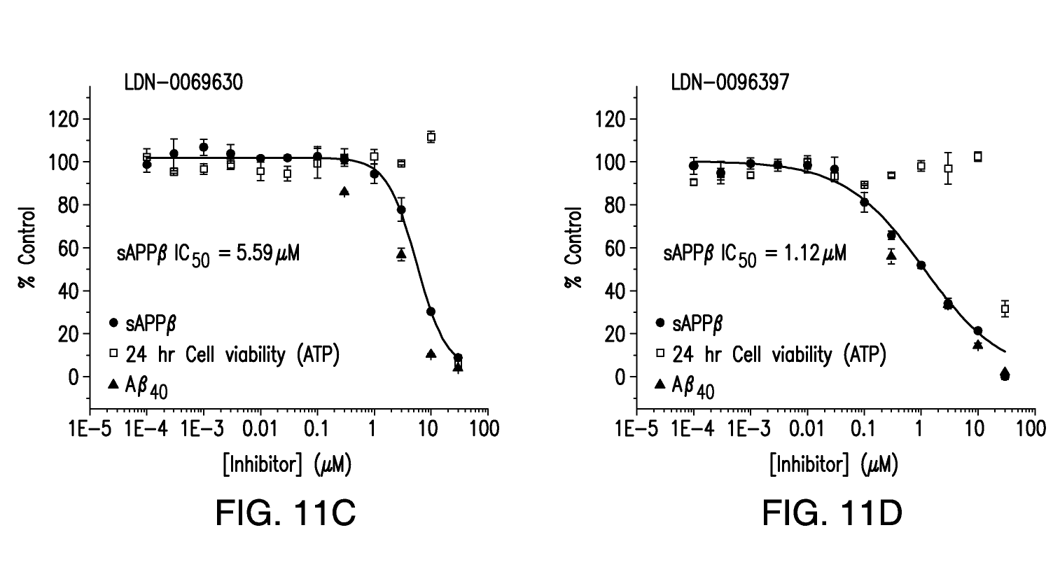

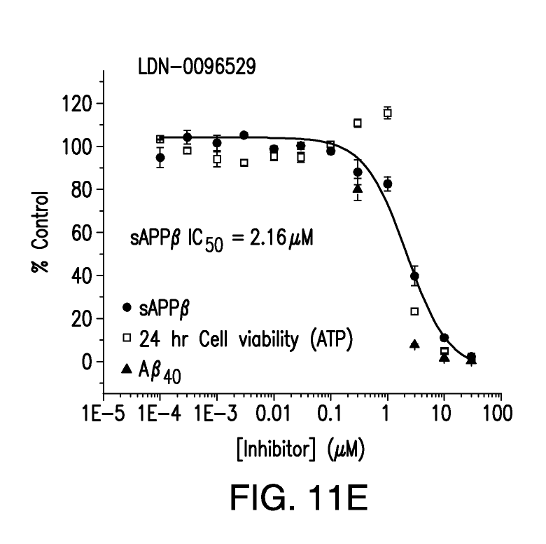

The 15 LDDN compounds were tested in SY5Y-BACEGFP-SEAPAPPwt cells for their ability to reduce Aβ₄₀ using a commercial Aβ₄₀ ELISA kit (BioSource). SY5Y-BACEGFP-SEAPAPPwt cells were grown to 100% confluence and incubated with 4 concentrations of each compound (30, 10, 3, 0.3 μM). Cell culture media was collected after 6 hours of compound incubation and diluted 3:10 in sample diluent supplied in the Aβ₄₀ ELISA kit. Aβ ELISA was performed according to the manufacturer's protocol. Of 15 LDDN compounds, 5 (LDN-0021771, LDN-0057228, LDN-0069630, LDN-0096397, and LDN-0096529) caused a dose-dependent decrease in Aβ₄₀. These data are shown in FIG. 11 together with their 12-point dose-response sAPPβ and 24-hour cell viability curves, demonstrating similar potencies for all 5 compounds in both the Aβ₄₀ ELISA and the cell-based BACE1 assay. LDN-0057228 and LDN-0069630 are small molecules with ample chemical space for modification, and are thus considered good candidates for medicinal chemistry.

### Selection of LDN-0057228 for SAR Studies

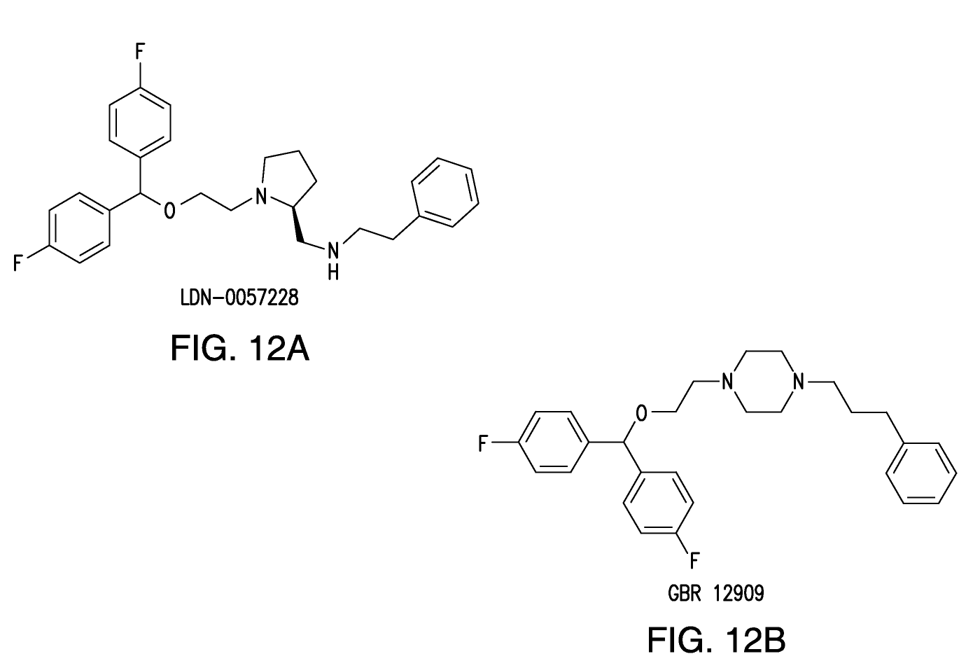

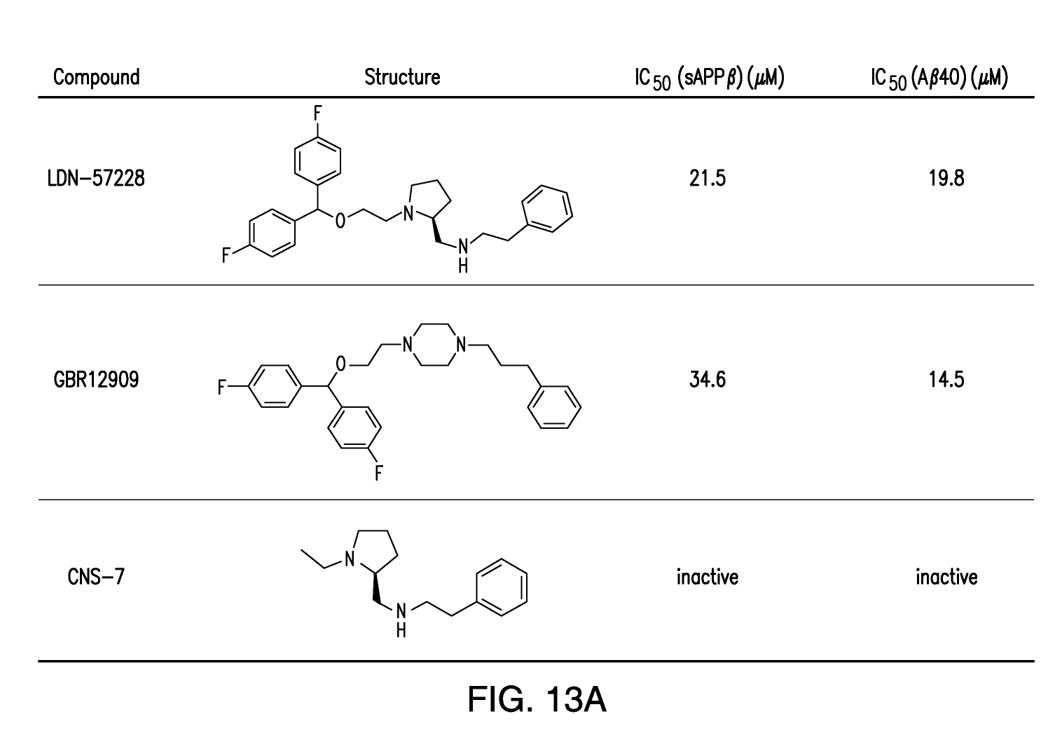

LDN-0057228 (FIG. 12A) is a piperazine ring altered analog of GBR compounds (aryl 1,4-dialkyl piperazines), which have been studied extensively as selective dopamine transporter (DAT) inhibitors and cocaine antagonists (Singh, 2000, Chem Rev., 100, 925-1024). GBR 12909 (FIG. 12B), a structural analog, exhibits potent inhibitory activity on DAT (IC₅₀=4.3 nM in vitro), and was shown to selectively block cocaine self-administration in rhesus monkeys via intravenous injection (Glowa et al., 1995, Exp Clin Psychopharm, 3, 219-239). The ability of GBR 12909 to traverse the blood-brain barrier, together with a potential known cellular target (DAT), makes LDN-0057228 the best candidate for medicinal chemistry and structure-activity relationship studies.

### Example 2

### Structure-Activity Relationship Studies and Characterization in Physiological Systems

Certain compounds identified in the screens of Example 1 were selected for medicinal chemistry. For example, 27 structural analogs of LDN-0057228 were synthesized. Subsequent characterization in SY5Y-BACEGFP-SEAPAPPwt cells identified CNS-2 as a potent analog. LDN-0057228 and CNS-2 were further characterized in a battery of more physiological assays for their ability to reduce Aβ₄₀ and sAPPβ. While LDN-0057228 and CNS-2 demonstrated activity in all systems tested, these studies strongly suggest that LDN-0057228 and CNS-2 are potent inhibitors of BACE1-mediated APP processing, and provides impetus for continued SAR and animal studies.

### SAR Studies of LDN-0057228

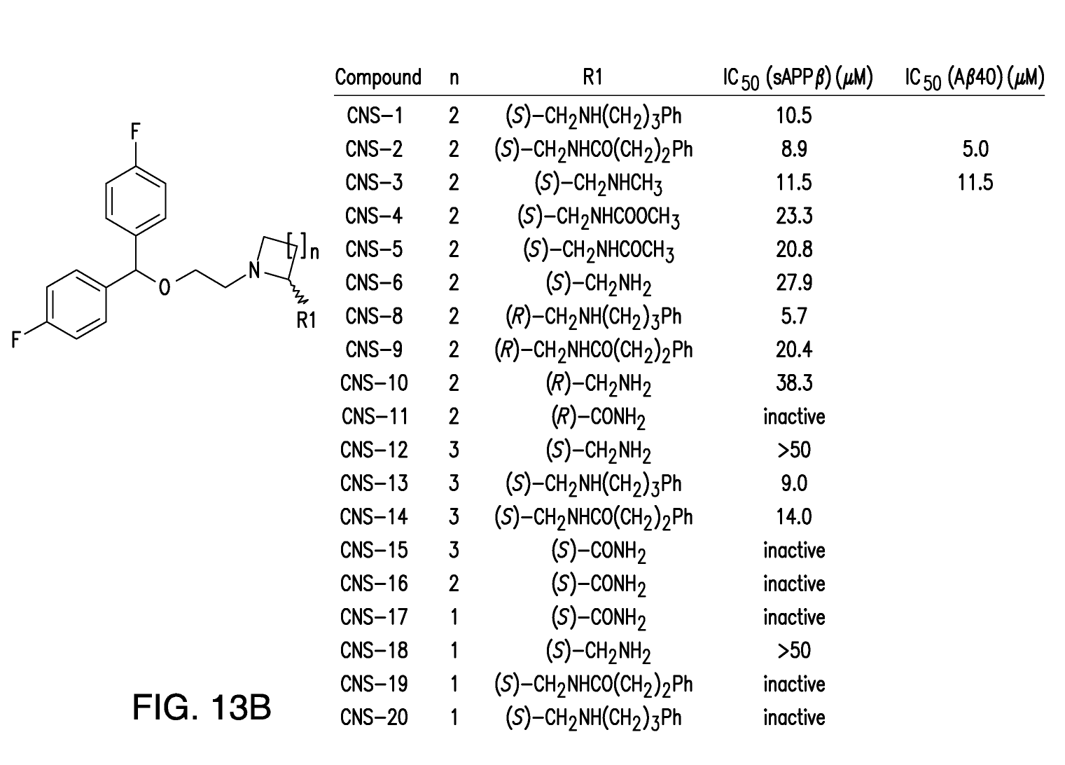

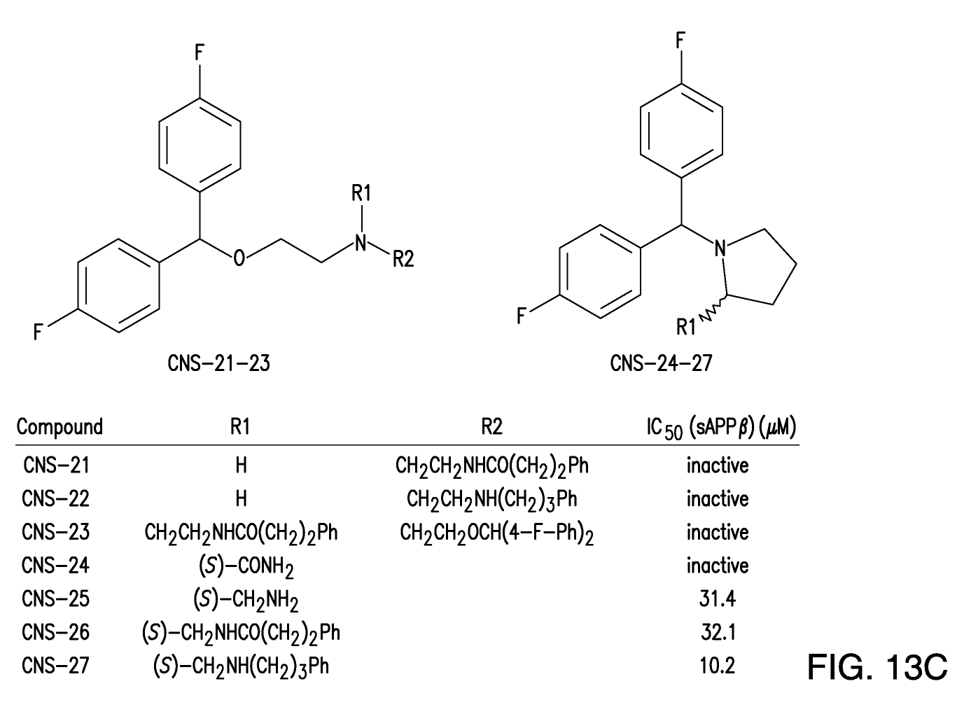

27 structural analogs of LDN-0057228 were synthesized and tested using the cell-based BACE1 assay in SY5Y-BACEGFP-SEAPAPPwt cells (FIG. 13). 4 analogs, in addition to the parent compound, were also assessed for Aβ₄₀ lowering activity using a commercial Aβ kit (BioSource). Because LDN-0057228 resembles CNS monoamine transporter inhibitors, this analog series was given the designation “CNS.” LDN-0057228 gave an unexpectedly low potency (IC₅₀˜20 μM for both sAPPβ and Aβ₄₀ reduction) compared to the ˜7 μM potency obtained using the same compound in FIG. 8.

GBR 12909, the potent dopamine transporter inhibitor, exhibited IC₅₀'s of 34.6 μM and 14.5 μM for sAPPβ and Aβ lowering, respectively, suggesting that DAT may be a cellular target of LDN-0057228 and its structural analogs (FIG. 13A). Analog CNS-7 was synthesized without the 4,4′-difluorobenzhydrol group, demonstrating that this portion of the molecule is critical for its activity (FIG. 13A). CNS analogs 1-6 and 8-20 were designed to vary the length and composition of the R1 group, as well as the size of the nitrogen-containing ring (FIG. 13B). Length and bulkiness of the R1 group seem to favor compound activity (e.g. CNS-1-5, 8-9 vs. CNS-6, 10, and 11). Stereochemistry of the R1 group seems to affect activity (e.g. CNS-1 vs. CNS-8, and CNS-2 vs. CNS-9). However, based on the available data it is unclear which is the favored stereochemistry. Expansion of the 5-membered ring to a 6-membered ring does not affect the activity. However, analogs with a 4-membered ring (CNS-17-20) were virtually all inactive.

Acyclic analogs (CNS-21-23) were all inactive, suggesting that the ring constrains the R groups in a conformation that is critical for compound activity (FIG. 13C). Finally, CNS analogs 24-27 moved the substituted pyrrolidine group closer to the 4,4′-difluorodiphenylmethane moiety, resulting in similar activity to CNS analogs 1-6 and 8-20 (FIG. 13C). This suggests that these two components are critical to the activity of the compound.

Although synthesis of analogs for LDN-0057228 failed to significantly improve the potency of the parent compound, valuable information was gained regarding the structure-activity relationship of this compound. CNS-2 was identified as one of the most potent analogs for sAPPβ reduction and exhibited the best potency for Aβ₄₀ reduction.

### Evaluation of LDN-0057228 and CNS-2 in More Physiological Systems

The use of the well-characterized tumor cell line SY5Y for primary screening and initial characterization, though convenient, has potential limitations. Perhaps most importantly, the transformed nature of these cells may give rise to phenotypes not observed in native neurons. The previously described hits were therefore further analyzed in more physiologically relevant systems, e.g. primary neurons and Alzheimer's model mice.

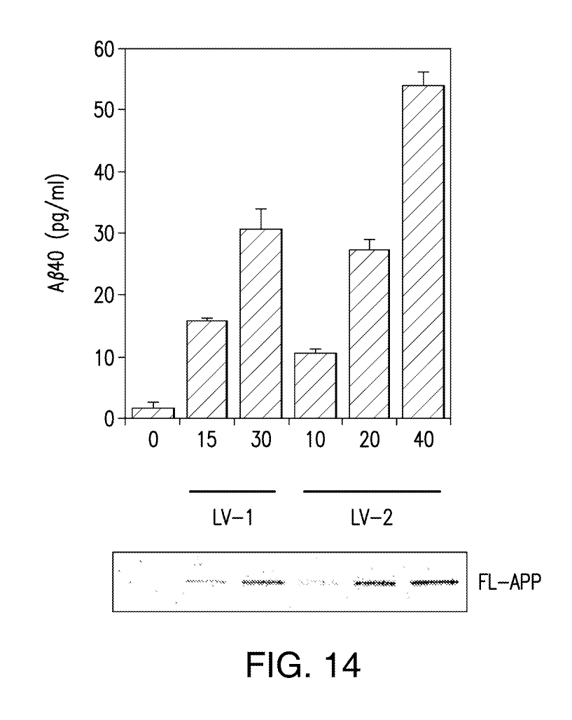

Two complementary neuronal cell systems were employed to test the effects of candidate compounds on various aspects of APP processing: cultured mouse cortical neurons (postnatal day 0) infected with recombinant lentivirus carrying human APPsw (Lenti-APPsw; FIG. 14); and cultured cortical neurons prepared from Tg2576 mice (carrying human APPsw transgene under the control of the PrP promoter). Lenti-APPsw infected cortical neurons can be prepared in a relatively large scale, but suffer from variability in lentiviral infection efficiency, resulting in variable levels of APP expression. In contrast, cortical neurons derived from Tg2576 provide constant APP expression. However, neuronal yield is generally much lower, since only half the pups will contain the transgene when heterozygote crossbreeds were conducted. Therefore, Initial characterization experiments were performed using cortical neurons infected with Lenti-APPsw. Compounds exhibiting inhibitory activity on sAPPβ and/or Aβ would then be re-evaluated in Tg2576 mice-derived neurons.

It is conceivable that compound activity in cultured cells or neurons may not correlate with that in the intact brain. Thus, a battery of ex vivo and in vivo assays would also be used to characterize the most promising hit(s). Ex vivo systems, such as organotypic brain slices, offer a good alternative to in vivo assays, and can also be used to test a large number of compounds. Furthermore, at least with regard to the Aβ release phenotype, brain slice data have been shown to correlate well with the results obtained in vivo (reviewed in Noraberg et al., 2005, Curr Drug Targets CNS Neurol Disord, 4(4):435-52). However, because brain slices have a definite thickness, and are cultured above a membrane filter, it is possible that the compound may not penetrate sufficiently to cause a significant effect. Despite this drawback, organotypic brain slices offer a good ex vivo system, and brain slices from p7 Tg2576 pups were used for compound characterization.

For in vivo studies, interstitial fluid (ISF) compound administration and Aβ measurement, as well as intraperitoneal (IP) compound injection, both using Tg2576 mice were used. Positioning of a guide cannula to the mouse hippocampus allows for insertion of a microdialysis apparatus, which can be used to infuse compounds at multiple concentrations sequentially in the awake mouse. Aβ measurements can be performed using the same apparatus, yielding rapid dose-response determinations (Cirrito et al., 2003, J Neuroscience, 23(26):8844-8853). This method allows for rapid assessment of compound effect on Aβ on a dynamic time scale. More conventionally, compounds can also be administered via IP injection. For these experiments, we obtained 12-month old Tg2576 mice from the Duff lab.

LDN-0057228 and CNS-2 Reduce sAPPβ and Aβ40 in Lenti-APPsw Infected Primary Cortical Neurons

Lenti-APPsw infected primary cortical neurons were selected for the initial round of physiological experiments due to the relatively large batches of wild-type primary cortical neurons that were routinely harvested in the lab, the ease of lentiviral packaging. Primary cortical neurons were harvested from wild-type P0 pups using established protocols. The majority of cells from the resulting culture exhibit neuronal morphology on light microscopy and express neuronal β-tubulin-which can be visualized by immunocytochemistry using the TUJ1 antibody (Covance).

The Lenti-APPsw vector was co-transfected into HEK293 T cells with ViraPower packaging mix (Invitrogen) to generate the lentivirus. Lentiviral-mediated transduction of APPsw in primary neurons was performed by adding neuron primary culture media containing the lentiviral particles to wild-type DIV-14 primary neurons (FIG. 14). After 24 hours, neurons were incubated with media for 72 hours prior to collecting for Aβ₄₀ measurement. Two batches of virus (LV-1 and LV-2) were tested, showing that there is batch-to-batch variation in the viral titer.

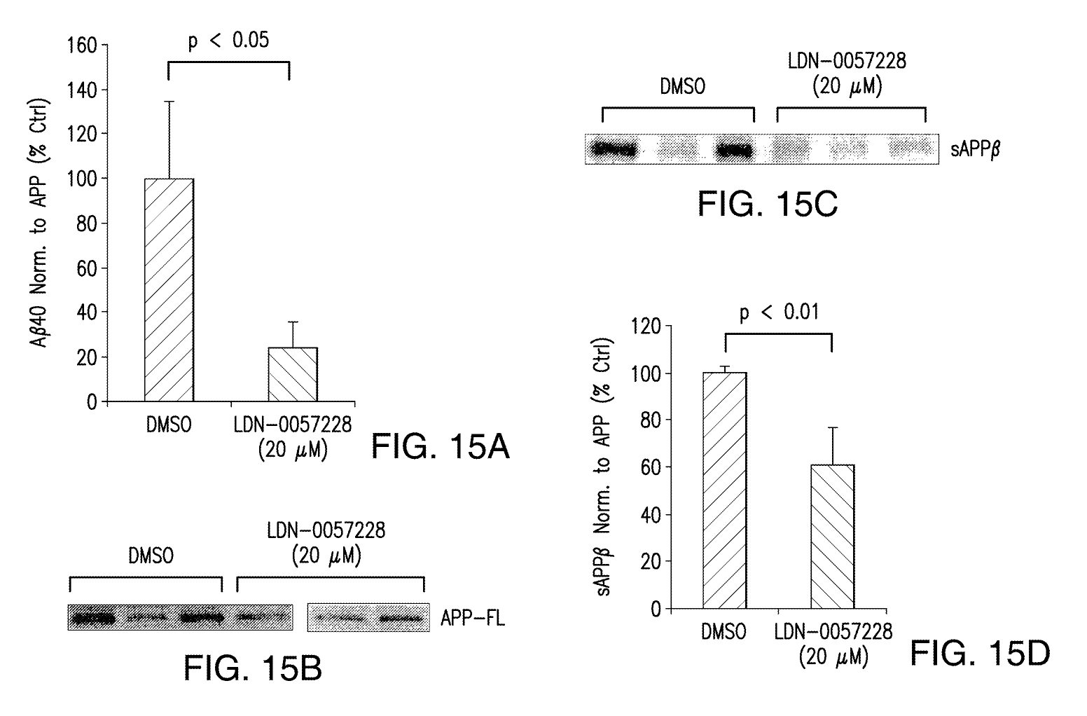

Using this experimental paradigm, LDN-0057228 was tested at 20 μM concentration to confirm the sAPPβ- and Aβ-lowering activity of the compound (FIG. 15). The protocol was modified slightly to allow for 24-hour compound treatment. DIV-14 wild-type primary neurons cultured on 6-well plates were incubated for 24 hours with primary culture media containing lentiviral particles. After infection, fresh media was mixed in a 1:1 ratio with conditioned media collected prior to lentiviral infection, and applied to the cells for 48 hours to allow for APP expression. At the end of this “pre-drug” incubation, media containing compound was applied for 24-hour treatment. Control experiments using DMSO alone showed that media collected after the 24-hour treatment period contained at least two times higher Aβ₄₀ than that collected just after the 48-hour pre-drug period (data not shown).

LDN-0057228 caused a 75% reduction in Aβ₄₀ levels (p<0.05) in primary neurons (FIG. 15A). The Aβ results were normalized to total APP as visualized on Western blot in FIG. 15B. It is evident from FIG. 15B that there is significant variability in APP expression from well to well. This variability is believed not to be due to a compound-mediated effect on APP expression, as DMSO-treated wells were also affected. sAPPβ was immunoprecipitated from the media with sβsw antibody and visualized on Western blot using LN27 antibody (FIG. 15C). With the exception of one outlier in the DMSO group (which also had significantly lower APP expression), there was a clear reduction of sAPPβ in the LDN-0057228-treated wells. The sAPPβ bands were quantified using ImageJ software and normalized to APP, showing a 40% reduction in sAPPβ (p<0.01) for the treated neurons (FIG. 15D).

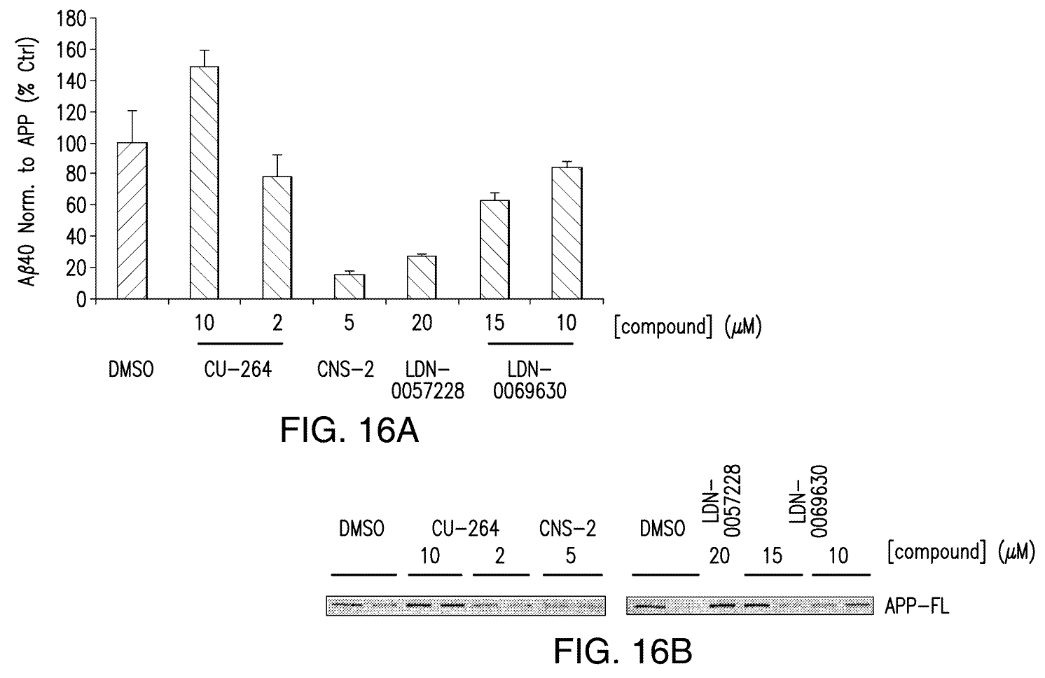

CNS-2 and LDN-0069630 were also characterized using the same experimental paradigm (FIG. 16). CNS-2, the potent structural analog of LDN-0057228, reduced Aβ₄₀ by >80% at 5 μM. LDN-0057228 was repeated in one well in this experiment, and reduced Aβ by 75% at 20 μM, as before. LDN-0069630, the other LDDN small molecule with Aβ-lowering activity in SY5Y-BACEGFP-SEAPAPPwt cells, caused a 15% and 40% reduction in Aβ₄₀ at 10 and 15 μM, respectively. However, due to the large variability in APP expression in the DMSO-treated wells, these reductions did not reach statistical significance

LDN-0057228 and CNS-2 Reduce sAPPβ and Aβ₄₀ in Tg2576 Primary Cortical Neurons

Culturing primary neurons from Tg2576 pups offers the advantage of equal APP expression, but suffers the drawback of lower yield since only half the pups contains the transgene. Furthermore, because neurons from each pup has to be plated separately, plating density and neuronal survivability may vary from mouse to mouse. Thus, total protein was used to normalize the data.

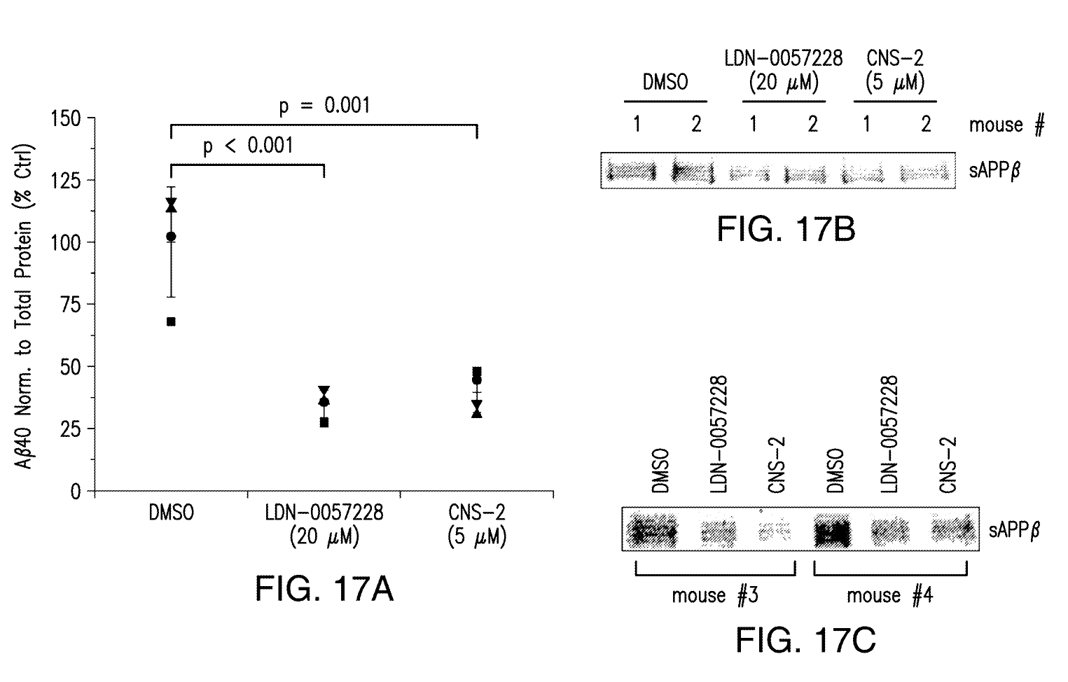

CNS-2 and LDN-0057228 were evaluated in the Tg2576 pup system (FIG. 17). Primary cortical neurons were harvested from P0 Tg2576 pups. Each pup yielded 3 wells on a 12-well plate at a plating density of 0.8×10⁵ cells per well. After genotyping, neurons from each transgenic pup (APPsw +/−) were treated with DMSO, LDN-0057228 (20 μM), or CNS-2 (5 μM) at DIV-14 for 24 hours. Data from two independent experiments were pooled, representing primary neurons from 4 transgenic pups. LDN-0057228 (20 μM) reduced Aβ₄₀ by 65% (p<0.001) and CNS-2 (5 μM) reduced Aβ₄₀ by 60% (p=0.001) (FIG. 17A). Both compounds also reduced sAPPβ as visualized by IP-Western (FIG. 17B).

Data from the two complementary neuronal cell systems indicate that LDN-0057228 as well as its structural analog, CNS-2, affect BACE1-mediated cleavage of APP.

### CNS-2 Reduces Brain Total Aβ₄₀ in Tg2576 Mice

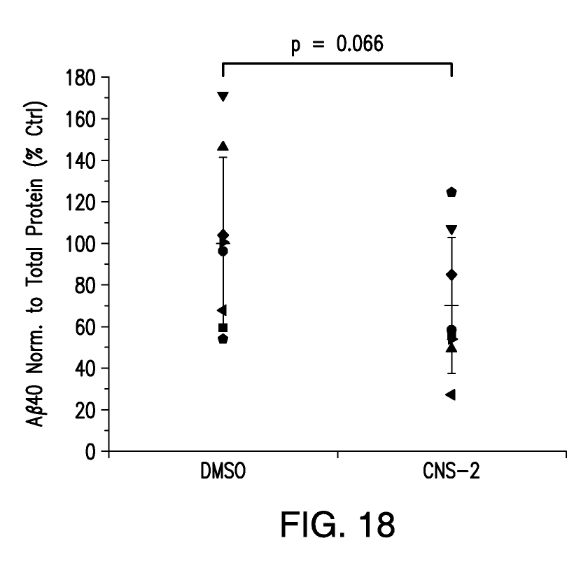

A total of 16 mice were treated with either DMSO (n=8) or 3 mg/kg CNS-2 (n=8). The dosage was selected arbitrarily based on compound solubility in 0.9% normal saline. CNS-2 was dissolved in 0.9% normal saline solution with 1.9% final DMSO concentration. 12- to 13-month old Tg2576 mice were treated with DMSO or CNS-2 via intraperitoneal injection for 9 days (1 injection per day) at an injection volume of 20 μl per gram of weight. Mouse weight was monitored daily, and the total injection volume adjusted accordingly over the course of the 9-day treatment. Neither the treated nor control groups exhibited any significant changes in weight or any overt signs of toxicity. Mice were sacrificed on day 9, 5 hours after the final injection. One hemibrain from each mouse was homogenized and processed for formic acid extraction of plaque Aβ. Total Aβ₄₀ was determined by Aβ ELISA kit and normalized to total protein (FIG. 18).

CNS-2 reduced Aβ₄₀ by 30%, although the p value was greater than 0.05. As preliminary data, these results are encouraging because we had no prior information regarding the pharmacokinetics of CNS-2. The extent of drug metabolism and the compound's ability to penetrate the blood-brain barrier were unknown.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, GenBank Accession Numbers, and protocols are cited throughout this application, the disclosures of which are incorporated herein by reference in their entireties for all purposes.

1. A pharmaceutical composition comprising a therapeutically effective amount of a compound of Formula I: 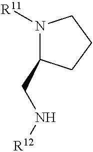
wherein R¹¹ and R¹² are independently selected for each occurrence from the group consisting of substituted or unsubstituted alkyl, cycloalkyl, aryl, heteroaryl and alkenyl;
and salts, esters and prodrugs thereof, and a pharmaceutical carrier.

2. The pharmaceutical composition of claim 1, wherein R¹¹ is independently selected for each occurrence from the group consisting of ethyl and:

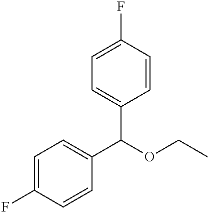

3. The pharmaceutical composition of claim 1, wherein R¹² is independently selected for each occurrence from the group consisting of hydrogen, methyl, COCH₃ and:

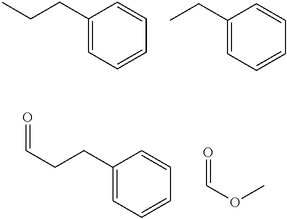

4. A pharmaceutical composition comprising a therapeutically effective amount of a compound of Formula II: 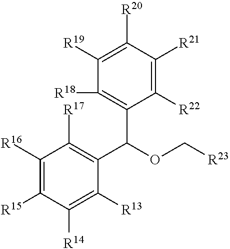
wherein R²³ is selected from the group consisting of substituted or unsubstituted alkyl, cycloalkyl, aryl, heteroaryl and alkenyl, and wherein R¹³-R²² are independently selected for each occurrence from the group consisting of hydrogen, halogen, alkyl, aryl, CN, alkoxy, aryloxy, NO₂, alkylthio, and arylthio;
and salts, esters and prodrugs thereof, and a pharmaceutical carrier.

5. The pharmaceutical composition of claim 4, wherein R¹³-R¹⁴, R¹⁶-R¹⁹, and R²¹-R²² are hydrogen and R¹⁵ and R²⁰ independently selected for each occurrence from the group consisting of hydrogen, F, Cl, and Br, and R²³ is a substituted alkyl.

6. A pharmaceutical composition comprising a therapeutically effective amount of a compound of Formula III: 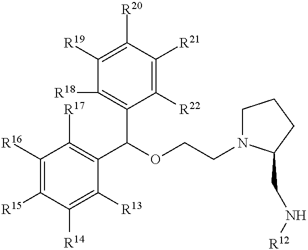
wherein R¹² is selected from the group consisting of substituted or unsubstituted alkyl, cycloalkyl, aryl, heteroaryl and alkenyl; and
wherein R¹³-R²² are independently selected for each occurrence from the group consisting of hydrogen, halogen, alkyl, aryl, CN, alkoxy, aryloxy, NO₂, alkylthio, and arylthio;
and salts, esters and prodrugs thereof, and a pharmaceutical carrier.

7. The pharmaceutical composition of claim 6, wherein the compound is:

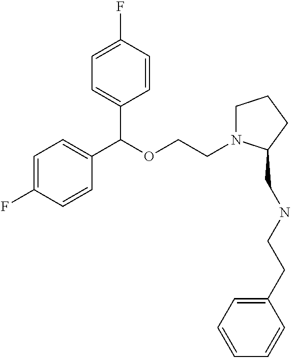

8. The pharmaceutical composition of claim 6, wherein R¹³-R²² are independently selected for each occurrence from the group consisting of hydrogen and halogen.

9. The pharmaceutical composition of claim 6, wherein R¹³-R¹⁴, R¹⁶-R¹⁹, and R²¹-R²² are hydrogen; and
wherein R¹⁵ and R²⁰ are independently selected for each occurrence from Cu the group consisting of hydrogen and halogen, (ii) the group consisting of hydrogen, F, Cl, and Br, (iii) the group consisting of F, Cl, and Br, or (iv) F.

10. The pharmaceutical composition of claim 6, wherein R¹³-R¹⁴, R¹⁶-R¹⁹, and R²¹-R²² are hydrogen;
wherein R¹⁵ and R²⁰ are F; and
wherein R¹² is (R)—CH₂NH(CH₂)₃Ph.

11. The pharmaceutical composition of claim 6, wherein R¹³-R¹⁴, R¹⁶-R¹⁹, and R²¹-R²² are hydrogen;
wherein R¹⁵ and R²⁰ are F; and
wherein R¹² is (S)—CH₂NHCO(CH₂)₂Ph.

12. The pharmaceutical composition of claim 6, wherein R¹³-R¹⁴, R¹⁶-R¹⁹, and R²¹-R²² are hydrogen;
wherein R¹⁵ and R²⁰ are F; and

13. A pharmaceutical composition comprising a therapeutically effective amount of a compound of Formula IV: 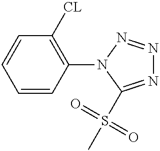
and salts, esters and prodrugs thereof, and a pharmaceutical carrier.

14. A pharmaceutical composition comprising a therapeutically effective amount of a compound of Formula V: 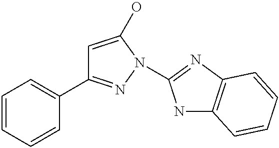
and salts, esters and prodrugs thereof, and a pharmaceutical carrier.

15. A pharmaceutical composition comprising a therapeutically effective amount of a compound of Formula VI: 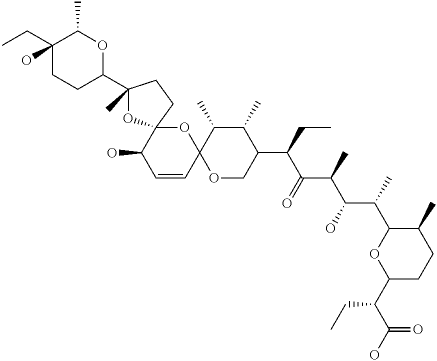
and salts, esters and prodrugs thereof, and a pharmaceutical carrier.

16. A pharmaceutical composition comprising a therapeutically effective amount of a compound of Formula VII: 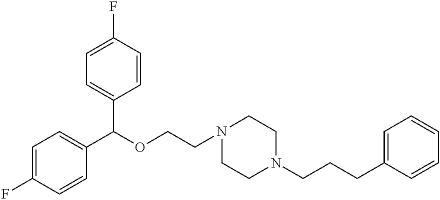
and salts, esters and prodrugs thereof, and a pharmaceutical carrier.

17. A method for inhibiting the activity of a β-site APP cleavage enzyme 1 (BACE1) in a cell which comprises contacting the cell with a compound of Formula I, II, III, IV, V, VI, or VII in an amount effective to inhibit β-site APP cleavage enzyme 1 activity.

18. The method of claim 17, wherein the inhibition of β-site APP cleavage enzyme 1 activity reduces the metabolism of an amyloid precursor protein (APP).

19. The method of claim 17, wherein the cell is a mammalian cell.

20. The method of claim 17, wherein the cell is contacted in vitro.

21. A method for treating Alzheimer's disease in an individual, which method comprises administering to the individual an effective amount of a compound of Formula I, II, III, IV, V, VI, or VII.
